(19) United States
(12) Patent Application Publication
(10) Pub. No.: US 2023/0405146 A1
(43) Pub. Date: Dec. 21, 2023

(54) Antibody-conjugated nanoparticles

(71) Applicants: HARBOUR ANTIBODIES BV, Rotterdam (NL); ERASMUS UNIVERSITY MEDICAL CENTER ROTTERDAM, Rotterdam (NL); LEIDEN UNIVERSITY MEDICAL CENTER, Leiden (NL)

(72) Inventors: Frank G. GROSVELD, Rotterdam (NL); Christina EICH, Leiden (NL); Luis CRUZ, Leiden (NL)

(21) Appl. No.: 18/251,988

(22) PCT Filed: Nov. 5, 2021
(86) PCT No.: PCT/EP2021/080755

§ 371 Date: May 5, 2023

(30) Foreign Application Priority Data
Nov. 6, 2020 (GB) 2017555.0

(51) Int. Cl. A61K 47/69; C12N 9/22; C07K 16/28; C12N 15/10; A61P 7/00

(52) U.S. Cl. CPC A61K 47/6929; C12N 9/22; C07K 16/2896; C07K 16/2878; C12N 15/102; A61P 7/00

(57) ABSTRACT

The invention relates to antibody conjugates comprising an antibody and a nanoparticle conjugated to the antibody, wherein the nanoparticle comprises a payload, such as a gene editing payload. In some embodiments, the antibody-conjugated nanoparticles provide a means for treating a disease. In some embodiments, the antibody-conjugated nanoparticles are used to correct genetic defects in specific cell populations.

### CROSS REFERENCE TO RELATED APPLICATIONS

The present application is a United States National Phase under 35 U.S.C. § 371 of International Application No. PCT/EP2021/080755, filed Nov. 5, 2021, which claims priority to United Kingdom Patent Application No. 2017555.0, filed on Nov. 6, 2020, the entire contents of each which are incorporated by reference herein.

### BACKGROUND

Haematological malignancies, and non-oncological inherited disorders, such as sickle cell disease (SCD) and β-thalassemia, are caused by mutations that affect the proper function of the hematopoietic system. With the increase in life expectancy, the number of elderly people diagnosed with a haematological condition has increased substantially. Treatment and prevention of haematological disorders remains challenging, given the dynamic nature of the hematopoietic system and the genetic heterogeneity among disorders and patients. Hematopoietic stem cells (HSCs) offer great opportunities to develop novel treatments for numerous malignant and non-malignant diseases. Their ability to self-renew and differentiate into all blood lineages has already been utilized for stem cell therapies. Novel treatments, such as gene-editing, aim to manipulate autologous HSCs to reverse disease symptoms. In other situations, it might be necessary to eliminate diseased/exhausted HSCs (e.g. in the process of aging), block receptors on hematopoietic stem and progenitor cells (HSPCs) (e.g. in acute myeloid leukaemia), or in HSC transplantation the recipient HSCs need to be specifically eliminated by cytotoxic drugs, while keeping non-hematopoietic cells unaffected (e.g. during bone-marrow transplantation).

In the case of gene-editing, manipulation of allogeneic or autologous HSCs is performed ex vivo and the edited HSCs are reinfused into the patient. However, this poses several disadvantages: (1) the risk of cell differentiation and loss of homing/engraftment potential of the purified target cells; (2) a lack of precise markers that permit the isolation of sufficient long-term repopulating HSCs; (3) the pre-transplant conditioning used to “make space” for engraftment of the isolated and reinfused HSCs has severe acute toxicities in multiple organs, which is a restriction in the treatment of elderly patients; (4) the ex-vivo gene-editing of HSCs requires specialized health-care centres and only few patients have access, in particular patients in less developed parts of the world are excluded.

Efficient gene-editing in HSPCs has been achieved using electroporation and/or viral transduction to deliver CRISPR, but cellular toxicity is a drawback of currently used methods. Nanoparticle (NP)-based gene-editing strategies could further enhance the gene-editing potential of HSPCs and provide a delivery system for in vivo application.

However, a bottleneck in the development of HSC-targeted therapies is the lack of suitable and a specific HSC-delivery systems.

One of the limitations in the development of HSC-targeted therapies is the lack of unique HSC cell surface markers. For example, CD34 is commonly used to isolate HSCs from blood and bone marrow, but marks a heterogeneous cell population including immune and endothelial cells. Recently, the G-protein coupled receptor gpr56 was described as a novel marker expressed on HSCs, and all murine long-term repopulating HSCs were shown to express gpr56.

Targeting known HSC cell surface receptors in vivo may be greatly improved by utilizing high-affinity classical antibodies (H2L2 Abs) to enhance higher endocytic capacity in HSPCs. Newly developed human heavy-chain only antibodies or their VH-region alone, may also improve targeting towards HSCs by the possibility to engineer dual specificity Abs. Both types of antibodies have the added advantage of being human which avoids an immune reaction.

Many therapeutic compounds and gene-editing components are prone to degradation and possess poor membrane permeability potential. For in vivo delivery to HSCs, sophisticated non-toxic delivery systems would be needed. Nanoparticles (NPs) made from biodegradable and FDA-approved PLGA protect their payload from premature degradation and guide the payload into target cells. High payload delivery by PLGA-NPs improves the ratio of efficacy/toxicity; this is highly desirable for HSCs which are sensitive to the cytotoxicity of traditional gene targeting procedures.

### SUMMARY OF THE INVENTION

The invention provides an antibody conjugate comprising an antibody and a nanoparticle conjugated to the antibody, wherein the nanoparticle comprises a gene editing payload.

The invention further provides an ex vivo method for gene editing comprising administering an antibody conjugate comprising a gene editing payload to a population of cells comprising cells that express a surface antigen which is specifically bound by the antibody.

The invention further provides a method of treating a disease comprising administering an antibody conjugate comprising a gene editing payload to a patient.

The invention further provides an antibody conjugate comprising an anti-CD34 antibody and a nanoparticle conjugated to the antibody, wherein the nanoparticle comprises a payload. Additionally, the invention provides an antibody conjugate comprising an anti-Gpr56 antibody and a nanoparticle conjugated to the antibody, wherein the nanoparticle comprises a payload.

Also provided herein is a method of making an antibody conjugate, comprising: modifying an antibody heavy chain coding sequence to introduce a cysteine residue at or near the C-terminal end of the heavy chain constant region; producing a modified antibody from the modified sequence, wherein the modified antibody comprises a cysteine residue at or near the C-terminal end of the heavy chain constant region, wherein said cysteine residue has a free thiol group that is not covalently bonded to another cysteine residue; obtaining a poly(lactic-co-glycolic acid)(PLGA)-nanoparticle comprising PEG; and conjugating the nanoparticle to the antibody via a site-specific maleimide linkage, wherein the cysteine residue at or near the C-terminal end of the heavy chain constant region is covalently bonded to one of the one or more PEG groups of the nanoparticle.

The invention further provides a method of treating or ameliorating the symptoms of a genetic disorder comprising administering to a patient a composition comprising an antibody conjugate, wherein said antibody conjugate comprises an antibody and a nanoparticle conjugated to the antibody, and said nanoparticle comprises a gene editing payload. Additionally, the invention provides an antibody conjugate comprising an antibody and a nanoparticle conjugated to the antibody for use in the treatment or amelioration of the symptoms of a genetic disorder, wherein the nanoparticle comprises a gene editing payload.

The invention provides a method of treating or ameliorating the symptoms of a disease comprising administering to a patient a composition comprising an antibody conjugate, wherein said antibody conjugate comprises an anti-Gpr56 antibody and a nanoparticle conjugated to the antibody, and said nanoparticle comprises a therapeutic payload. Additionally, the invention provides an antibody conjugate comprising an anti-Gpr56 antibody and a nanoparticle conjugated to the antibody for use in the treatment or amelioration of the symptoms of a disease, wherein the nanoparticle comprises a therapeutic payload.

The invention also provides a method of treating or ameliorating the symptoms of a disease comprising administering to a patient a composition comprising an antibody conjugate, wherein said antibody conjugate comprises an anti-CD34 antibody and a nanoparticle conjugated to the antibody, and said nanoparticle comprises a therapeutic payload. Additionally, the invention provides an antibody conjugate comprising an anti-CD34 antibody and a nanoparticle conjugated to the antibody for use in the treatment or amelioration of the symptoms of a disease, wherein the nanoparticle comprises a therapeutic payload.

The invention further provides an anti-CD34 antibody, comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 1; an HCDR2 comprising the amino acid sequence of SEQ ID NO: 2; and an HCDR3 comprising the amino acid sequence of SEQ ID NO: 3; and the VL comprises an LCDR1 comprising the amino acid sequence of SEQ ID NO: 4; an LCDR2 comprising the amino acid sequence of SEQ ID NO: 5; and an LCDR3 comprising the amino acid sequence of SEQ ID NO: 6.

The invention also provides an anti-Gpr56 antibody, comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 9; an HCDR2 comprising the amino acid sequence of SEQ ID NO: 10; and an HCDR3 comprising the amino acid sequence of SEQ ID NO: 11; and the VL comprises an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

### FIGURES

FIG. 1. Expression pattern of CD34 and gpr56 in cord and peripheral blood. (A) Flow cytometric dot plots showing gating and percentages of CD34+, Gpr56+ and CD34+gpr56+ cells in cord blood (left) and peripheral blood (PBMCs, right). Viable blood cells were gated based on FSC-A and SSC-A (no debris), followed by exclusion of 7-AAD+ cells (dead cells). Viable cells were further analysed for expression of CD34 (CD34-PE-Cy7, commercial antibody) and gpr56 (gpr56-PE, commercial antibody). (B) PBMC gating strategy based on CD34/gpr56 double labelling. CD34+gpr56−, CD34+gpr56+ and CD34-gpr56+ cells were sorted and plated into methylcellulose. (C) Hematopoietic potential correlates with gpr56 expression. Hematopoietic progenitor numbers in cord blood CD34+, gpr56+, CD34+gpr56−, CD34+gpr56+ and CD34− gpr56+ sorted cells. CFU-C (colony forming unit-culture) per 700 sorted cells is shown, with colony types designated by coloured bars: CFU-G=CFU-granulocyte, CFU-M=macrophage and CFU-GM=CFU-granulocyte, macrophage, CFU-GEMM=CFU-granulocyte, erythroid, macrophage, megakaryocyte, and BFU-E=burst forming unit erythroid.

FIG. 2. Generation of novel, fully human α-gpr56 and α-CD34 antibodies. (A) Schematic overview of the generation of fully human antibodies. Human gpr56 and CD34 extracellular domains were expressed in HEK 293 cells as his-tagged fusion proteins (not shown) and used for immunization of H2L2 transgenic mice (Harbour) followed by the generation of hybridomas and the cloning of the antigen binding antibodies. (B) Schematic representation of selected mAbs. Two clones with high affinities for human gpr56 (clone 3.8g8) and CD34 (L5F1) were selected to generate fully human α-gpr56 and α-CD34 antibodies. Of each clone, a standard antibody format and a format with two extra cysteines at the Fc region were generated. (C) Fully human α-gpr56 (clone 3.8g8) and α-CD34 (L5F1) antibodies (no extra Cysteine) were directly conjugated to Atto-647-NHS-ester, at a degree of labelling of 2 molecules dye/molecule antibody. Cord blood was labelled with α-CD34-PE-Cy7 and α-gpr56-PE commercial mAbs) antibodies, or α-gpr56-Atto647 (clone 3.8g8) and α-CD34-Atto647 (clone L5F1).

FIG. 3. Conjugation strategies of antibodies to PLGA-PEG-NPs. (A) Schematic representation of the fully human H2L2 antibody, incorporating two extra cysteines with free thiol groups at the Fc regions. (B) Schematic representation of the PLGA-PEG-NP. (Left) Conjugation using NHS-ester randomly cross-links the antibody to the PLGA-PEG (—NH2)-NP surface via any lysine present in the antibody sequence. (Right) Conjugation via maleimide-cysteine is specific for free cysteines at the Fc tail. All other cysteines present in the antibody sequence are engaged in disulfide-bridges with neighbouring cysteines. This site-specific conjugation strategy guarantees the proper orientation of all antibody variable regions (and antigen binding grooves) away from the NP-core. (C) Representative TEM images of CD34-PLGA-PEG-NPs. Scale bars indicated. (D) Representative confocal images of empty- and CD34-PLGA-PEG-NPs. Red=anti-human Fc-AF488. Scale bars indicated.

FIG. 4. α-gpr56-PLGA-PEG-NP and α-CD34-PLGA-PEG-NP specifically target HSPCs in blood. (A) Flow cytometric dot plots showing gating of CD34− and CD34+ cells in peripheral mononuclear blood cells (PBMCs). (B) A representative analysis of the viable CD34+ and viable CD34− population incubated with α-CD34-PLGA-PEG-NPs. PBMCs were incubated for 15 minutes with 20 μg/ml α-CD34(Cys)-PLGA-PEG-NP conjugated via NHS-ester and via maleimide-thiol, extensively washed and analysed by flow cytometry. Viable blood cells (7-AAD−) were gated as CD34+ and CD34− cells, and the subpopulations were further analysed for the expression of DiD; the fluorophore encapsulated inside of the PLGA-PEG-NPs. (C) Binding of NPs (conjugated to α-gpr56(Cys), α-CD34(Cys), α-MSLN (Cys) and α-Strep via NHS-ester or maleimide-thiol reaction) to CD34+ and CD34− blood cells was evaluated. The percentage of CD34+ and CD34− cells that had bound the fluorescent carriers was determined by flow cytometry. The data represents the mean±SEM of 4-7 independent experiments, including at least 3 different donors. Statistical significance was calculated using a 2-way ANOVA and Bonferroni's multiple comparison test. agpr56- and αCD34-NPs were compared to αMSLN-NPs and αStrep-NPs. *=0.0154, **=0.0099, ***=0.0002, ****<0.0001. (D) To determine the specificity of NP binding, experiments were performed without and with pre-blocking the FcRs on PBMCs, followed by incubation of PBMCs with the distinct NP-batches for 15 minutes at 37° C. The data represents the mean±SEM of 2-4 independent experiments, including at least 2 different donors. Statistical significance was calculated using a 2-way ANOVA and Bonferroni's multiple comparison test. Fc-blocked samples were compared to their non-blocked counterpart. (E, F) Kinetics of binding of NPs conjugated to α-gpr56(Cys), α-CD34(Cys) or α-MSLN (Cys) mAbs, conjugated by (E) NHS-ester and (F) maleimide-thiol reaction by CD34+ PBMCs over time (0-120 minutes). The percentage of CD34+ cells that had internalized the fluorescent carriers was determined by flow cytometry. Data represent mean±SEM of 2 independent experiments, using one donor. Statistical significance was calculated using a 2-way ANOVA and Bonferroni's multiple comparison test. agpr56- and αCD34-NPs were compared at each time point to αMSLN-NPs. **=0.006, ***=0.0005, ****=<0.0001. (G) Binding and uptake of NPs conjugated α-gpr56(Cys), α-CD34(Cys), α-MSLN (Cys) or α-Strep (Cys) conjugated by maleimide-thiol reaction by CD34+ PBMCs for 1 hour at 4° C. (binding) or 37° C. (binding and uptake). The data represents the mean±SEM of 2 independent experiments, including 2 donors. Statistical significance was calculated using a 2-way ANOVA and Bonferroni's multiple comparison test. agpr56-, αCD34-NPs and αMSLN were compared at each. (H) Binding of NPs coated with α-gpr56(Cys), α-CD34(Cys), α-MSLN (Cys) or α-Strep (Cys) conjugated by maleimide-thiol reaction by isolated CD34+ blood cells (from PBMCs). CD34+ cells were isolated from PBMCs with the help of CD34+ magnetic beads, and subsequently incubated with 10 (μg/ml of the distinct NP batches). The percentage of CD34+ cells that had bound the fluorescent carriers was determined by flow cytometry.

FIG. 5. α-gpr56-PLGA-PEG-NP and α-CD34-PLGA-PEG-NP are internalized by HSPCs. (A) Human PBMCs or (B) isolated CD34+ cell (from PBMCs) were incubated for 1 hour with NPs coated with α-gpr56(Cys)(top images), α-CD34(Cys)(middle images) and α-MSLN (Cys) (bottom images) (conjugated via maleimide-thiol reaction), washed and subsequently seeded on poly-L-Lysine-coated coverslips for 15 minutes, fixed and stained for CD34 (green) and DAPI (blue). Encapsulated DiD (red). Cells were analysed using a Leica SP5 confocal laser scanning microscope and 63× oil objective. Images represent middle focal planes. Scale bars=10 μm.

FIG. 6. α-gpr56-PLGA-PEG-NP and α-CD34-PLGA-PEG-NP specifically target distinct hematopoietic progenitor populations within blood. (A) Flow cytometric dot plots showing gating and percentages of HSPC populations in peripheral blood. Selectivity of NP uptake was determined within the distinct HSPC subpopulations. PBMCs were incubated for 15 minutes at 37° C. with 20 μg/ml α-gpr56(Cys)-PLGA-PEG-NP, α-CD34(Cys)-PLGA-PEG-NP and α-Strep(Cys)-PLGA-PEG-NP (Maleimide) and subsequently stained with a panel of flow cytometric markers. (Top) CD34+ cells were gated combining CD34, CD45, SSC, and FSC parameters in a sequential Boolean gating strategy following the Clinical and Laboratory Standards Institute (CLSI) H42-A2 approved guideline. Dead cells were excluded by 7-AAD-positive staining. Monocytes and lymphocytes could be gated based on their morphology within the CD45V population. (Middle) Viable CD34+ cells (CD34V) were further gated into a CD34+CD38− population. The CD34+CD38− events were then subdivided further into a multipotent progenitor cell MPP population, characterized as CD34+CD38−CD90−CD45RA−, and a HSC populations characterized as CD34+CD38−CD90+CD45RA−. (B) Viable CD34+ cells subdivided into CD38+CD10+ events were common lymphoid progenitor cells (CLPs). The CD38+CD10− population contained common myeloid progenitor cells (CMPs, CD34+CD38+CD135+CD45RA−), granulocyte macrophage progenitor cells (GMPs, CD34+CD38+CD135+CD45RA+), and megakaryocyte erythroid progenitor cells (MEPs, CD34+CD38+CD135−CD45RA−). (C) Summary flow cytometric analysis of DiD+ PBMC subsets. The percentage of DiD+ cells was analysed in different PBMC subpopulations as gated in A. (D) The data of panel C represents the mean±SEM of 4-7 independent experiments, including at least 3 different donors.

FIG. 7. Selection of high-affinity clones (A) Screening for αCD34 high-affinity clones by flow cytometry. Examples of tested clones. Jurkat cells were incubated with 10 μg/ml primary antibodies (rat Isotype), followed by labelling with α-ratAF647 secondary antibody. (B) Screening for αGpr56 high-affinity clones by flow cytometry. Examples of tested clones. 32D-Gpr56 cells were incubated with 10 μg/ml primary antibodies (rat Isotype), followed by labelling with α-ratAF647 secondary antibody.

FIG. 8. Confocal overview images of α-gpr56-PLGA-PEG-NP and α-CD34-PLGA-PEG-NP targeting HSPCs within PBMCs. (A) Human PBMCs were incubated for 1 hour with NPs coated with α-gpr56(Cys), α-CD34(Cys) and α-MSLN (Cys) (conjugated via maleimide-thiol), washed and subsequently seeded on poly-L-Lysine-coated coverslips for 15 minutes, fixed and stained for CD34 (green) and DAPI (blue). Encapsulated DiD (red). Cells were analysed using a Leica SP5 confocal laser scanning microscope and 63× oil objective. Images represent middle focal planes. Scale bars=100 μm.

FIG. 9. Overview of CD34 antibodies. Top panel shows the affinities of different anti-CD34 antibodies. Bottom panel show the sequence of heavy and light chains of the anti-CD34 antibodies. The red arrow indicates the sequences of the anti CD34 antibody L5F1 used in the targeting experiments. Sequences, from top to bottom, are SEQ ID NOs: 32-67.

FIG. 10. Sequences of Gpr56 antibodies. The sequences of the heavy and light chains of a number of anti Gpr56 antibodies are shown. The red arrow indicated the antibody sequences of anti Gpr56 antibody 3.8g8, which was used in the targeting experiments. Sequences, from top to bottom, are SEQ ID NOs: 68-91.

FIG. 11. Characterization of CRISPR/Cas9-PLGA-NPs. (A) Schematic representation of a CRISPR/Cas9-nanoparticle (NP) made of polylactic-co-glycolic acid (PLGA). The CRISPR-components were encapsulated in form of single guide RNA (sgRNA) and purified Cas9 (S. pyogenes) protein. In addition, the NPs were equipped with a fluorescent dye (acid Cy5). (B) Schematic overview of the CRISPR/Cas9-PLGA-NP synthesis protocol using a double emulsion solvent evaporation method. (C) Representative transmission electron microscopy image of a CRISPR/Cas9-PLGA-NP formulation. Scale bar=2 μm. (D) Representative dynamic light scattering measurement of a CRISPR/Cas9-PLGA-NP formulation. The average size of the NPs was 350-400 nm in diameter. Release kinetic studies of (E) Atto-550-labeled gRNA and (F) Cas9 from CRISPR/Cas9-PLGA-NPs in PBS pH 7.4 at 37° C. Insets represent the kinetic release curve over the first 24 hours. At the indicated time-points, release medium was collected and gRNA and Cas9 levels were quantified by spectrophotometer and nanodrop measurements, respectively. Results show representative release kinetic curves.

FIG. 12. Upregulation of HbF in primary human erythroblasts. Human PBMCs were differentiated towards erythroblasts and on day 8 (start of phase 2) treated with CRISPR/Cas9-PLGA- or control-NPs. (A) Upper panel, representative flow cytometry plots of HbF expression in primary erythroblasts 3 days post treatment with 50 μg/ml or 200 μg/ml CRISPR/Cas9-PLGA-NPs, encapsulating sgRNA shown to induce upregulation of HbF [21], or 200 μg/ml control-NPs encapsulating a scrambled sgRNA sequence. Lower panel, % of NP+(Cy5+) cells. (B) % of β-globin mRNA relative to total levels of β-globin+γ-globin. (C) % of HbF+ cells on day 3, 8 and 14 after treatment with 50, 100 or 200 μg/ml CRISPR/Cas9-PLGA-NPs, or 200 μg/ml control-NPs. (D) Top, portrait of the HBB locus on chromosome 11. A section of the HBG1 promoter region indicating spacer (purple) and protospacer adjacent motif (pink), which specify the site of sgRNA-binding and Cas9 cleavage. The gRNA is complementary to the antisense strand. Green nucleotides indicate the BCL11A binding site. The arrow indicates the predicted Cas9-cleavage site. Bottom, Sanger sequencing trace data from primary erythroblasts 14 days after treatment with 200 μg/ml CRISPR/Cas9-PLGA-NPs or control-NPs. Sequences, from top to bottom, are SEQ ID NOs: 92-94. (E) TIDE analysis on the Sanger sequencing data of the bulk of erythroblasts edited with CRISPR/Cas9-PLGA-NPs.

FIG. 13. Cy5 expression in methylcellulose colonies derived from WT or CRISPR/Cas9-PLGA-NP-treated human CD34+ cells. Colonies were imaged on day 14 with the Odyssey scanner at 700 nm. Cy5=green.

FIG. 14. NP-uptake and gene editing of the HBG promoter region in primary human CD34+ cells. (A) Top, representative pictures of isolated CD34+ cells treated with 100 μg/ml CRISPR/Cas9-PLGA-NPs (Cy5, red) for 1 hour at 37° C., analysed by confocal microscopy. Cells were washed, fixed and stained with DAPI (nucleus=blue). Bottom row, labelling of the cell surface marker CD34 (green) was included. (B) Binding and uptake of CRISPR/Cas9-PLGA-NPs by isolated CD34+ cells for 1 hour at 4° C. (binding) or 37° C. (binding and uptake). The data represents the mean±SEM of 2 independent experiments, including 1 donor. (C) Representative flow cytometry plot of CD34+ cells treated with 200 μg/ml CRISPR/Cas9-PLGA-NPs for 30 min at 37° C. before plating into methylcellulose. (D) Representative images of colonies grown from CD34+WT cells, or CD34+ cells treated with CRISPR/Cas9-PLGA-NPs, imaged with a fluorescent microscope. NPs (green), scale bar=100 μm. (E) Hematopoietic progenitor numbers in WT, CRISPR/Cas9-PLGA-NP- or control-NP-treated isolated CD34+ cells. CFU-C (colony forming unit-culture) per 500 sorted cells is shown, with colony types designated by coloured bars: CFU-G=CFU-granulocyte, CFU-M=macrophage and CFU-GM=CFU-granulocyte, macrophage, CFU-GEMM=CFU-granulocyte, erythroid, macrophage, megakaryocyte, and BFU-E=burst forming unit erythroid. (F) Example TIDE analysis of a BFU-E colony edited with CRISPR/Cas9-PLGA-NPs. (G) TIDE and (H) γ-globin/β-globin mRNA analysis of WT BFU-E colonies, or colonies edited with CRISPR/Cas9-PLGA-NPs or control-NPs. As positive control, the RNP complex was delivered to CD34+ cells by electroporation. Data represent the mean±SEM of 5 independent experiments. All P-values were compared to the respective control cells using Mann-Whitney test. **=<p 0.0065, ****=<p 0.001.

### DETAILED DESCRIPTION

Antibody-conjugated nanoparticles are provided herein, along with methods for using said antibody-conjugated nanoparticles and preferred therapeutic uses for said antibody-conjugated nanoparticles. The invention additionally provides novel antibodies which are suitable for therapeutic use, particularly when conjugated to nanoparticles.

The antibody-conjugated nanoparticles of the present invention comprise an antibody, such as anti-CD34 or anti-Gpr56 antibody, conjugated to a nanoparticle, such as a PLGA-PEG-nanoparticle. The nanoparticle preferably comprises a payload, such as a therapeutic payload, for example a gene editing payload.

The Antibody

In preferred embodiments, the antibody of the antibody-conjugated nanoparticle is a monoclonal antibody.

Preferably, the antibody is a human antibody. As used herein, a human antibody is any fully human antibody, i.e. a monoclonal antibody comprising no sequences derived from a non-human species. Such human antibodies may be obtained from transgenic rodents, such as mice, comprising human and non-human, such as endogenous rodent, immunoglobulin genes. The antibodies derived from such transgenic rodents may be chimeric antibodies comprising human variable regions and rodent constant regions. Human antibodies may be derived from chimeric antibodies by the use of recombinant DNA technology to replace the rodent constant region with a human constant region, thereby providing a fully human antibody having human variable and constant regions. Human antibodies may also be obtained from a human, for example from an isolated human B cell.

In an embodiment, the antibody is a humanised antibody. As used herein, a humanised antibody is an antibody derived from a non-human species that has been modified to increase the similarity to antibodies derived from humans. A humanised antibody may comprise a human constant region and a chimeric variable region, such as a chimeric human-mouse constant region. Particularly, a humanised antibody may comprise non-human complementarity-determining regions (CDRs), for example mouse heavy and light CDRs. A humanised antibody may be obtained by a process of CDR grafting, in which a monoclonal mouse antibody is found and the DNA sequences of the CDRs of said antibody are identified and isolated. Recombinant DNA technology may then be used to replace the CDR sequences of a fully human antibody with the mouse CDR sequences, thereby providing the sequence of a humanised antibody having the desired mouse CDRs. Further modifications may be made in the region of the CDR sequences in order to obtain an optimal balance of minimal immunogenicity while maintaining the desired binding and antigen recognition characteristics.

Alternatively, the antibody may be a chimeric antibody, such as a chimeric human-mouse antibody comprising human variable regions and mouse constant regions. The antibody may also be a fully non-human antibody, for example a fully mouse antibody, a fully rabbit antibody, or a fully rat antibody.

In an embodiment, the antibody is any antibody that is not immunogenic, such as any antibody that does not illicit an immune response or does not illicit a substantial immune response in humans.

In embodiments, the antibody may be a full-length antibody comprising Fab and Fc regions, for example the antibody may be an IgG1, IgG2, IgG3, IgG4, IgA, or IgE antibody. The antibody may also be an antibody fragment, such as a Fab fragment. Preferably, the antibody is an IgG1 antibody. In an embodiment, the constant region of the antibody is mutated to reduce or remove the constant region effector function(s). In an embodiment, the antibody is an IgG1 antibody containing an Asn297Ala mutation, said mutation removing effector functions.

In one embodiment, the antibody is a heavy chain-only antibody (HCAb). HCAbs consist of two heavy chains and lack the two light chains usually present in tetrameric antibodies. Preferably, the HCAb is a human or humanised HCAb. Methods for making such human or humanised HCAbs are described in, e.g., WO 2006/008548, WO 2007/096779, WO2010/109165, and WO 2014/141192. Alternatively, the HCAb may be a camelid HCAb, such as a llama, camel, or alpaca HCAb.

Recently, methods for the production of heavy-chain-only antibodies in transgenic mammals have been developed (see WO 2006/008548). Functional heavy chain-only antibody of potentially any class (IgM, IgG, IgD, IgA or IgE) and derived from any mammal (including man) can be produced from transgenic mammals (preferably mice) as a result of antigen challenge.

Heavy chain-only monoclonal antibodies can be recovered from B-cells of the spleen and lymph nodes by standard cloning technology (WO 2006/008548, WO 2007/096779, WO2010/109165, WO 2014/141192), standard hybridoma technology or recovered from B-cell mRNA by phage display technology (Ward et al., (1989) Nature, 341, 544-546). Heavy chain-only monoclonal antibodies can also be directly cloned from antibody-producing cells (e.g. plasma cells or memory B cells) into mammalian cells (WO 2010/109165; Drabek et al. (2016) Frontiers in Immunology 7:619). Heavy chain-only antibodies derived from camelids or transgenic animals are of high affinity. Sequence analysis of expressed heavy chain-only mRNA demonstrates that diversity results primarily from a combination of VDJ rearrangement and somatic hypermutation, whether produced in camelids or transgenic animals, supports this observation (De Genst et al., (2005) J. Biol. Chem., 280, 14114-14121, and WO 2006/008548).

An important and common feature of natural camelid and human VH(VHH) regions is that each region binds as a monomer with no dependency on dimerisation with a V_{L} region for optimal solubility and binding affinity. These features have previously been recognised as particularly suited to the production of blocking agents and tissue penetration agents (for review see Holliger, P. & Hudson, P. J. (2005) Nature Biotechnology 23, 1126-1136) or the generation of multivalent heavy chain only antibodies (WO 2006/008548).

Recent progress using transgenic rodents have shown that Heavy Chain Only Antibodies can also be obtained when using human VH regions are used as part of an immunoglobulin locus (WO 2006/008548, WO 2007/096779, WO2010/109165, WO 2014/141192).

Due to the antigen-binding specificity of antibody molecules, the nanoparticle conjugated to the antibody is targeted to a particular target cell. In an embodiment, the antibody is specific to an antigen which is expressed by or on the surface of the target cell. In an embodiment, the antibody is specific to an antigen which is only expressed by or on the surface of the target cell, i.e. no other cell types express the antigen to which the antibody is specific.

In an embodiment, the antibody targets a cell-surface antigen, such as a cell-surface protein. In an embodiment, the antibody targets a cell-surface protein that is preferably specific to a targeted cell, for example a cell which is a therapeutic target. The antibody thereby targets the nanoparticle to said target cell, and the payload is preferably delivered exclusively to the target cell. In an embodiment, the target cell may be a tumour cell, such as a cancerous tumour cell, and optionally the cell-surface protein is a growth factor receptor. In an embodiment, the target cell is a stem cell. In an embodiment, the target cell is a progenitor cell. In a preferred embodiment, the target cell is a hematopoietic stem cell. Most preferably, the targeted cell-surface protein is only expressed on the targeted cell.

In some embodiments, the antibody targets a cancer cell surface antigen. Accordingly, in some embodiments, an anti-cancer cell surface antigen antibody conjugated to a nanoparticle comprising a therapeutic payload (e.g. a gene editing payload) is provided. In some embodiments, the anti-cancer cell surface antigen antibody-conjugated nanoparticle is for use in treating a cancer that expresses the cancer cell surface antigen.

In some embodiments, the antibody targets CD33. Accordingly, in some embodiments, an anti-CD33 antibody conjugated to a nanoparticle comprising a therapeutic payload (e.g. a gene editing payload) is provided. In some embodiments, the anti-CD33 antibody-conjugated nanoparticle is for use in treating a CD33+ myeloid leukaemia (e.g. acute myeloid leukaemia).

In some embodiments, the antibody targets CD30. Accordingly, in some embodiments, an anti-CD30 antibody conjugated to a nanoparticle comprising a therapeutic payload (e.g. a gene editing payload) is provided. In some embodiments, the anti-CD30 antibody-conjugated nanoparticle is for use in treating a CD30+ lymphoma (e.g. Hodgkin lymphoma or anaplastic large cell lymphoma).

In some embodiments, the antibody targets CD22. Accordingly, in some embodiments, an anti-CD22 antibody conjugated to a nanoparticle comprising a therapeutic payload (e.g. a gene editing payload) is provided. In some embodiments, the anti-CD22 antibody-conjugated nanoparticle is for use in treating a CD22+ leukaemia (e.g. an acute lymphoblastic leukaemia, such as B-cell precursor acute lymphoblastic leukaemia).

In some embodiments, the antibody targets CD79b. Accordingly, in some embodiments, an anti-CD79b antibody conjugated to a nanoparticle comprising a therapeutic payload (e.g. a gene editing payload) is provided. In some embodiments, the anti-CD79b antibody-conjugated nanoparticle is for use in treating a CD79b+ lymphoma (e.g. non-Hodgkin lymphoma, such as diffuse large B cell lymphoma).

In some embodiments, the antibody targets Nectin-4. Accordingly, in some embodiments, an anti-Nectin-4 antibody conjugated to a nanoparticle comprising a therapeutic payload (e.g. a gene editing payload) is provided. In some embodiments, the anti-Nectin-4 antibody-conjugated nanoparticle is for use in treating Nectin-4+ bladder cancer.

HER2, EGFR, EGFRvIII, cMET, FGFR-2 and FGFR-3 are target antigens associated with driver oncogenes.

In some embodiments, the antibody targets HER2. Accordingly, in some embodiments, an anti-HER2 antibody conjugated to a nanoparticle comprising a therapeutic payload (e.g. a gene editing payload) is provided. In some embodiments, the anti-HER2 antibody-conjugated nanoparticle is for use in treating HER2+ solid tumours (e.g. breast cancer, oesphogeal cancer or stomach cancer).

In some embodiments, the antibody targets EGFR. Accordingly, in some embodiments, an anti-EGFR antibody conjugated to a nanoparticle comprising a therapeutic payload (e.g. a gene editing payload) is provided. In some embodiments, the anti-EGFR antibody-conjugated nanoparticle is for use in treating a EGFR+ solid tumour (e.g. breast cancer, such as triple negative breast cancer, head and neck squamous cell carcinoma or non-small cell lung cancer).

In some embodiments, the antibody targets EGFRvIII. Accordingly, in some embodiments, an anti-EGFRvIII antibody conjugated to a nanoparticle comprising a therapeutic payload (e.g. a gene editing payload) is provided. In some embodiments, the anti-EGFRvIII antibody-conjugated nanoparticle is for use in treating an EGFRvIII+ glioma (e.g. glioblastoma).

In some embodiments, the antibody targets cMET. Accordingly, in some embodiments, an anti-cMET antibody conjugated to a nanoparticle comprising a therapeutic payload (e.g. a gene editing payload) is provided. In some embodiments, the anti-cMET antibody-conjugated nanoparticle is for use in treating a cMET+ solid tumour.

In some embodiments, the antibody targets FGFR2. Accordingly, in some embodiments, an anti-FGFR2 antibody conjugated to a nanoparticle comprising a therapeutic payload (e.g. a gene editing payload) is provided. In some embodiments, the anti-FGFR2 antibody-conjugated nanoparticle is for use in treating a FGFR2+ solid tumour.

In some embodiments, the antibody targets FGFR3. Accordingly, in some embodiments, an anti-FGFR3 antibody conjugated to a nanoparticle comprising a therapeutic payload (e.g. a gene editing payload) is provided. In some embodiments, the anti-FGFR3 antibody-conjugated nanoparticle is for use in treating a FGFR3+ solid tumour.

AXL, HER3, CD166, CEACAM5, GPNMB, mesothelin, LIV1A, tissue factor (TF), CD71, CD228, FRα, NaPi2b, Trop-2, PSMA, CD70, STEAP1, P Cadherin, SLITRK6, LAMP1, CA9, GPR20 and CLDN18.2 are antigens that are overexpressed in some cancer cells.

In some embodiments, the antibody targets AXL. Accordingly, in some embodiments, an anti-AXL antibody conjugated to a nanoparticle comprising a therapeutic payload (e.g. a gene editing payload) is provided. In some embodiments, the anti-AXL antibody-conjugated nanoparticle is for use in treating an AXL+ solid tumour (e.g. ovarian cancer, cervical cancer, endometrial cancer, non-small cell lung cancer, thyroid cancer, melanoma or sarcoma).

In some embodiments, the antibody targets HER3. Accordingly, in some embodiments, an anti-HER3 antibody conjugated to a nanoparticle comprising a therapeutic payload (e.g. a gene editing payload) is provided. In some embodiments, the anti-HER3 antibody-conjugated nanoparticle is for use in treating HER3+ solid tumours (e.g. breast cancer or non-small cell lung cancer).

In some embodiments, the antibody targets CD166. Accordingly, in some embodiments, an anti-CD166 antibody conjugated to a nanoparticle comprising a therapeutic payload (e.g. a gene editing payload) is provided. In some embodiments, the anti-CD166 antibody-conjugated nanoparticle is for use in treating CD166+ solid tumours (e.g. breast cancer, ovarian cancer, head and neck squamous cell carcinoma or non-small cell lung cancer).

In some embodiments, the antibody targets CEACAM5. Accordingly, in some embodiments, an anti-CEACAM5 antibody conjugated to a nanoparticle comprising a therapeutic payload (e.g. a gene editing payload) is provided. In some embodiments, the anti-CEACAM5 antibody-conjugated nanoparticle is for use in treating CEACAM5+ solid tumours (e.g. colorectal cancer, (non-squamous) non-small cell lung cancer, small cell lung cancer, or gastric cancer).

In some embodiments, the antibody targets GPNMB. Accordingly, in some embodiments, an anti-GPNMB antibody conjugated to a nanoparticle comprising a therapeutic payload (e.g. a gene editing payload) is provided. In some embodiments, the anti-GPNMB antibody-conjugated nanoparticle is for use in treating GPNMB+ solid tumours (e.g. breast cancer, such as triple negative breast cancer, (squamous) non-small cell lung cancer, (uveal) melanoma, or osteosarcoma).

In some embodiments, the antibody targets mesothelin. Accordingly, in some embodiments, an anti-mesothelin antibody conjugated to a nanoparticle comprising a therapeutic payload (e.g. a gene editing payload) is provided. In some embodiments, the anti-mesothelin antibody-conjugated nanoparticle is for use in treating mesothelin+ solid tumours (e.g. mesothelioma, (non-small cell) lung cancer, ovarian cancer or pancreatic cancer). Accordingly, in one embodiment, an antibody-conjugated nanoparticle comprising a cytotoxic payload, wherein the antibody binds to the membrane-bound form of mesothelin is provided, optionally for use in treating a pancreatic tumour.

In some embodiments, the antibody targets LIV1A. Accordingly, in some embodiments, an anti-LIV1A antibody conjugated to a nanoparticle comprising a therapeutic payload (e.g. a gene editing payload) is provided. In some embodiments, the anti-LIV1A antibody-conjugated nanoparticle is for use in treating LIV1A+ solid tumours (e.g. breast cancer, such as triple negative breast cancer).

In some embodiments, the antibody targets tissue factor (TF). Accordingly, in some embodiments, an anti-TF antibody conjugated to a nanoparticle comprising a therapeutic payload (e.g. a gene editing payload) is provided. In some embodiments, the anti-TF antibody-conjugated nanoparticle is for use in treating TF+ solid tumours (e.g. breast cancer, such as triple negative breast cancer).

In some embodiments, the antibody targets CD71. Accordingly, in some embodiments, an anti-CD71 antibody conjugated to a nanoparticle comprising a therapeutic payload (e.g. a gene editing payload) is provided. In some embodiments, the anti-CD71 antibody-conjugated nanoparticle is for use in treating CD71+ solid tumours (e.g. (non-small cell) lung cancer, head and neck squamous cell carcinoma, or ovarian carcinoma).

In some embodiments, the antibody targets CD228. Accordingly, in some embodiments, an anti-CD228 antibody conjugated to a nanoparticle comprising a therapeutic payload (e.g. a gene editing payload) is provided. In some embodiments, the anti-CD228 antibody-conjugated nanoparticle is for use in treating CD228+ solid tumours (e.g. (cutaneous) melanoma, (pleural) mesothelioma, breast cancer, (non-small cell) lung cancer, colorectal cancer, or pancreatic ductal adenocarcinoma).

In some embodiments, the antibody targets FRα. Accordingly, in some embodiments, an anti-FRa antibody conjugated to a nanoparticle comprising a therapeutic payload (e.g. a gene editing payload) is provided. In some embodiments, the anti-FRα antibody-conjugated nanoparticle is for use in treating FRα+ solid tumours (e.g. ovarian carcinoma, fallopian tube cancer, or (primary) peritoneal carcinoma).

In some embodiments, the antibody targets NaPi2b. Accordingly, in some embodiments, an anti-NaPi2b antibody conjugated to a nanoparticle comprising a therapeutic payload (e.g. a gene editing payload) is provided. In some embodiments, the anti-NaPi2b antibody-conjugated nanoparticle is for use in treating NaPi2b+ solid tumours (e.g. ovarian carcinoma or (non-small cell) lung cancer).

In some embodiments, the antibody targets Trop-2. Accordingly, in some embodiments, an anti-Trop-2 antibody conjugated to a nanoparticle comprising a therapeutic payload (e.g. a gene editing payload) is provided. In some embodiments, the anti-Trop-2 antibody-conjugated nanoparticle is for use in treating Trop-2+ solid tumours (e.g. breast cancer, such as triple negative breast cancer or hormone receptor positive breast cancer, ovarian carcinoma, gastric cancer, pancreatic cancer, (non-small cell) lung cancer, or urothelial carcinoma).

In some embodiments, the antibody targets PSMA. Accordingly, in some embodiments, an anti-PSMA antibody conjugated to a nanoparticle comprising a therapeutic payload (e.g. a gene editing payload) is provided. In some embodiments, the anti-PSMA antibody-conjugated nanoparticle is for use in treating PSMA+ solid tumours (e.g. prostate carcinoma or glioblastoma).

In some embodiments, the antibody targets CD70. Accordingly, in some embodiments, an anti-CD70 antibody conjugated to a nanoparticle comprising a therapeutic payload (e.g. a gene editing payload) is provided. In some embodiments, the anti-CD70 antibody-conjugated nanoparticle is for use in treating CD70+ solid tumours (e.g. renal cell carcinoma or non-Hodgkin lymphoma).

In some embodiments, the antibody targets STEAP1. Accordingly, in some embodiments, an anti-STEAP1 antibody conjugated to a nanoparticle comprising a therapeutic payload (e.g. a gene editing payload) is provided. In some embodiments, the anti-STEAP1 antibody-conjugated nanoparticle is for use in treating STEAP1+ solid tumours (e.g. prostate cancer, such as metastatic castration-resistant prostate cancer).

In some embodiments, the antibody targets P Cadherin. Accordingly, in some embodiments, an anti-P Cadherin antibody conjugated to a nanoparticle comprising a therapeutic payload (e.g. a gene editing payload) is provided. In some embodiments, the anti-P Cadherin antibody-conjugated nanoparticle is for use in treating P Cadherin+ solid tumours (e.g. breast cancer, such as triple negative breast cancer, oesophageal cancer or head and neck squamous cell carcinoma).

In some embodiments, the antibody targets SLITRK6. Accordingly, in some embodiments, an anti-SLITRK6 antibody conjugated to a nanoparticle comprising a therapeutic payload (e.g. a gene editing payload) is provided. In some embodiments, the anti-SLITRK6 antibody-conjugated nanoparticle is for use in treating SLITRK6+ solid tumours (e.g. urothelial carcinoma).

In some embodiments, the antibody targets LAMP1. Accordingly, in some embodiments, an anti-LAMP1 antibody conjugated to a nanoparticle comprising a therapeutic payload (e.g. a gene editing payload) is provided. In some embodiments, the anti-LAMP1 antibody-conjugated nanoparticle is for use in treating LAMP1+ solid tumours.

In some embodiments, the antibody targets CA9. Accordingly, in some embodiments, an anti-CA9 antibody conjugated to a nanoparticle comprising a therapeutic payload (e.g. a gene editing payload) is provided. In some embodiments, the anti-CA9 antibody-conjugated nanoparticle is for use in treating CA9+ solid tumours.

In some embodiments, the antibody targets GPR20. Accordingly, in some embodiments, an anti-GPR20 antibody conjugated to a nanoparticle comprising a therapeutic payload (e.g. a gene editing payload) is provided. In some embodiments, the anti-GPR20 antibody-conjugated nanoparticle is for use in treating GPR20+ solid tumours (e.g. gastrointestinal stromal tumour).

In some embodiments, the antibody targets CLDN18.2. Accordingly, in some embodiments, an anti-CLDN18.2 antibody conjugated to a nanoparticle comprising a therapeutic payload (e.g. a gene editing payload) is provided. In some embodiments, the anti-GPR20 antibody-conjugated nanoparticle is for use in treating CLDN18.2+ solid tumours (e.g. gastric or pancreatic tumours).

Leucine-rich repeat containing 15 (LRRC15), FAPα, ANTXR1, TM4SF1, CD25, CD205, B7-H3 and HLA-DR are target antigens in some tumour microenvironments.

In some embodiments, the antibody targets LRRC15. Accordingly, in some embodiments, an anti-LRRC15 antibody conjugated to a nanoparticle comprising a therapeutic payload (e.g. a gene editing payload) is provided. In some embodiments, the anti-LRRC15 antibody-conjugated nanoparticle is for use in treating a solid tumour (e.g. a sarcoma, such as undifferentiated pleomorphic sarcoma, head and neck squamous cell carcinoma or breast cancer).

In some embodiments, the antibody targets FAPα. Accordingly, in some embodiments, an anti-FAPα antibody conjugated to a nanoparticle comprising a therapeutic payload (e.g. a gene editing payload) is provided. In some embodiments, the anti-FAPα antibody-conjugated nanoparticle is for use in treating a solid tumour (e.g. (non-small cell) lung cancer).

In some embodiments, the antibody targets ANTXR1. Accordingly, in some embodiments, an anti-ANTXR1 antibody conjugated to a nanoparticle comprising a therapeutic payload (e.g. a gene editing payload) is provided. In some embodiments, the anti-ANTXR1 antibody-conjugated nanoparticle is for use in treating a solid tumour.

In some embodiments, the antibody targets TM4SF1. Accordingly, in some embodiments, an anti-TM4SF1 antibody conjugated to a nanoparticle comprising a therapeutic payload (e.g. a gene editing payload) is provided. In some embodiments, the anti-TM4SF1 antibody-conjugated nanoparticle is for use in treating a solid tumour.

In some embodiments, the antibody targets CD25. Accordingly, in some embodiments, an anti-CD25 antibody conjugated to a nanoparticle comprising a therapeutic payload (e.g. a gene editing payload) is provided. In some embodiments, the anti-CD25 antibody-conjugated nanoparticle is for use in treating a solid tumour (e.g. a solid tumour with CD25+ Treg cells) or a CD25+ hematologic tumour.

In some embodiments, the antibody targets CD205. Accordingly, in some embodiments, an anti-CD205 antibody conjugated to a nanoparticle comprising a therapeutic payload (e.g. a gene editing payload) is provided. In some embodiments, the anti-CD205 antibody-conjugated nanoparticle is for use in treating a solid tumour (e.g. non-Hodgkin lymphoma).

In some embodiments, the antibody targets B7-H3. Accordingly, in some embodiments, an anti-B7-H3 antibody conjugated to a nanoparticle comprising a therapeutic payload (e.g. a gene editing payload) is provided. In some embodiments, the anti-B7-H3 antibody-conjugated nanoparticle is for use in treating a solid tumour.

In some embodiments, the antibody targets HLA-DR. Accordingly, in some embodiments, an anti-HLA-DR antibody conjugated to a nanoparticle comprising a therapeutic payload (e.g. a gene editing payload) is provided. In some embodiments, the anti-HLA-DR antibody-conjugated nanoparticle is for use in treating cancer (e.g. a hematologic tumour or melanoma).

Delta-like 1 homolog protein (DLK-1), Delta-like ligand 3 (DLL3), Ephrin-A4 (EFNA4), PTK7, ROR1, 5T4 and KAAG1 are target antigens expressed by some stem cells.

In some embodiments, the antibody targets DLK-1. Accordingly, in some embodiments, an anti-DLK-1 antibody conjugated to a nanoparticle comprising a therapeutic payload (e.g. a gene editing payload) is provided. In some embodiments, the anti-DLK1 antibody-conjugated nanoparticle is for use in treating a solid tumour (e.g. liver cancer).

In some embodiments, the antibody targets DLL-3. Accordingly, in some embodiments, an anti-DLL-3 antibody conjugated to a nanoparticle comprising a therapeutic payload (e.g. a gene editing payload) is provided. In some embodiments, the anti-DLL-3 antibody-conjugated nanoparticle is for use in treating a solid tumour (e.g. (small cell) lung cancer).

In some embodiments, the antibody targets EFNA4. Accordingly, in some embodiments, an anti-EFNA4 antibody conjugated to a nanoparticle comprising a therapeutic payload (e.g. a gene editing payload) is provided. In some embodiments, the anti-EFNA4 antibody-conjugated nanoparticle is for use in treating a solid tumour.

In some embodiments, the antibody targets PTK7. Accordingly, in some embodiments, an anti-PTK7 antibody conjugated to a nanoparticle comprising a therapeutic payload (e.g. a gene editing payload) is provided. In some embodiments, the anti-PTK7 antibody-conjugated nanoparticle is for use in treating a solid tumour (e.g. a breast cancer, such as triple negative breast cancer, ovarian carcinoma or (non-small cell) lung cancer).

In some embodiments, the antibody targets ROR1. Accordingly, in some embodiments, an anti-ROR1 antibody conjugated to a nanoparticle comprising a therapeutic payload (e.g. a gene editing payload) is provided. In some embodiments, the anti-ROR1 antibody-conjugated nanoparticle is for use in treating a solid tumour (e.g. a breast cancer, such as triple negative breast cancer).

In some embodiments, the antibody targets 5T4. Accordingly, in some embodiments, an anti-5T4 antibody conjugated to a nanoparticle comprising a therapeutic payload (e.g. a gene editing payload) is provided. In some embodiments, the anti-5T4 antibody-conjugated nanoparticle is for use in treating a solid tumour.

In some embodiments, the antibody targets KAAG1. Accordingly, in some embodiments, an anti-KAAG1 antibody conjugated to a nanoparticle comprising a therapeutic payload (e.g. a gene editing payload) is provided. In some embodiments, the anti-KAAG1 antibody-conjugated nanoparticle is for use in treating a solid tumour.

In the context of cancer therapy, the therapeutic payload may be a gene editing payload that is capable of knocking out or disrupting a gene essential for cancer cell survival or replication. For example, in the treatment of CML, the gene editing payload may be capable of knocking out or disrupting BCR, ABL1, SOS1, GRB2 or GAB2. For example, in the treatment of lymphoma, the gene editing payload may be capable of knocking out or disrupting EBF1, POU2AF1, PAX5, MEF2B, or CCND3. In alternative embodiment, the therapeutic payload is a cytotoxic payload. For example, in one embodiment, an anti-mesothelin antibody conjugated to a nanoparticle comprising a cytotoxic payload is for use in treating a mesothelin+ cancer, such as a mesothelin+ pancreatic tumour.

In some embodiments, the antibody targets a hematopoietic stem cell (HSC) surface antigen. Accordingly, in some embodiments, an anti-HSC cell surface antigen antibody conjugated to a nanoparticle comprising a therapeutic payload (e.g. a gene editing payload) is provided. In some embodiments, the anti-HSC cell surface antigen antibody-conjugated nanoparticle is for use in treating a genetic blood disorder (e.g. sickle cell disease, hemoglobinopathy, or thalassemia). In some embodiments, the anti-HSC cell surface antigen antibody-conjugated nanoparticle is for use in treating a congenital immunodeficiency (e.g. severe combined immunodeficiency or hypogammoglobulinemia). In some embodiments, the anti-HSC cell surface antigen antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

CD34, Gpr56, Gpr97, CD49, CD49f, CD90, CD117 and endomucin are typically expressed on hematopoietic stem cells.

In some embodiments, the antibody targets CD34. Accordingly, in some embodiments, an anti-CD34 antibody conjugated to a nanoparticle comprising a therapeutic payload (e.g. a gene editing payload) is provided. In some embodiments, the anti-CD34 antibody-conjugated nanoparticle is for use in treating a genetic blood disorder (e.g. sickle cell disease, hemoglobinopathy, or thalassemia). In some embodiments, the anti-CD34 antibody-conjugated nanoparticle is for use in treating a congenital immunodeficiency (e.g. severe combined immunodeficiency or hypogammoglobulinemia). In some embodiments, the anti-CD34 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets Gpr56. Accordingly, in some embodiments, an anti-Gpr56 antibody conjugated to a nanoparticle comprising a therapeutic payload (e.g. a gene editing payload) is provided. In some embodiments, the anti-Gpr56 antibody-conjugated nanoparticle is for use in treating a genetic blood disorder (e.g. sickle cell disease, hemoglobinopathy, or thalassemia). In some embodiments, the anti-Gpr56 antibody-conjugated nanoparticle is for use in treating a congenital immunodeficiency (e.g. severe combined immunodeficiency or hypogammoglobulinemia). In some embodiments, the anti-Gpr56 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia). In some embodiments, the anti-Gpr56 antibody-conjugated nanoparticle is for use in treating a Gpr56+ solid tumour (e.g. a brain cancer, such as a glioma).

In some embodiments, the antibody targets Gpr97. Accordingly, in some embodiments, an anti-Gpr97 antibody conjugated to a nanoparticle comprising a therapeutic payload (e.g. a gene editing payload) is provided. In some embodiments, the anti-Gpr97 antibody-conjugated nanoparticle is for use in treating a genetic blood disorder (e.g. sickle cell disease, hemoglobinopathy, or thalassemia). In some embodiments, the anti-Gpr97 antibody-conjugated nanoparticle is for use in treating a congenital immunodeficiency (e.g. severe combined immunodeficiency or hypogammoglobulinemia). In some embodiments, the anti-Gpr97 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets CD49. Accordingly, in some embodiments, an anti-CD49 antibody conjugated to a nanoparticle comprising a therapeutic payload (e.g. a gene editing payload) is provided. In some embodiments, the anti-CD49 antibody-conjugated nanoparticle is for use in treating a genetic blood disorder (e.g. sickle cell disease, hemoglobinopathy, or thalassemia). In some embodiments, the anti-CD49 antibody-conjugated nanoparticle is for use in treating a congenital immunodeficiency (e.g. severe combined immunodeficiency or hypogammoglobulinemia). In some embodiments, the anti-CD49 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets CD49f. Accordingly, in some embodiments, an anti-CD49f antibody conjugated to a nanoparticle comprising a therapeutic payload (e.g. a gene editing payload) is provided. In some embodiments, the anti-CD49f antibody-conjugated nanoparticle is for use in treating a genetic blood disorder (e.g. sickle cell disease, hemoglobinopathy, or thalassemia). In some embodiments, the anti-CD49f antibody-conjugated nanoparticle is for use in treating a congenital immunodeficiency (e.g. severe combined immunodeficiency or hypogammoglobulinemia). In some embodiments, the anti-CD49f antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets CD90. Accordingly, in some embodiments, an anti-CD90 antibody conjugated to a nanoparticle comprising a therapeutic payload (e.g. a gene editing payload) is provided. In some embodiments, the anti-CD90 antibody-conjugated nanoparticle is for use in treating a genetic blood disorder (e.g. sickle cell disease, hemoglobinopathy, or thalassemia). In some embodiments, the anti-CD90 antibody-conjugated nanoparticle is for use in treating a congenital immunodeficiency (e.g. severe combined immunodeficiency or hypogammoglobulinemia). In some embodiments, the anti-CD90 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets CD117. Accordingly, in some embodiments, an anti-CD117 antibody conjugated to a nanoparticle comprising a therapeutic payload (e.g. a gene editing payload) is provided. In some embodiments, the anti-CD117 antibody-conjugated nanoparticle is for use in treating a genetic blood disorder (e.g. sickle cell disease, hemoglobinopathy, or thalassemia). In some embodiments, the anti-CD117 antibody-conjugated nanoparticle is for use in treating a congenital immunodeficiency (e.g. severe combined immunodeficiency or hypogammoglobulinemia). In some embodiments, the anti-CD117 antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody targets endomucin. Accordingly, in some embodiments, an anti-endomucin antibody conjugated to a nanoparticle comprising a therapeutic payload (e.g. a gene editing payload) is provided. In some embodiments, the anti-endomucin antibody-conjugated nanoparticle is for use in treating a genetic blood disorder (e.g. sickle cell disease, hemoglobinopathy, or thalassemia). In some embodiments, the anti-endomucin antibody-conjugated nanoparticle is for use in treating a congenital immunodeficiency (e.g. severe combined immunodeficiency or hypogammoglobulinemia). In some embodiments, the anti-endomucin antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody is a bispecific antibody that targets two of the following antigens: CD34, Gpr56, Gpr97, CD49, CD49f, CD90, CD117 and endomucin. In some embodiments, the bispecific antibody is conjugated to a nanoparticle comprising a therapeutic payload (e.g. a gene editing payload). In some embodiments, the bispecific antibody-conjugated nanoparticle is for use in treating a genetic blood disorder (e.g. sickle cell disease, hemoglobinopathy, or thalassemia). In some embodiments, the bispecific antibody-conjugated nanoparticle is for use in treating a congenital immunodeficiency (e.g. severe combined immunodeficiency or hypogammoglobulinemia). In some embodiments, the bispecific antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In some embodiments, the antibody is a multispecific antibody that targets three or more (e.g. four, five or six) of the following antigens: CD34, Gpr56, Gpr97, CD49, CD49f, CD90, CD117 and endomucin. In some embodiments, the multispecific antibody is conjugated to a nanoparticle comprising a therapeutic payload (e.g. a gene editing payload). In some embodiments, the multispecific antibody-conjugated nanoparticle is for use in treating a genetic blood disorder (e.g. sickle cell disease, hemoglobinopathy, or thalassemia). In some embodiments, the multispecific antibody-conjugated nanoparticle is for use in treating a congenital immunodeficiency (e.g. severe combined immunodeficiency or hypogammoglobulinemia). In some embodiments, the multispecific antibody-conjugated nanoparticle is for use in treating a cancer (e.g. a hematologic or lymphoid cancer, such as multiple myeloma, Hodgkin lymphoma, non-Hodgkin lymphoma, acute myeloid leukaemia, chronic myeloid leukaemia, or chronic lymphocytic leukaemia).

In the context of treating genetic blood disorders or congenital immunodeficiency, the therapeutic payload may be a gene editing payload that is capable of correcting a genetic defect that contributes to the genetic blood disorder or congenital immunodeficiency.

In the context of treating cancer, the therapeutic payload may be a gene editing payload that is capable of knocking out or disrupting a gene essential for (cancer) cell survival or replication (e.g. a gene encoding a polymerase or polymerase subunit). In an alternative embodiment, the therapeutic payload is a cytotoxic payload. For example, in one embodiment, an anti-Gpr56 antibody conjugated to a nanoparticle comprising a cytotoxic payload is for use in treating a Gpr56+ solid tumour, such as a Gpr56+ brain cancer (e.g. Gpr56+ glioma), optionally wherein the antibody-conjugated nanoparticle is delivered directly through the skull to the tumour.

In a preferred embodiment, the antibody is an anti-CD34 antibody. In an alternative preferred embodiment, the antibody is an anti-Gpr56 antibody.

In embodiments, the antibody is bispecific. For example, the antibody may be a bispecific anti-CD34 and anti-Gpr56 antibody. The antibody may alternatively be a multispecific antibody.

CD34 is a cell surface antigen on multipotent progenitor (MPP) cells, multipotent lymphoid progenitor (MLP) cells, common myeloid progenitor (CMP) cells, megakaryocyte-erythroid progenitor (MEP) cells, colony-forming unit—megakaryocytes (CFU-Mk), burst-forming unit—erythroid (BFU-E), granulocyte-macrophage progenitor (GMP) and common lymphoid progenitor (CLP) cells.

CD45RA is a cell surface antigen on MLP, GMP and CLP cells.

CD38 is a cell surface antigen on CMP, BFU-E and GMP cells. It is also present at low levels on MEP cells and some CLP cells.

In some embodiments, the antibody targets CD34. Accordingly, in some embodiments, an anti-CD34 antibody conjugated to a nanoparticle comprising a therapeutic payload (e.g. a gene editing payload) is provided. In some embodiments, the anti-CD34 antibody-conjugated nanoparticle is for use in treating a hematologic cancer (e.g. leukaemia).

In some embodiments, the antibody targets CD45RA. Accordingly, in some embodiments, an anti-CD45RA antibody conjugated to a nanoparticle comprising a therapeutic payload (e.g. a gene editing payload) is provided. In some embodiments, the anti-CD45RA antibody-conjugated nanoparticle is for use in treating a hematologic cancer (e.g. leukaemia).

In some embodiments, the antibody targets CD38. Accordingly, in some embodiments, an anti-CD38 antibody conjugated to a nanoparticle comprising a therapeutic payload (e.g. a gene editing payload) is provided. In some embodiments, the anti-CD38 antibody-conjugated nanoparticle is for use in treating a hematologic cancer (e.g. leukaemia).

In some embodiments, the antibody is a bispecific antibody that targets CD34 and CD45RA, CD34 and CD38 or CD45RA and CD38. In some embodiments, the bispecific antibody is conjugated to a nanoparticle comprising a therapeutic payload (e.g. a gene editing payload). In some embodiments, the antibody-conjugated nanoparticle is for use in treating a hematologic cancer (e.g. leukaemia).

In some embodiments, the antibody is a trispecific antibody that targets CD34, CD38 and CD45RA. In some embodiments, the trispecific antibody is conjugated to a nanoparticle comprising a therapeutic payload (e.g. a gene editing payload). In some embodiments, the antibody-conjugated nanoparticle is for use in treating a hematologic cancer (e.g. leukaemia).

In the context of treating hematologic cancer (e.g. leukaemia), the therapeutic payload may be a gene editing payload that is capable of knocking out or disrupting a gene essential for (cancer) cell survival or replication (e.g. a gene encoding a polymerase or polymerase subunit).

In some embodiments, the antibody targets a T cell surface antigen. Accordingly, in some embodiments, an anti-T cell surface antigen antibody conjugated to a nanoparticle comprising a therapeutic payload (e.g. a gene editing payload) is provided. In some embodiments, the anti-T cell surface antigen antibody-conjugated nanoparticle is for use in treating a cancer or pathogenic disease by increasing a T cell response specific for the cancer or pathogen. In some embodiments, the anti-T cell surface antigen antibody-conjugated nanoparticle is for use in treating cancer by increasing the activity of neoantigen-specific T cells. In some embodiments, the gene editing payload is capable of knocking out or disrupting PD-1.

CD4, CD8, CD3, CTLA4, TCR, TCRα and TCRβ are T cell surface antigens.

In some embodiments, the antibody targets CD4. Accordingly, in some embodiments, an anti-CD4 antibody conjugated to a nanoparticle comprising a therapeutic payload (e.g. a gene editing payload) is provided. In some embodiments, the anti-CD4 antibody-conjugated nanoparticle is for use in treating a cancer or pathogenic disease by increasing a T cell response specific for the cancer or pathogen. In some embodiments, the anti-CD4 surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets CD8. Accordingly, in some embodiments, an anti-CD8 antibody conjugated to a nanoparticle comprising a therapeutic payload (e.g. a gene editing payload) is provided. In some embodiments, the anti-CD8 antibody-conjugated nanoparticle is for use in treating a cancer or pathogenic disease by increasing a T cell response specific for the cancer or pathogen. In some embodiments, the anti-CD4 surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets CD3. Accordingly, in some embodiments, an anti-CD3 antibody conjugated to a nanoparticle comprising a therapeutic payload (e.g. a gene editing payload) is provided. In some embodiments, the anti-CD3 antibody-conjugated nanoparticle is for use in treating a cancer or pathogenic disease by increasing a T cell response specific for the cancer or pathogen. In some embodiments, the anti-CD3 surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets CTLA4. Accordingly, in some embodiments, an anti-CTLA4 antibody conjugated to a nanoparticle comprising a cytotoxic payload is provided. In some embodiments, an anti-CTLA4 antibody conjugated to a nanoparticle comprising a gene editing payload is provided, optionally wherein the gene editing payload is capable of disrupting or knocking out a gene essential for T cell survival or replication (e.g. a gene encoding a polymerase or polymerase subunit). In some embodiments, the anti-CTLA4 antibody-conjugated nanoparticle is for use in treating a cancer or pathogenic disease by increasing a cytotoxic T cell response specific for the cancer or pathogen by decreasing the number of T regulatory cells in the tumor. In some embodiments, the anti-CTLA4 surface antigen antibody-conjugated nanoparticle is for use in treating cancer by increasing the activity of neoantigen-specific (cytotoxic) T cells. In some embodiments, the gene editing payload is capable of knocking out or disrupting PD-1.

In some embodiments, the antibody targets TCR. Accordingly, in some embodiments, an anti-TCR antibody conjugated to a nanoparticle comprising a therapeutic payload (e.g. a gene editing payload) is provided. In some embodiments, the anti-TCR antibody-conjugated nanoparticle is for use in treating a cancer or pathogenic disease by increasing a T cell response specific for the cancer or pathogen. In some embodiments, the anti-TCR surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets TCRα. Accordingly, in some embodiments, an anti-TCRα antibody conjugated to a nanoparticle comprising a therapeutic payload (e.g. a gene editing payload) is provided. In some embodiments, the anti-TCRα antibody-conjugated nanoparticle is for use in treating a cancer or pathogenic disease by increasing a T cell response specific for the cancer or pathogen. In some embodiments, the anti-TCRα surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets TCRβ. Accordingly, in some embodiments, an anti-TCRβ antibody conjugated to a nanoparticle comprising a therapeutic payload (e.g. a gene editing payload) is provided. In some embodiments, the anti-TCRβ antibody-conjugated nanoparticle is for use in treating a cancer or pathogenic disease by increasing a T cell response specific for the cancer or pathogen. In some embodiments, the anti-TCRβ surface antigen antibody-conjugated nanoparticle is for use in treating cancer by increasing the activity of neoantigen-specific T cells. In some embodiments, the gene editing payload is capable of knocking out or disrupting PD-1.

In some embodiments, the antibody targets a dendritic cell surface antigen. Accordingly, in some embodiments, an anti-dendritic cell surface antigen antibody conjugated to a nanoparticle comprising a therapeutic payload (e.g. a gene editing payload) is provided. In some embodiments, the anti-dendritic cell surface antigen antibody-conjugated nanoparticle is for use in preventing transplant rejection. In some embodiments, the gene editing payload is capable of knocking out or disrupting CD40.

HLA-DR and CD40 are surface antigens on dendritic cells.CD1c, Dectin 1, Dectin 2, CD141, CLEC9A and XCR1 are surface antigens on classical dendritic cells. CD303, CD304 and CD123 are surface antigens on plasmacytoid dendritic cells. CD14, CD209 and Factor XIIIA are surface antigens of CD14+ monocyte-related dendritic cells. CD16, CX3CR1 and SLAN are surface antigens of CD16+ monocyte-related dendritic cells. CD1c is a surface antigen of inflammatory dendritic cells. In some embodiments, the antibody targets HLA-DR. Accordingly, in some embodiments, an anti-HLA-DR antibody conjugated to a nanoparticle comprising a therapeutic payload (e.g. a gene editing payload) is provided. In some embodiments, the anti-HLA-DR antibody-conjugated nanoparticle is for use in preventing transplant rejection. In some embodiments, the gene editing payload is capable of knocking out or disrupting CD40.

In some embodiments, the antibody targets CD40. Accordingly, in some embodiments, an anti-CD40 antibody conjugated to a nanoparticle comprising a therapeutic payload (e.g. a gene editing payload) is provided. In some embodiments, the anti-CD40 antibody-conjugated nanoparticle is for use in preventing transplant rejection. In some embodiments, the gene editing payload is capable of knocking out or disrupting CD40.

In some embodiments, the antibody targets one of the following antigens: CD1c, Dectin 1, Dectin 2, CD141, CLEC9A and XCR1. In some embodiments, the antibody is conjugated to a nanoparticle comprising a therapeutic payload (e.g. a gene editing payload). In some embodiments, the antibody-conjugated nanoparticle is for use in preventing transplant rejection. In some embodiments, the gene editing payload is capable of knocking out or disrupting CD40.

In some embodiments, the antibody targets one of the following antigens: CD303, CD304 and CD123. In some embodiments, the antibody is conjugated to a nanoparticle comprising a therapeutic payload (e.g. a gene editing payload). In some embodiments, the antibody-conjugated nanoparticle is for use in preventing transplant rejection. In some embodiments, the gene editing payload is capable of knocking out or disrupting CD40.

In some embodiments, the antibody targets one of the following antigens: CD14, CD209 and Factor XIIIA. In some embodiments, the antibody is conjugated to a nanoparticle comprising a therapeutic payload (e.g. a gene editing payload). In some embodiments, the antibody-conjugated nanoparticle is for use in preventing transplant rejection. In some embodiments, the gene editing payload is capable of knocking out or disrupting CD40.

In some embodiments, the antibody targets one of the following antigens: CD16, CX3CR1 and SLAN. In some embodiments, the antibody is conjugated to a nanoparticle comprising a therapeutic payload (e.g. a gene editing payload). In some embodiments, the antibody-conjugated nanoparticle is for use in preventing transplant rejection. In some embodiments, the gene editing payload is capable of knocking out or disrupting CD40.

In some embodiments, the antibody targets CD1c. In some embodiments, the antibody is conjugated to a nanoparticle comprising a therapeutic payload (e.g. a gene editing payload). In some embodiments, the antibody-conjugated nanoparticle is for use in preventing transplant rejection. In some embodiments, the gene editing payload is capable of knocking out or disrupting CD40.

In some embodiments, the antibody is a bispecific or multispecific HCAb, such as a bispecific or multi-specific human or humanised HCAb. In some embodiments, the antibody is a bispecific or multi-specific antibody derived from one or more HCAb antibodies and one or more H2L2 antibodies, for example the bispecific antibody may comprise one light chain and two heavy chains.

The invention also provides an anti-CD34 antibody comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 1; an HCDR2 comprising the amino acid sequence of SEQ ID NO: 2; and an HCDR3 comprising the amino acid sequence of SEQ ID NO: 3; and the VL comprises an LCDR1 comprising the amino acid sequence of SEQ ID NO: 4; an LCDR2 comprising the amino acid sequence of SEQ ID NO: 5; and an LCDR3 comprising the amino acid sequence of SEQ ID NO: 6.

The anti-CD34 antibody of the invention may comprise a heavy chain variable region comprising the amino acid sequence of SEQ ID NO: 7. The anti-CD34 antibody of the invention may comprise a light chain variable region comprising the amino acid sequence of SEQ ID NO: 8. The anti-CD34 antibody of the invention may comprise a heavy chain variable region comprising the amino acid sequence of SEQ ID NO: 7 and a light chain variable region comprising the amino acid sequence of SEQ ID NO: 8.

The invention further provides an anti-CD34 antibody that binds to the same epitope as an antibody comprising a heavy chain variable region of the amino acid sequence of SEQ ID NO: 7 and a light chain variable region of the amino acid sequence of SEQ ID NO: 8.

The invention further provides an anti-CD34 antibody that binds to the same epitope as a heavy chain-only antibody comprising a heavy chain variable region of the amino acid sequence of SEQ ID NO: 7.

The invention further provides an anti-CD34 antibody that competes for binding to CD34 with an antibody comprising a heavy chain variable region of the amino acid sequence of SEQ ID NO: 7 and a light chain variable region of the amino acid sequence of SEQ ID NO: 8.

The invention further provides an anti-CD34 antibody that competes for binding to CD34 with a heavy chain-only antibody comprising a heavy chain variable region of the amino acid sequence of SEQ ID NO: 7.

In some embodiments, the antibody comprises a heavy chain variable region of the amino acid sequence of SEQ ID NO: 7.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 7.

In some embodiments, the antibody comprises a light chain variable region of the amino acid sequence of SEQ ID NO: 8.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 8.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 7 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 8.

The invention further provides an anti-CD34 antibody comprising complementarity determining regions (CDRs) with:
i. a sequence that is at least 90% identical to SEQ ID NO: 1 for CDR1 of the heavy chain;
ii. a sequence that is at least 90% identical to SEQ ID NO: 2 for CDR2 of the heavy chain;
iii. a sequence that is at least 90% identical to SEQ ID NO: 3 for CDR3 of the heavy chain;
iv. a sequence that is at least 90% identical to SEQ ID NO: 4 for CDR1 of the light chain;
v. a sequence that is at least 90% identical to SEQ ID NO: 5 for CDR2 of the light chain; and
vi. a sequence that is at least 90% identical to SEQ ID NO: 6 for CDR3 of the light chain,
   wherein the antibody competes for binding to CD34 with an antibody comprising a heavy chain variable region of the amino acid sequence of SEQ ID NO: 7 and a light chain variable region of the amino acid sequence of SEQ ID NO: 8.

The invention further provides an anti-CD34 antibody comprising complementarity determining regions (CDRs) with:
i. a sequence that is at least 95% identical to SEQ ID NO: 1 for CDR1 of the heavy chain;
ii. a sequence that is at least 95% identical to SEQ ID NO: 2 for CDR2 of the heavy chain;
iii. a sequence that is at least 95% identical to SEQ ID NO: 3 for CDR3 of the heavy chain;
iv. a sequence that is at least 95% identical to SEQ ID NO: 4 for CDR1 of the light chain;
v. a sequence that is at least 95% identical to SEQ ID NO: 5 for CDR2 of the light chain; and
vi. a sequence that is at least 95% identical to SEQ ID NO: 6 for CDR3 of the light chain,
   wherein the antibody competes for binding to CD34 with an antibody comprising a heavy chain variable region of the amino acid sequence of SEQ ID NO: 7 and a light chain variable region of the amino acid sequence of SEQ ID NO: 8.

The invention also provides an anti-Gpr56 antibody, comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 9; an HCDR2 comprising the amino acid sequence of SEQ ID NO: 10; and an HCDR3 comprising the amino acid sequence of SEQ ID NO: 11; and the VL comprises an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

The anti-Gpr56 antibody of the invention may comprise a heavy chain variable region comprising the amino acid sequence of SEQ ID NO: 15. The anti-Gpr56 antibody of the invention may comprise a light chain variable region comprising the amino acid sequence of SEQ ID NO: 16. The anti-Gpr56 antibody of the invention may comprise a heavy chain variable region comprising the amino acid sequence of SEQ ID NO: 15 and a light chain variable region comprising the amino acid sequence of SEQ ID NO: 16.

The invention further provides an anti-Gpr56 antibody that binds to the same epitope as an antibody comprising a heavy chain variable region of the amino acid sequence of SEQ ID NO: 15 and a light chain variable region of the amino acid sequence of SEQ ID NO: 16.

The invention further provides an anti-Gpr56 antibody that binds to the same epitope as a heavy chain-only antibody comprising a heavy chain variable region of the amino acid sequence of SEQ ID NO: 15.

The invention further provides an anti-Gpr56 antibody that competes for binding to Gpr56 with an antibody comprising a heavy chain variable region of the amino acid sequence of SEQ ID NO: 15 and a light chain variable region of the amino acid sequence of SEQ ID NO: 16.

The invention further provides an anti-Gpr56 antibody that competes for binding to Gpr56 with a heavy chain-only antibody comprising a heavy chain variable region of the amino acid sequence of SEQ ID NO: 15.

In some embodiments, the antibody comprises a heavy chain variable region of the amino acid sequence of SEQ ID NO: 15.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 15.

In some embodiments, the antibody comprises a light chain variable region of the amino acid sequence of SEQ ID NO: 16.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 16.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 15 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 16.

The invention further provides an anti-Gpr56 antibody comprising complementarity determining regions (CDRs) with:
i. a sequence that is at least 90% identical to SEQ ID NO: 9 for CDR1 of the heavy chain;
ii. a sequence that is at least 90% identical to SEQ ID NO: 10 for CDR2 of the heavy chain;
iii. a sequence that is at least 90% identical to SEQ ID NO: 11 for CDR3 of the heavy chain;
iv. a sequence that is at least 90% identical to SEQ ID NO: 12 for CDR1 of the light chain;
v. a sequence that is at least 90% identical to SEQ ID NO: 13 for CDR2 of the light chain; and
vi. a sequence that is at least 90% identical to SEQ ID NO: 14 for CDR3 of the light chain,
   wherein the antibody competes for binding to Gpr56 with an antibody comprising a heavy chain variable region of the amino acid sequence of SEQ ID NO: 15 and a light chain variable region of the amino acid sequence of SEQ ID NO: 16.

The invention further provides an anti-Gpr56 antibody comprising complementarity determining regions (CDRs) with:
i. a sequence that is at least 95% identical to SEQ ID NO: 9 for CDR1 of the heavy chain;
ii. a sequence that is at least 95% identical to SEQ ID NO: 10 for CDR2 of the heavy chain;
iii. a sequence that is at least 95% identical to SEQ ID NO: 11 for CDR3 of the heavy chain;
iv. a sequence that is at least 95% identical to SEQ ID NO: 12 for CDR1 of the light chain;
v. a sequence that is at least 95% identical to SEQ ID NO: 13 for CDR2 of the light chain; and
vi. a sequence that is at least 95% identical to SEQ ID NO: 14 for CDR3 of the light chain,
   wherein the antibody competes for binding to Gpr56 with an antibody comprising a heavy chain variable region of the amino acid sequence of SEQ ID NO: 15 and a light chain variable region of the amino acid sequence of SEQ ID NO: 16.

The section below specifies features that are combinable with each of the previous embodiments, as appropriate.

In some embodiments, the antibody binds to the target antigen (e.g. CD34 or Gpr56) with a K_{D} of 10⁻⁷ M or less, 10⁻⁸ M or less, 10⁻⁹ M or less, or 10⁻¹⁰ M or less. In some embodiments, antibody binding affinity is determined using an Octet® RED96 system (ForteBio, Inc.). For example, a Flag-tagged S1 domain or a Flag-tagged S2 domain may be immobilized to an anti-Flag biosensor and incubated with varying concentrations of the antibody in solution, binding data are then collected. In some embodiments, antibody binding affinity is determined by surface plasmon resonance.

In some embodiments, whether a test antibody competes with a reference antibody for binding to a target antigen (e.g. CD34 or Gpr56) is determined using an in vitro binding competition assay. For example, a Flag-tagged antigen (e.g. CD34 or Gpr56) may be immobilized to an anti-Flag biosensor, the association of the reference antibody to the immobilized Flag-tagged antigen is then measured (e.g. using the Octet® RED96 system, ForteBio, Inc.) and then the degree of additional binding is assessed by exposing the immobilized Flag-tagged antigen to the test antibody in the presence of the reference antibody.

The anti-CD34 or anti-Gpr56 antibody of the invention additionally comprises a constant region. Preferably, the anti-CD34 or anti-Gpr56 antibody of the invention comprises human a constant region, such as a human IgG1 constant region.

In preferred embodiments, antibody-conjugate of the invention comprises the anti-CD34 or anti-Gpr56 antibody of the invention.

The Nanoparticle

In an embodiment, the nanoparticle of the antibody-conjugated nanoparticle has a diameter of between 1 and 500 nm. Preferably, the nanoparticle has a diameter of between 150 and 400 nm, such as 200 to 400 nm. The diameter of a nanoparticle may be measured by any suitable method, such as dynamic light scattering, nanoparticle tracking analysis, transmission electron microscopy, scanning electron microscopy, atomic force microscopy, photo correlation spectroscopy, x-ray diffraction, or time of flight mass spectroscopy. Preferably, the diameter of a nanoparticle is measured by dynamic light scattering or nanoparticle tracking analysis. The diameter of the nanoparticle is preferably measured when the nanoparticle comprises the payload, most preferably when the nanoparticle has encapsulated the payload such that the payload is in the interior of the nanoparticle.

Preferably, the nanoparticles are biodegradable. Additionally, the nanoparticles are preferably non-toxic, more preferably non-toxic to a human patient.

In an embodiment, the nanoparticles have a negative zeta potential. In an embodiment, the zeta potential is between −30 and 0 mV, such as between −25 and −5 mV.

In an embodiment, the nanoparticle comprises one or more of: chitosan, alginate, xanthan gum, cellulose, poly(lactic-co-glycolic acid), polyethylene glycol, poly(propylene glycol), poly(aspartic acid), poly(lactic acid). In an embodiment, the nanoparticle is one of: a liposome, a polymeric micelle, a dendrimer [1]. In an embodiment, the nanoparticle comprises biodegradable polymers.

The nanoparticle may be surface modified, for example a nanoparticle may be coated with polyethylene glycol (PEG).

The nanoparticle may be synthesised using any suitable method, for example solvent extraction, microfluidic nanoparticle production, dialysis, solution casting, polycarbonate membrane extrusion, high pressure homogenisation, reversed phase evaporation, sonication, or lipid film hydration sonication extrusion (see for example [2]).

In an embodiment, the nanoparticle comprises poly(lactic-co-glycolic acid) (PLGA). In an embodiment, the nanoparticle comprises polyethylene glycol (PEG). In a preferred embodiment, the nanoparticle comprises PLGA and PEG. More preferably, the nanoparticle consists essentially of PLGA and PEG, and optionally the nanoparticle consists of PLGA and PEG. In an embodiment, the nanoparticle consists of a PLGA core that is surface coated in PEG.

A nanoparticle comprising or consisting of PLGA and/or PEG may be synthesised by any suitable method, such as emulsification-evaporation, salting out, nanoprecipitation, or using microfluidics (see, e.g., Danhier 2012 Journal of Controlled Release 161(2):505-522).

The nanoparticle is preferably capable of being taken up by a cell and releases the payload inside the cell, thereby delivering the payload directly to the intracellular space. In an embodiment, the antibody-conjugated nanoparticle is taken up by a cell by pinocytosis. Particularly when the nanoparticle has a diameter less than 500 nm, the nanoparticle is taken up by a cell by pinocytosis [3].

In an embodiment, the antibody-conjugated nanoparticle is taken up by a cell by phagocytosis. In an embodiment, the nanoparticle is configured to release the payload in a pH-dependent manner, for example the payload is released when the pH is below 7, 6.5, 6, 5.5, or 5 (see for example [4]).

The Payload

The antibody-conjugated nanoparticles of the invention comprise a payload.

Preferably, the payload is encapsulated by the nanoparticle such that the payload is in the interior of the nanoparticle. In an embodiment, the payload is encapsulated within the interior of a PLGA core of the nanoparticle. Encapsulation within a nanoparticle protects the payload from premature degradation. Additionally or alternatively, the payload may be conjugated to the nanoparticle.

In an embodiment, the payload is a therapeutic payload, which may be any payload, which may be administered to a patient in need thereof, which treats or ameliorates the symptoms of one or more diseases. In an embodiment, the payload is a medicament, such as a medicament for cancer therapy, for example a chemotherapy drug. In an embodiment, the payload is a toxin, such as an alkylating agent. A toxin as used herein is any molecule which causes the cell to which the payload is delivered to die.

In an embodiment, the payload, optionally a therapeutic payload, comprises at least one of the following: a drug, a protein, RNA, DNA, an imaging agent.

In preferred embodiments, the payload is a gene editing payload. A gene editing payload is any payload that is configured to edit, i.e. change, a patient's genome or a cell's genome in any manner. The gene editing preferably treats or ameliorates the symptoms of a disorder, such as a genetic disorder, from which the patient is suffering. In a preferred embodiment, the gene editing payload changes the DNA sequence of a patient's genome or a cell's genome. Alternatively, the gene editing payload changes patient's genome or cell's genome by making epigenetic changes, by regulating transcription of a gene, or by arresting DNA replication.

The gene editing payload preferably comprises a nuclease, most preferably an endonuclease, which is configured to cause a single or double strand break at a specific or non-specific site in the patient's genome or cell genome. A specific site may be a specific gene within the patient's genome or cell genome or a specific sequence within the genome. A non-specific site may be any site within the genome, for example the nuclease may cause a double strand break at a random site in the genome.

In an embodiment, the payload comprises at least one of: a meganuclease, a zinc-finger nuclease (ZFN), a transcription activator-like effector nuclease (TALEN), a clustered regularly interspaced palindromic repeats (CRISPR) nuclease (see for example [5]).

Alternatively, the payload comprises mRNA or DNA encoding at least one of: a meganuclease, a zinc-finger nuclease (ZFN), a transcription activator-like effector nuclease (TALEN), a clustered regularly interspaced palindromic repeats (CRISPR) nuclease.

In preferred embodiments, the payload comprises a CRISPR nuclease, and optionally the CRISPR nuclease is selected from the following: CRISPR-associated protein 9 (Cas9), CRISPR associated protein 12a (Cas12a, also known as Cpf1), CRISPR associated protein 13 (Cas13), CRISPR associated protein 12b (Cas12b), CRISPR associated protein X (CasX, also known as Cas12e), CRISPR associated protein Y (CasY), CRISPR associated protein 14a (Cas14a). A suitable CRISPR nuclease may be selected on the basis of, for example, selectivity requirements and/or size requirements, for example the nuclease must be small enough to be encapsulated by the nanoparticle (see for example bitesizebio.com/46146/crispr-nucleases-the-ultimate-guide/ and synthego.com/guide/how-to-use-crispr/cas9-nuclease-variants). When Cas9 is the nuclease, the Cas9 may be derived from Streptococcus pyogenes (SpCas9), Staphylococcus aureus (SaCas9), Streptococcus thermophilus (StCas9), Neisseria meningitidis (NmCas9), Francisella novicida (FnCas9), Streptococcus canis (ScCas9), or Campylobacter jejuni (CjCas9). The CRISPR nuclease may be derived from a naturally occurring CRISPR nuclease or may be an engineered CRISPR nuclease. In particular, the gene editing payload may comprise an engineered CRISPR nuclease selected from the following: High-fidelity SpCas9 (SpCas9-HF1, eSpCas9-1.1), SpCas9 with relaxed PAM (SpCas9-NG), SpCas9 nickase (Cas9n), or Nuclease-dead SpCas9 (dCas9). Nuclease-dead SpCas9 is preferably used when the gene editing payload is configured to change a patient's genome or cell genome by making epigenetic changes or by regulating transcription of a gene [6]. Nuclease-dead SpCas9 may also be used to arrest DNA replication in a cell [7], for example to arrest DNA replication in a tumour cell, thereby interrupting the process of cell division and slowing the growth of a tumour. When SpCas9 nickase is used, preferably the gene editing payload comprises two versions of the nickase in order to generate a double stranded break, wherein each nickase causes a single stranded break at the target site.

Preferably, the nuclease is SpCas9.

Preferably, the nuclease is fused to one or more nuclear localisation signal sequences.

Most preferably, the nuclease is Cas9 fused to one or more nuclear localisation signal (NLS) sequences (e.g. Cas9 fused to three NLS sequences).

In an embodiment where the payload comprises a CRISPR nuclease, the payload further comprises a CRISPR RNA (crRNA) and a trans-activating crRNA (tracrRNA). The crRNA and tracrRNA together act as a guide for the CRISPR nuclease (cr:tracrRNA guide).

In an alternative embodiment where the payload comprises a CRISPR nuclease, the payload further comprises a single guide RNA (gRNA). A gRNA is used by the CRISPR nuclease to recognize target DNA, and optionally comprises a short crRNA sequence fused to a scaffold tracrRNA sequence. The CRISPR nuclease and gRNA together form a CRISPR ribonucleoprotein complex. In an embodiment, the gRNA is between 17 and 24 bp in length, such as 17 bp, 18 bp, 19 bp, 20 bp, 21 bp, 22 bp, 23 bp, or 24 bp in length. In an embodiment, the gRNA is between 19 and 21 bp, such as 20 bp in length.

The cr:tracrRNAs or gRNA is complementary to the target site such that, when the cr:tracrRNAs or gRNA is in a CRISPR ribonucleoprotein complex with a CRISPR nuclease, the CRISPR nuclease creates a double stranded break at the target site. The target site must precede the protospacer adjacent motif (PAM), which is a short DNA sequence usually between 2 and 6 base pairs in length that is upstream of the DNA region targeted for cleavage by the CRISPR ribonucleoprotein complex. The PAM is required for the CRISPR nuclease to cleave DNA; the CRISPR nuclease may therefore alternatively or additionally be selected on the basis of the PAM requirements of a nuclease, i.e. a nuclease may be selected if it has a PAM sequence that is found in the target site.

In some embodiments, the gene editing payload additionally comprises a donor DNA molecule that contains a template capable of recombining with the patient's genome or cell's genome at the double strand break site. For example, the donor DNA molecule may be double stranded and may be incorporated into the patient's genome or cell's genome by homologous recombination. Alternatively, the donor DNA molecule may be single stranded, and the donor DNA molecule acts as a template for homology-directed repair at the double strand break site. In these embodiments, the gene editing payload is capable of “knocking-in” a gene, for example replacing a disease-causing faulty gene with the wild-type gene, thus restoring normal function of that gene.

In alternative embodiments, the gene editing payload does not comprise a donor DNA molecule and the double strand break is repaired by a cellular repair mechanism causing sequence modifications at the site that generate frame-shift or deficiency mutations at the targeted locus, for example any mechanism by which indels are created, such as non-homologous end joining or microhomology-mediated end joining. Such frame-shift or deficiency mutations cause loss of function at the double-strand break site. In these embodiments, the gene editing payload is capable of “knocking-out” a gene, for example an oncogene may be knocked out.

In an embodiment, the gene editing payload is configured to edit one or more genes or gene loci associated with a disease or disorder, such as a genetic disease or disorder, cancer, or a non-cancerous tumour. When the disease or disorder is a genetic disease or disorder, the gene editing payload may be configured to edit the disease-causing gene or gene locus. In this embodiment, the gene editing payload may be configured to correct the disease-causing gene or gene locus by homologous recombination with a donor DNA strand which comprises the sequence or a partial sequence of the wild-type gene or gene locus. It will be understood that the wild-type gene or gene locus is any non-disease-causing sequence of the gene or gene locus, particularly any sequence of the gene or gene locus which does not result in a disease phenotype.

When the disease or disorder is a cancer, or non-cancerous tumour, the gene editing payload may be configured to cause double strand breaks in one or more genes or gene loci which are essential for cell survival, such as any gene or gene locus in [8]. For, example a gene encoding a polymerase or polymerase subunit.

In an embodiment, the gene editing payload is configured to edit any one or more of the following genes or gene loci in the patient's genome or cell genome: γ-globin, cystic fibrosis transmembrane conductance regulator (CFTR), dystrophin, keratin 5, keratin 14, desmoplakin, plakophilin-1, plakoglobin, plectin, dystonin, exophilin 5, TGM5, laminin subunit alpha-3, laminin subunit beta-3, laminin subunit gamma-2, collagen XVII, integrin alpha-6, CD49d, integrin alpha-3, collagen alpha-1(VII), fermitin family homolog 1, ferrochelatase, Fanconi anaemia complementation group A, B, C, D1, D2, E, F, G, I, J, L, M, N, P, or S, RAD51 homolog C, DNA repair endonuclease XPF, FMR1, frataxin, galactose-1-phosphate uridylytransferase, galactokinase, UDP-glucose-4-epimerase, PRNP, ATP-binding cassette sub-family A member 12, Factor VIII, uroporphyrinogen Ill decarboxylase, huntingtin, fibroblast growth factor receptor 3, tumour protein p53, myostatin, RAG1, RAG2, collagen type 1 alpha 1, collagen type 1 alpha 2, polycystin 1, polycystin 2, protein C, protein S, haemoglobin beta, haemoglobin alpha, hexosaminidase A, fibroblast growth factor receptor 3.

In preferred embodiments, the gene editing payload is configured to edit the γ-globin gene locus in the patient's genome or cell's genome. Preferably, the γ-globin promoter is edited such that recruitment of BCL11A is inhibited. BCL11A is a powerful repressor of the γ-globin gene, and thus inhibition of this binding alleviates the repression of the γ-globin gene, and mimics the naturally occurring hereditary persistence of fetal haemoglobin (HPFH) mutation. Targeting this region can thus cause persistent reactivation of fetal haemoglobin in a cell. In this embodiment, the gene editing payload preferably comprises a CRISPR nuclease, such as Cas9, optionally SpCas9, and a gRNA having the sequence: CTT GTC AAG GCT ATT GGT CA (SEQ ID NO: 17) [9]. These embodiments are particularly preferred when the antibody is an anti-CD34 or anti-Gpr56 antibody. In alterative embodiments, the gene editing payload is configured to edit the haemoglobin beta gene locus in the patient's genome or cell's genome. In such embodiments, the payload preferably additionally comprises a donor DNA strand comprising the sequence or a partial sequence of the wild-type haemoglobin beta gene locus. Thus, the payload is configured to correct the haemoglobin beta gene locus.

Conjugation & Methods of Manufacture

In embodiments of the antibody-conjugated nanoparticle, the nanoparticle is preferably conjugated to the antibody at the antibody Fc region, i.e. the antibody constant region.

In an embodiment, the nanoparticle and antibody are conjugated such that the antibody is randomly oriented on the surface of the nanoparticle. In a more preferred embodiment, the nanoparticle and antibody are conjugated such that the antibody is oriented with the antigen binding site facing away from the nanoparticle core, allowing optimal engagement with target molecules.

In an embodiment, the nanoparticle is conjugated to the antibody via an ester bond to an amino group on the antibody. In an embodiment, the amino group is from a lysine residue in the antibody Fc region. In an embodiment, the conjugation is via a NHS-ester reaction.

In an embodiment, the nanoparticle is conjugated to the antibody via a thioether bond to a thiol group on the antibody. In an embodiment, the amino group is from a cysteine residue in the antibody Fc region. In an embodiment, the cysteine residue is a C-terminal cysteine, i.e. the cysteine residue is at the C-terminal of the Fc region. In an embodiment, the cysteine residue is near the C-terminal of the Fc region, such as anywhere within 20 residues of the C-terminal, such as within 15, 10, 5, 4, 3, or 2 residues of the C-terminal. In an embodiment, the conjugation is via a maleimide-thiol reaction.

In an embodiment, the nanoparticle is conjugated to the antibody in a site-specific manner, i.e. the nanoparticle is conjugated to a specific residue of the antibody, for example the C-terminal residue of the Fc region.

In an embodiment, the nanoparticle is conjugated to one or more antibody molecules. In an embodiment, the nanoparticle is conjugated to 2 or more antibody molecules, such 3, 4, 5, 6, 7, 8, 9, 10, 15, 20, 25, 30, 35, or 40 or more antibody molecules. In an embodiment, the nanoparticle is conjugated to a plurality of antibody molecules.

In an embodiment, the nanoparticle is conjugated to antibody molecules targeting one antigen, for example the nanoparticle may only be conjugated to anti-CD34 or anti-Gpr56 antibody molecules. In an alternative embodiment, the nanoparticle is conjugated to antibody molecules targeting one or more different antigens, for example the nanoparticle may be conjugated to anti-CD34 and anti-Gpr56 antibody molecules. In an embodiment, the nanoparticle is conjugated to antibody molecules targeting 2, 3, 4, 5, 6, 7, 8, 9, or 10 or more different antigens. In this embodiment, the antigens are preferably from the same target cell such that the nanoparticle will be targeted to only one cell type. The nanoparticle may also or alternatively be conjugated to one or more bispecific or multispecific antibodies.

In a preferred embodiment, the nanoparticle is conjugated to one or more monoclonal antibody molecules. In this embodiment, each antibody molecule is the same, i.e. each antibody molecule conjugated to the nanoparticle shares the same sequence.

In an embodiment, the invention provides a method of making an antibody conjugate, comprising:
modifying an antibody heavy chain coding sequence to introduce a cysteine residue at or near the C-terminal end of the heavy chain constant region;
producing a modified antibody from the modified sequence, wherein the modified antibody comprises a cysteine residue at or near the C-terminal end of the heavy chain constant region, wherein said cysteine residue has a free thiol group that is not covalently bonded to another cysteine residue;
obtaining a poly(lactic-co-glycolic acid)(PLGA)-nanoparticle comprising PEG; and
conjugating the nanoparticle to the antibody via a site-specific maleimide linkage, wherein the cysteine residue at or near the C-terminal end of the heavy chain constant region is covalently bonded to one of the one or more PEG groups of the nanoparticle.

In an embodiment, the obtained nanoparticle further comprises an encapsulated payload. The payload may be any payload as described herein, most preferably a gene editing payload.

In an embodiment, the obtained nanoparticle further comprises poly(lactic-co-glycolic acid) (PLGA).

In an embodiment, the nanoparticle is obtained by a method comprising: dissolving PLGA in dichloromethane (DCM); adding a payload to the PLGA-DCM mixture and emulsifying, optionally under sonication; adding the emulsified mixture dropwise to an aqueous phase comprising polyvinyl acetate (PVA) and sonicating the solution; and washing and recovering a nanoparticle comprising PLGA and the encapsulated payload.

In an embodiment, the nanoparticle is freeze dried. In an embodiment, the nanoparticle is rehydrated before conjugation to the antibody.

Alternatively or in addition, the nanoparticle may be conjugated to the antibody using click chemistry. For example, the nanoparticle may be conjugated to the antibody using any one or more of the following reactions: copper(I)-catalyzed azide-alkyne cycloaddition; strain-promoted azide-alkyne cycloaddition; strain-promoted alkyne-nitrone cycloaddition; alkene and azide [3+2] cycloaddition; alkene and tetrazine inverse-demand Diels-Alder; alkene and tetrazole photoclick reaction.

In some embodiments, the nanoparticle is conjugated to the antibody by sortase-mediated transpeptidation (see, e.g. Popp et al. Current Protocols in Protein Science 56(1):15.3.1-15.3.9).

Pharmaceutical Compositions

The invention further provides pharmaceutical compositions comprising an antibody and/or antibody-conjugated nanoparticle of the invention.

In an embodiment, the pharmaceutical composition comprises an anti-CD34 antibody comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 1; an HCDR2 comprising the amino acid sequence of SEQ ID NO: 2; and an HCDR3 comprising the amino acid sequence of SEQ ID NO: 3; and the VL comprises an LCDR1 comprising the amino acid sequence of SEQ ID NO: 4; an LCDR2 comprising the amino acid sequence of SEQ ID NO: 5; and an LCDR3 comprising the amino acid sequence of SEQ ID NO: 6.

In an embodiment, the pharmaceutical composition comprises an anti-Gpr56 antibody comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 9; an HCDR2 comprising the amino acid sequence of SEQ ID NO: 10; and an HCDR3 comprising the amino acid sequence of SEQ ID NO: 11; and the VL comprises an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

In an embodiment, the pharmaceutical composition comprises an antibody conjugate comprising an anti-CD34 antibody and a nanoparticle conjugated to the antibody, wherein the nanoparticle comprises a payload. In some embodiments, the antibody is a HCAb.

In an embodiment, the pharmaceutical composition comprises an antibody conjugate comprising an anti-Gpr56 antibody and a nanoparticle conjugated to the antibody, wherein the nanoparticle comprises a payload. In some embodiments, the antibody is a HCAb.

In an embodiment, the pharmaceutical composition comprises an antibody conjugate comprising an antibody and a nanoparticle conjugated to the antibody, wherein the nanoparticle comprises a gene editing payload. In some embodiments, the antibody is a HCAb.

In an embodiment, the pharmaceutical composition further comprises one or more pharmaceutically acceptable excipients. The one or more pharmaceutically acceptable excipients may be selected from the list comprising: a solvent, an emulsifier, a carrier, an anti-adherent, a binder, a coating, a colour, a dye, a preservative, a buffering agent, a tonicity agent, an antioxidant, a chelating agent, a complexing agent, a solubilizing agent, a flocculating agent, a suspending agent, a wetting agent, a surfactant.

The pharmaceutical composition may additionally or alternatively comprise one or more pharmaceutically acceptable diluents.

In an embodiment, the antibody or antibody conjugate of the pharmaceutical composition is lyophilised.

In an embodiment, the pharmaceutical composition further comprises one or more additional therapeutic agents, such as one or more additional drugs. In an embodiment, the pharmaceutical composition further comprises one or more chemotherapy drugs.

In an embodiment, the pharmaceutical composition is suitable for medical use in a human patient.

In an embodiment, the pharmaceutical composition is formulated for intravenous, subcutaneous, intramuscular, intrathecal, oral, sublingual, ocular, otic, or cutaneous administration. Preferably, the pharmaceutical composition is formulated for intravenous use. In an embodiment, the pharmaceutical composition is formulated for direct administration into a tumour (e.g. a brain tumour).

Methods of Treatment

The present invention further provides methods of treatment comprising therapeutic use of an antibody and/or antibody-conjugated nanoparticle of the invention.

In an embodiment, a method of treating or ameliorating the symptoms of a genetic disorder is provided, the method comprising administering to a patient a composition comprising an antibody conjugate, wherein said antibody conjugate comprises an antibody and a nanoparticle conjugated to the antibody, and said nanoparticle comprises a gene editing payload. An antibody conjugate comprising an antibody and a nanoparticle conjugated to the antibody for use in the treatment or amelioration of the symptoms of a genetic disorder, wherein the nanoparticle comprises a gene editing payload, is also provided.

In an embodiment, a method of treating or ameliorating the symptoms of a disease is provided, the method comprising administering to a patient a composition comprising an antibody conjugate, wherein said antibody conjugate comprises an anti-Gpr56 antibody and a nanoparticle conjugated to the antibody, and said nanoparticle comprises a therapeutic payload. An antibody conjugate comprising an anti-Gpr56 antibody and a nanoparticle conjugated to the antibody for use in the treatment or amelioration of the symptoms of a disease, wherein the nanoparticle comprises a therapeutic payload, is also provided.

In an embodiment, a method of treating or ameliorating the symptoms of a disease is provided, the method comprising administering to a patient a composition comprising an antibody conjugate, wherein said antibody conjugate comprises an anti-CD34 antibody and a nanoparticle conjugated to the antibody, and said nanoparticle comprises a therapeutic payload. An antibody conjugate comprising an anti-CD34 antibody and a nanoparticle conjugated to the antibody for use in the treatment or amelioration of the symptoms of a disease, wherein the nanoparticle comprises a therapeutic payload, is also provided.

The therapeutic payload is any payload as described herein, and is preferably a gene editing payload, optionally comprising a CRISPR nuclease. Most preferably, the payload is configured to edit the γ-globin gene locus in the patient's genome. Preferably, the γ-globin promoter is edited such that recruitment of BCL11A is inhibited. In this embodiment, the gene editing payload preferably comprises a CRISPR nuclease, such as Cas9, optionally SpCas9, and a gRNA having the sequence: CTT GTC AAG GCT ATT GGT CA (SEQ ID NO: 17) [9].

When the therapeutic payload is a gene editing disorder, the antibody conjugate is preferably used in a method of treating or ameliorating the symptoms of a genetic disorder. Preferably, the genetic disorder is a haematological disease, such as a haematological malignancy, or a hemoglobinopathy, for example sickle cell disease (SCD) or β-thalassemia.

In an embodiment, the method comprises administering the antibody conjugate intravenously, intrauterine, subcutaneously, intramuscularly, intrathecally, orally, sublingually, ocularly, oticly, or cutaneously. Preferably, the antibody conjugate is administered intravenously.

In an embodiment, the antibody conjugate for use in a method of treating or ameliorating a disease, such as a genetic disease, is provided in a pharmaceutical composition, such as any pharmaceutical composition described herein.

The methods disclosed herein permit in vivo treatment of a disease, such as a genetic disease, such as in vivo delivery of a gene editing payload, with minimised off-target effects.

In particular, the antibody molecule(s) to which the nanoparticle in conjugated target the nanoparticle payload to a target cell, and the payload is delivered exclusively or largely exclusively to the target cell. Preferably, the target cell is a hematopoietic stem cell. The methods disclosed herein may alternatively be used in ex vivo treatment of a disease, for examples in cell therapy.

In an embodiment, no more than 10% of the total payload administered to the patient via the antibody conjugated nanoparticles is delivered to non-target cells, such as no more than 5%, 4%, 3%, 2%, 1%, 0.5%, or 0.1%.

The invention also encompasses ex vivo methods for delivering a payload to target cells, such as for delivering a gene editing payload to target cells. In an embodiment, the method of treating or ameliorating the symptoms of a disease, such as a genetic disease, comprises removing autologous target cells from the patient, applying the antibody conjugated nanoparticles comprising a payload to the autologous target cells, confirming delivery of the payload to one or more target cells, wherein the payload is delivered when the payload is within the cell, and transplanting the one or more target cells comprising the delivered payload into the patient. In an embodiment, the autologous target cells are autologous stem cells, such as autologous hematopoietic stem cells. In an embodiment, the payload is a gene editing payload. In an embodiment, delivery of the payload is confirmed by observing a dye, such as a fluorescent dye, within the target cell. In this embodiment, the payload comprises a dye, such as a fluorescent dye, which may be conjugated to the therapeutic payload. In an embodiment, the fluorescent dye is DiD (1,1′-dioctadecyl-3,3,3′,3′-tetramethylindodicarbocyanine, 4-chlorobenzenesulfonate salt).

The invention further provides an ex vivo method for gene editing T cells comprising administering an antibody-conjugated nanoparticle comprising a gene editing payload to a population of cells comprising T cells (e.g. a population of peripheral blood lymphocytes) in vitro, wherein the antibody targets a T cell surface antigen (e.g. CD4, CD8, CD3, CTLA4, TCR, TCRα or TCRβ). In some embodiments, the gene editing payload is capable of knocking out PD-1. In some embodiments, the PD-1-knockout T cells are expanded ex vivo and then administered to a patient (e.g. a cancer patient, such as patient having oesophageal cancer, bladder cancer, prostate cancer, renal cell carcinoma or (non-small cell) lung cancer).

The invention further provides a method for treating cancer in a patient comprising:
(a) isolating a population of cells that comprises T cells from the patient, optionally wherein the population of cells is a population of peripheral blood lymphocytes,
(b) administering an antibody-conjugated nanoparticle comprising a gene editing payload to the population of cells in vitro, wherein the antibody targets a T cell antigen (e.g. CD4, CD8, CD3, TCR, TCRα or TCRβ), optionally wherein the gene editing payload is capable of knocking out PD-1, and
(c) administering the gene edited T cells to the patient.

The invention further provides an ex vivo method for gene editing hematopoietic stem cells (HSC) comprising administering an antibody-conjugated nanoparticle comprising a gene editing payload to a population of cells comprising HSC (e.g. in a blood sample or bone marrow extract) in vitro, wherein the antibody targets an HSC surface antigen (e.g. CD34, Gpr56, Gpr97, CD49, CD49f, CD90, CD117 or endomucin). In some embodiments, the gene editing payload is capable of repairing a genetic defect in the HSC. In some embodiments, the gene edited HSC are expanded ex vivo and then administered to a patient (e.g. a patient having a genetic disorder that results from the genetic defect, such as aplastic anaemia, sickle cell disease, thalassemia, haemoglobinopathy, or severe combined immunodeficiency).

The invention further provides a method for treating a genetic disorder in a patient comprising:
(a) isolating a population of cells that comprises HSC from the patient, optionally in a blood sample or bone marrow extract,
(b) administering an antibody-conjugated nanoparticle comprising a gene editing payload to the population of cells in vitro, wherein the antibody targets an HSC surface antigen (e.g. CD34, Gpr56, Gpr97, CD49, CD49f, CD90, CD117 or endomucin), optionally wherein the gene editing payload is capable of repairing a genetic defect that contributes to the genetic disorder,
(c) administering the gene edited HSC to the patient.

The invention further provides methods of treating or ameliorating the symptoms of a disease comprising administering to a patient an anti-CD34 antibody comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 1; an HCDR2 comprising the amino acid sequence of SEQ ID NO: 2; and an HCDR3 comprising the amino acid sequence of SEQ ID NO: 3; and the VL comprises an LCDR1 comprising the amino acid sequence of SEQ ID NO: 4; an LCDR2 comprising the amino acid sequence of SEQ ID NO: 5; and an LCDR3 comprising the amino acid sequence of SEQ ID NO: 6. Said anti-CD34 antibody for use in the treatment or amelioration of the symptoms of a disease is also provided.

The invention also provides methods for treating or ameliorating the symptoms of a disease comprising administering to a patient an anti-Gpr56 antibody comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3), wherein the VH comprises an HCDR1 comprising the amino acid sequence of SEQ ID NO: 9; an HCDR2 comprising the amino acid sequence of SEQ ID NO: 10; and an HCDR3 comprising the amino acid sequence of SEQ ID NO: 11; and the VL comprises an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14. Said anti-Gpr56 antibody for use in the treatment or amelioration of the symptoms of a disease is also provided.

In an embodiment, the anti-CD34 or anti-Gpr56 antibody is provided in a pharmaceutical composition, as described herein.

### EXAMPLES

The invention will now be described further by the following non-limiting examples.

### Example 1

The aim of this study was to provide a PLGA-PEG nanoparticle that could be specifically targeted to hematopoietic stem cells. This was achieved by conjugating the nanoparticles to novel anti-CD34/anti-Gpr56 antibodies.

Expression Pattern and Hematopoietic Potential of Gpr56+ and CD34+ Cells in Human Cord and Peripheral Blood

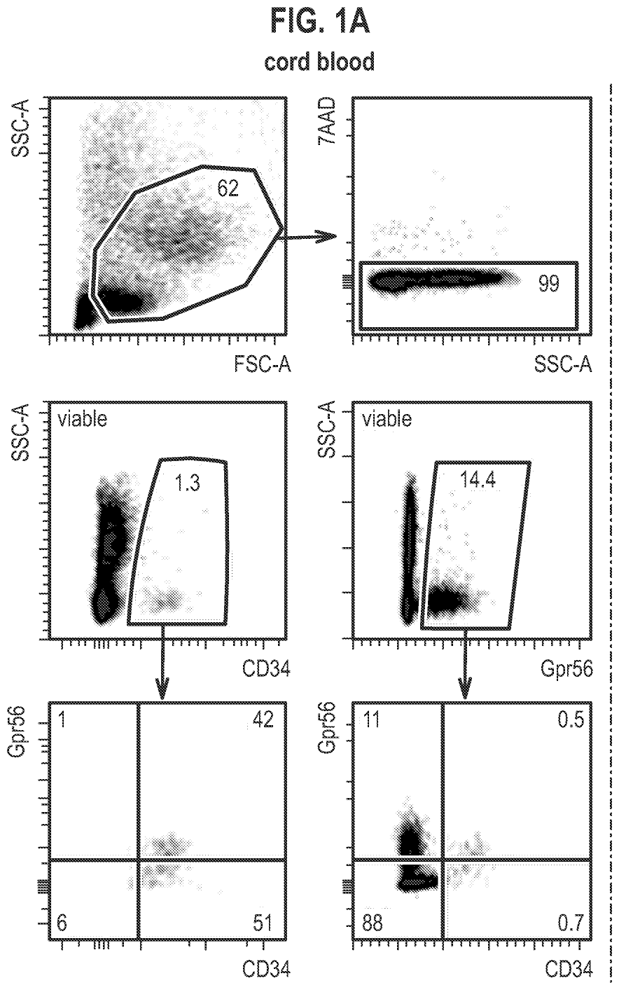

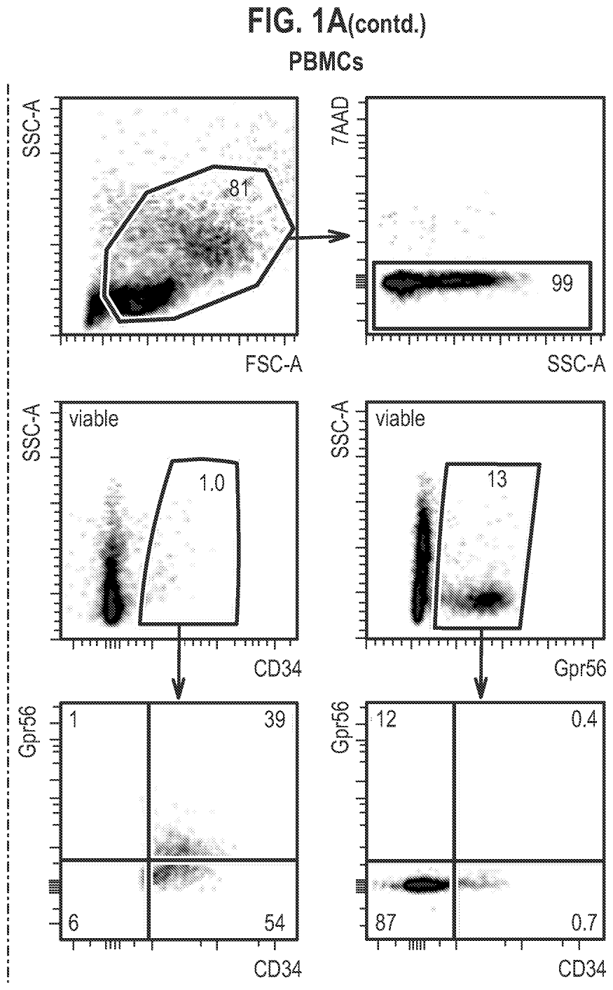

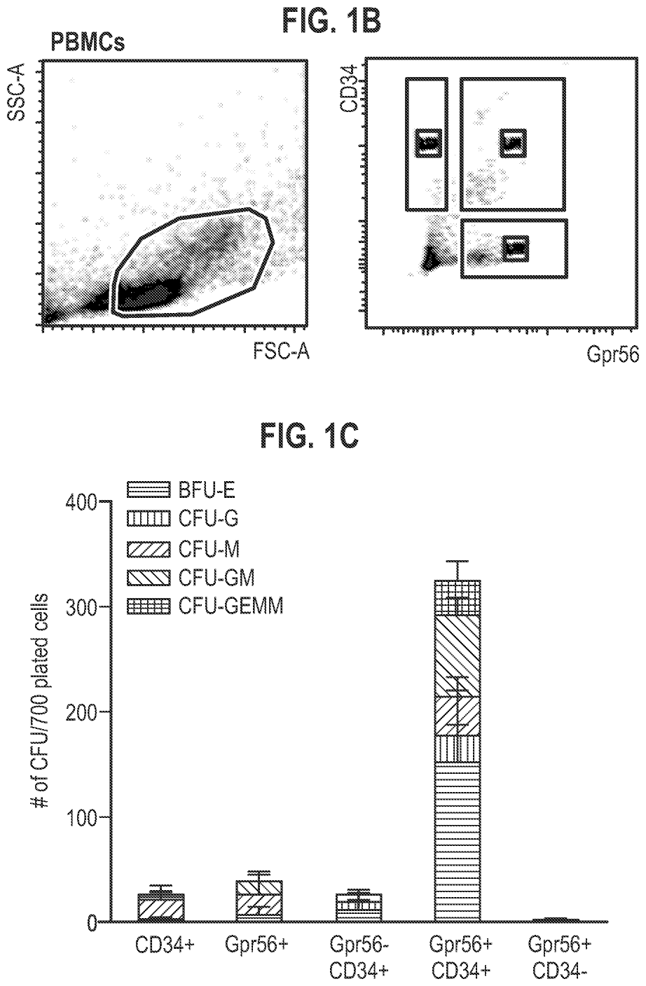

The expression patterns of CD34 and Gpr56 in human cord and peripheral blood was assessed by flow cytometry (FIG. 1A). While 1 to 1.3% of viable (7AAD−) cells were found to be positive for CD34 in peripheral and cord blood, respectively, Gpr56 showed a broader expression of 13 to 14.4%. Viable CD34 and Gpr56 cells were further analysed for concurrent expression of CD34 and Gpr56. The analysis showed that 42% of CD34 in cord blood and 39% in peripheral blood co-expressed CD34 and Gpr56. In contrast, only a small fraction of Gpr56+ cells co-expressed CD34, 0.5% in cord blood and 0.4% in peripheral blood.

To evaluate the hematopoietic potential of CD34+ and Gpr56+ expressing cells in peripheral blood, the Gpr56+, CD34+, CD34+Gpr56−, CD34+Gpr56+ and CD34−Gpr56+ populations were FACS-sorted (FIG. 1A-B), followed by methylcellulose culture, as described below. The number and type of hematopoietic progenitor colonies were counted (FIG. 1C). While no colonies were detected in the CD34− populations, a 10-fold increase was found in the number of hematopoietic colonies in the CD34+Gpr56+ population, compared to colonies derived from equal numbers of CD34+ and Gpr56+ positive cells.

In summary, while Gpr56 has a broader expression pattern than CD34, about 40% of the CD34+ population co-expressed Gpr56. Thus, the hematopoietic potential is highly enriched when combining the markers Gpr56 and CD34.

Generation of Novel, Fully Human α-Gpr56 and α-CD34 Antibodies

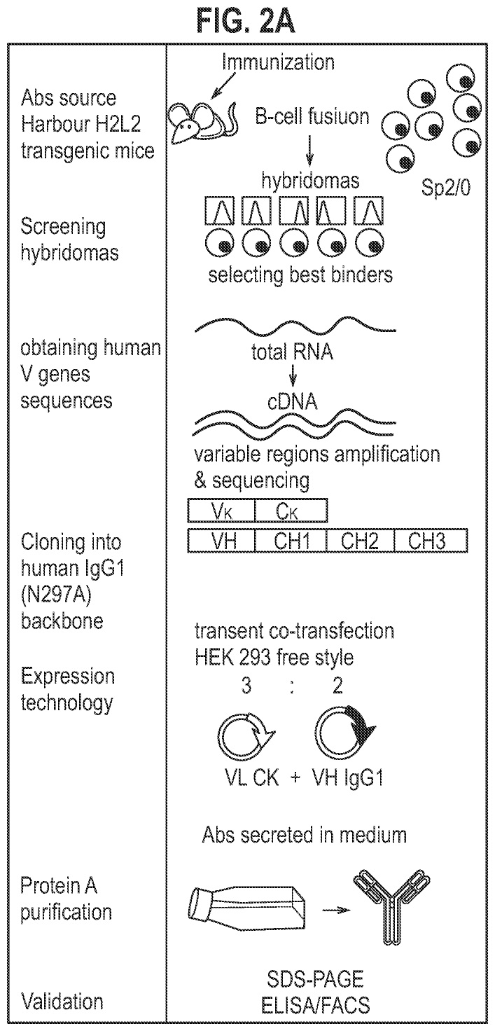

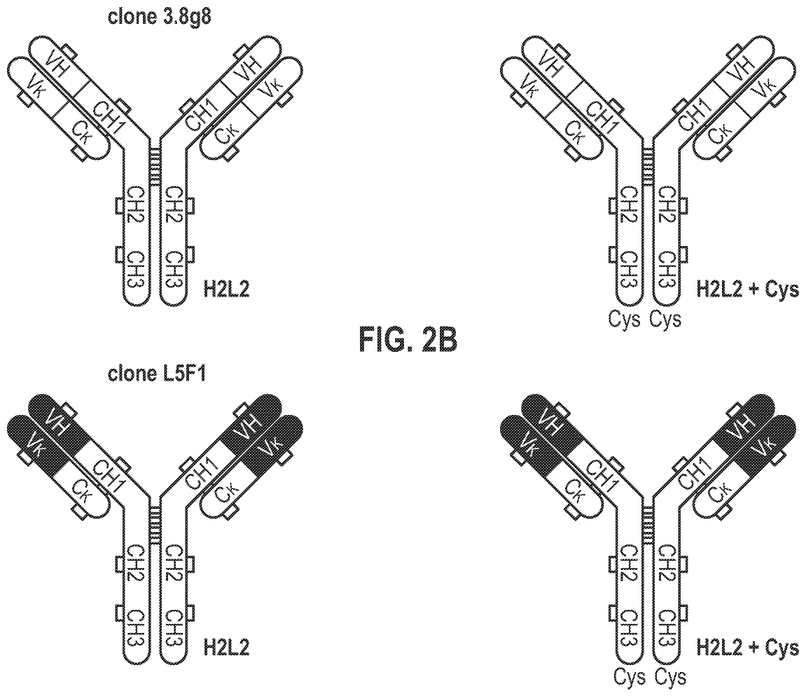

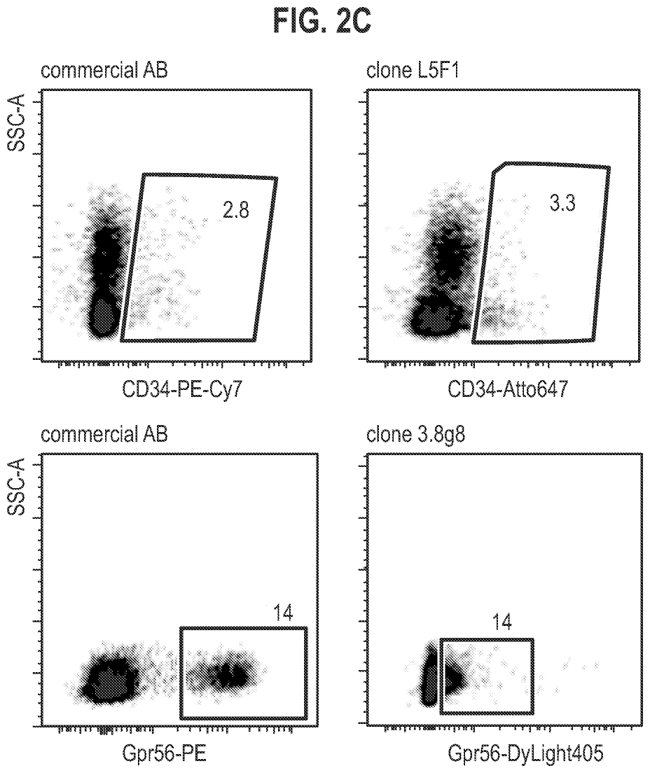

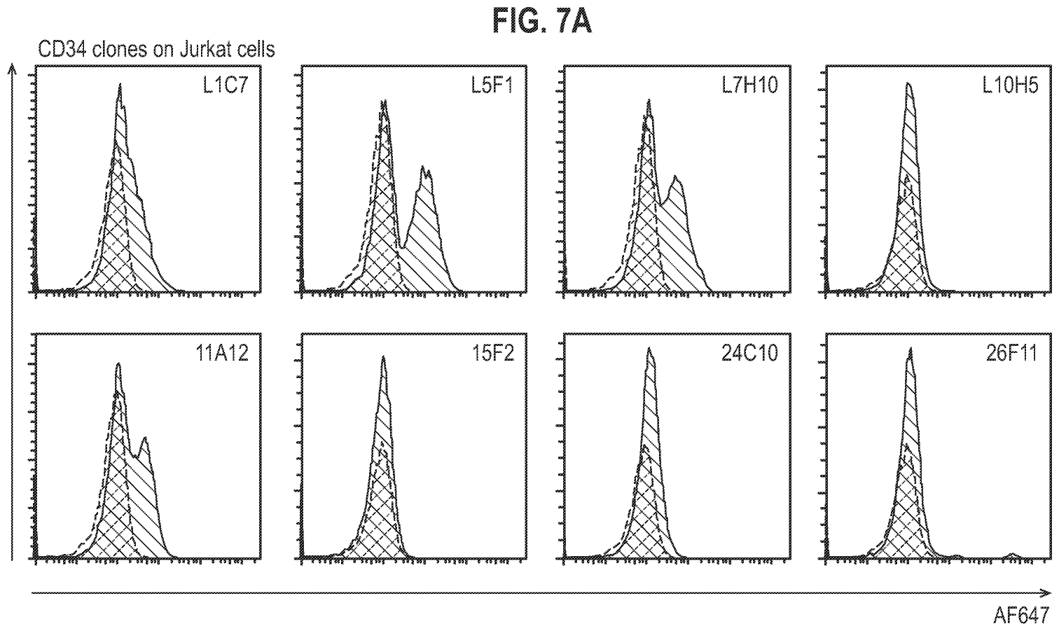

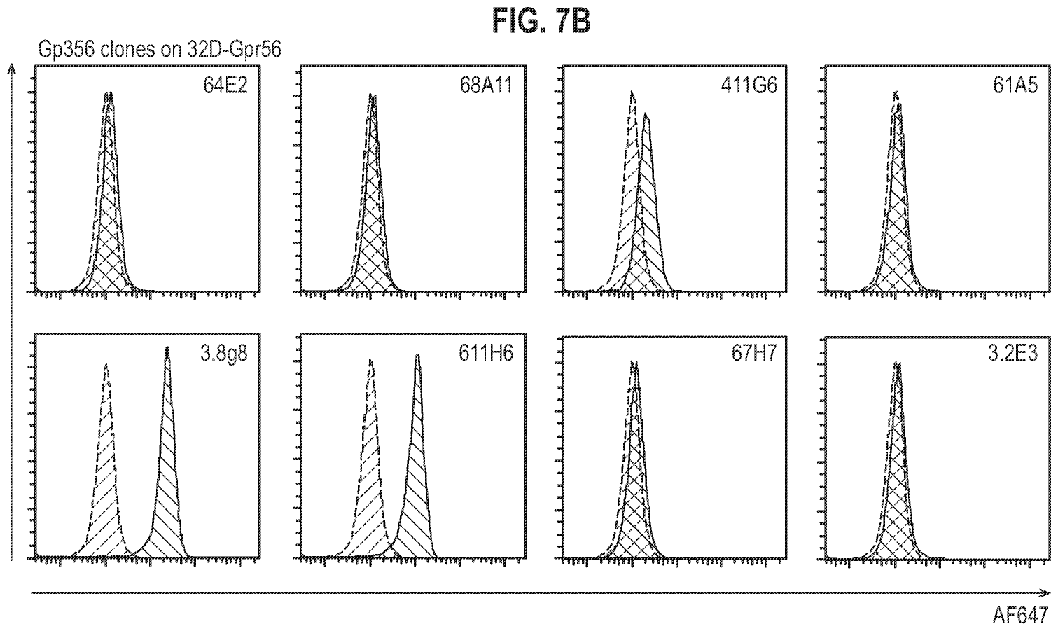

To target human HSCs for clinical application, fully human mAbs against the common and novel HSC markers CD34 and Gpr56 were generated, respectively, using the H2L2 transgenic mouse model (Harbour) that encodes chimeric immunoglobulins with human variable heavy and light chains and constant regions of rat origin. To this end, the peptide covering region 191 aa-207aa of extracellular domain of CD34 coupled to keyhole limpet haemocyanin (KLH) was used as an antigen for immunization of 6 H2L2 mice. For Gpr56, recombinant His-tagged soluble protein comprising the extracellular domain was used as the antigen for immunization of another group of 6 H2L2 mice (FIG. 2A). More than 5000 hybridomas were screened for each of the two independent fusion experiments by antigen-specific ELISAs. 15 CD34 peptide specific hybridomas were selected using the same peptide coupled to BSA for screening to exclude KLH positive hybridomas. All 15 CD34 positive hybridomas were sub-cloned, sequenced and produced for further characterisation. More than 70 hybridomas were Gpr56 positive. Supernatants were tested for binding affinities to Gpr56 expressing cell line (FIG. 7) and those binding with high affinities were sub-cloned, sequenced and produced for further characterisation. Hybridoma clone 3.8g8 directed against Gpr56, and clone L5F1 directed against CD34 showed best affinities towards primary cells and were chosen for recombinant fully human mAb production. The DNA encoding for variable regions of the heavy and light chains were cloned into expression plasmids containing the human IgG1 constant region and human kappa light chain constant region, respectively (Harbour BioMed vectors pHBM000267 and pHBM000265). The IgG1 backbone used carries an N297A mutation that reduces antibody-dependent cell-mediated cytotoxicity (ADCC), antibody-dependent cellular phagocytosis (ADCP), and complement-dependent cytotoxicity (CDC). A variant was made where an extra cysteine is added at the C terminal of the Fc-region (FIG. 2B). Recombinant human L5F1 and 3.8g8 mAbs and unspecific isotype control mAbs (anti-strep-tag and anti-MSLN) were produced in HEK-293 free style cells following transient co-transfection with two expression plasmids (encoding for appropriate combination of heavy and light chains). 7 days later, cells were spun down and human antibodies were purified from supernatants using Protein-A affinity chromatography. The recombinantly expressed human 3.8g8 and L5F1 were validated by flow cytometry. The recombinant human L5F1 and 3.8g8 mAbs showed similar expression pattern on cord blood as commercial Gpr56 and CD34 antibodies (FIG. 2C).

Formulation of Gpr56- and CD34-Targeting PLGA-PEG-NPs

To develop a carrier system that could be employed to specifically target and deliver a payload to HSCs, PLGA-PEG-NPs encapsulating the fluorescent dye DiD were formulated, using an o/w emulsion and solvent evaporation-extraction method (Table 1 below).

TABLE 1
Physicochemical characterization of PLGA-PEG-NPs

| | | | | |
|---|---|---|---|---|
| | | NP | | Zeta |
| Targeting | | diameter ± | | potential ± |
| moiety | NPs | SD (nm) | PDI | SD (nm) |

Conjugation via cysteine

| | | | | |
|---|---|---|---|---|
| None | NP-(DiD)- | 311.85 ± | 0.2 ± | −12.3667 ± |
| | PEG2000- | 2.97 | 0.02 | 0.677249 |
| | maleimide | | | |
| | functionalized | | | |
| α-Gpr56- | NP-(DiD)- | 336.73 ± | 0.2 ± | −16.3 ± |
| Cys | maleimide | 1.24 | 0.01 | 0.173205 |
| | functionalized | | | |
| α-CD34- | NP-(DiD)- | 354.55 ± | 0.19 ± | −17.333 ± |
| Cys | maleimide | 21.23 | 0.03 | 0.557375 |
| | functionalized | | | |
| α- | NP-(DiD)- | 423.07 ± | 0.35 ± | −17.03 ± |
| mesothelin- | maleimide | 27.59 | 0.08 | 1.091177 |
| Cys | functionalized | | | |

Conjugation via amino group (lysines)

| | | | | |
|---|---|---|---|---|
| None | NP-(DiD)- | 393.40 ± | 0.39 ± | −14.8 ± |
| | PEG-NHS- | 18.03 | 0.08 | 0.360555 |
| | ester | | | |
| α-Gpr56- | NP-(DiD) | 453.60 ± | 0.43 ± | −8.18 ± |
| Cys | | 46.8 | 0.24 | 0.16095 |
| α-CD34- | NP-(DiD) | 434.14 ± | 0.40 ± | −10.8217 ± |
| Cys | | 73.2 | 0.33 | 0.6648483 |
| α- | NP-(DiD) | 475.70 ± | 0.37 ± | −15.033 ± |
| mesothelin- | | 70.70 | 0.05 | 0.725718 |
| Cys | | | | |

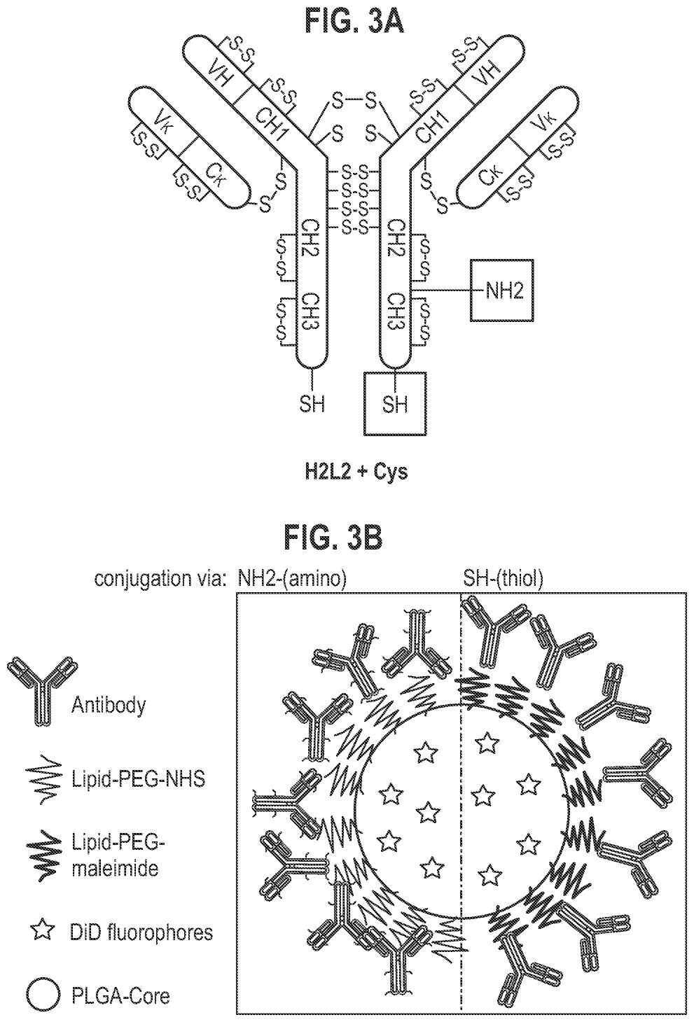

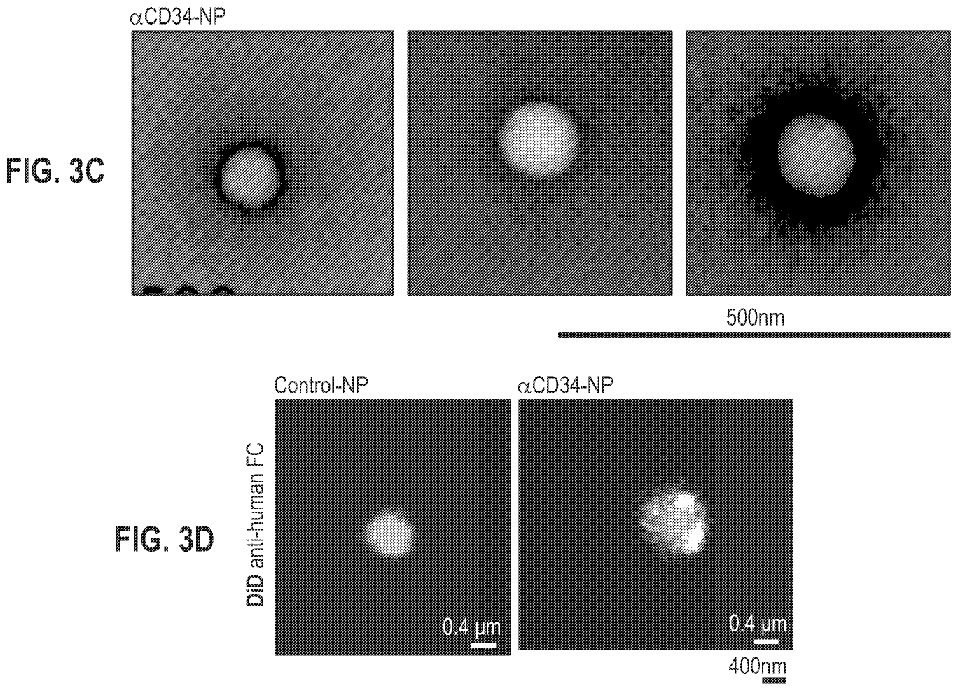

To provide specificity for the targeting of HSCs, recombinant human L5F1 and 3.8g8 mAbs was conjugated to the surface of PLGA-NPs using two different conjugation strategies (FIG. 3A). In strategy 1, primary amines present in lysine residues of the mAbs were used. Lysines are abundant, widely distributed and easily modified because of their reactivity and their location on the surface of the mAb. In strategy 2, the mAbs were conjugated via cysteine residues (FIG. 3A). Cysteines are less abundant than lysines and these cysteines exclusively form covalent disulfide bonds to stabilize the tertiary structure of the mAb and are therefore, under non-reducing conditions, not reactive. By cloning in a cysteine residue on to the C-terminus of the heavy chains an additional unpaired cysteine residue was obtained, harbouring a free thiol group at the heavy chain Fc region for site-specific conjugation to the NPs.

The PLGA-NPs were functionalized with lipid-PEG(2000)-NHS to conjugate the mAbs via a N-hydroxysuccinimide (NHS) ester-mediated reaction (strategy 1) or PLGA-NPs were functionalized with lipid-PEG(2000)-maleimide to conjugate the mAb via a specific reaction of the maleimide-group (PEG-maleimide) with the thiol (sulfhydryl group) (strategy 2) (FIG. 3B). While in strategy 1, any lysine residue could be employed for conjugation resulting in a random orientation of the mAb on the PLGA-NP-surface, strategy 2 allowed site-specific orientation of the mAb with the binding grooves facing away from the NP-core and allowing optimal engagement with targeted molecules.

The size of the PLGA-NPs was determined by DLS analysis as 311-475 nm in diameter (Table 1) and the polydispersity index (PDI) values of 0.2-0.4 obtained from the DLS measurement indicated a homogeneous size distribution (Table 1). In addition, ZetaSizer measurement showed a negative surface charge of the NPs (Table 1). In contrast to control NPs without a targeting moiety, conjugation of mAbs slightly increased the size of the NPs (Table 1). TEM analysis confirmed a round of shape of the NPs (FIG. 3C). Confocal analysis of CD34-targeting PLGA-PEG-NPs confirmed the presence of recombinant human L5F1 mAb on the NP-surface (FIG. 3D).

Binding of Gpr56- and CD34-Targeting PLGA-PEG-NPs in Peripheral Blood

To evaluate the specificity of targeting PLGA-PEG-NPs towards CD34+ HSPCs within peripheral blood, and whether the conjugation strategy (and orientation) of the mAb to the NP-surface would influence the binding efficacy, PBMCs from healthy blood cell donors were incubated for 15 minutes at 37° C. with PLGA-PEG-NPs conjugated to anti-MSLN and anti-strep-tag control mAbs, as well as the HSC targeting mAbs anti-Gpr56 and anti-CD34 (all incorporating an additional unpaired cysteine at the Fc tail). Each mAb was conjugated via NHS-ester and maleimide coupling strategies (Table 1). The NPs co-encapsulated the fluorescent dye DiD.

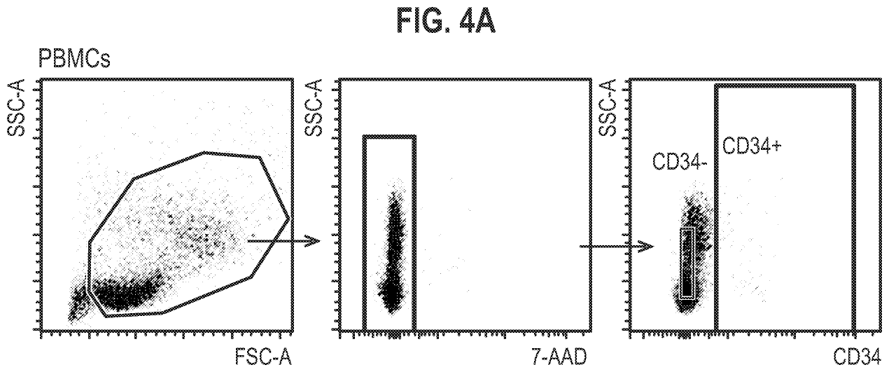

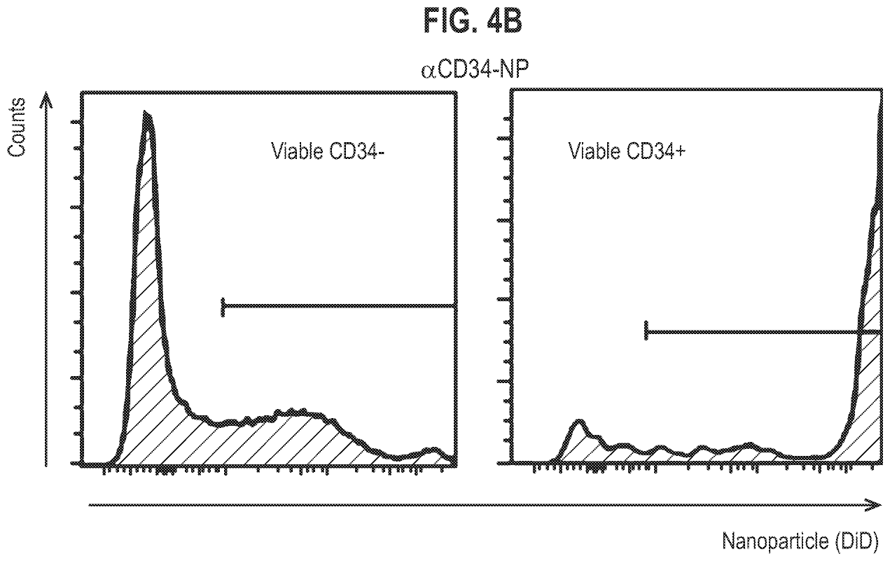

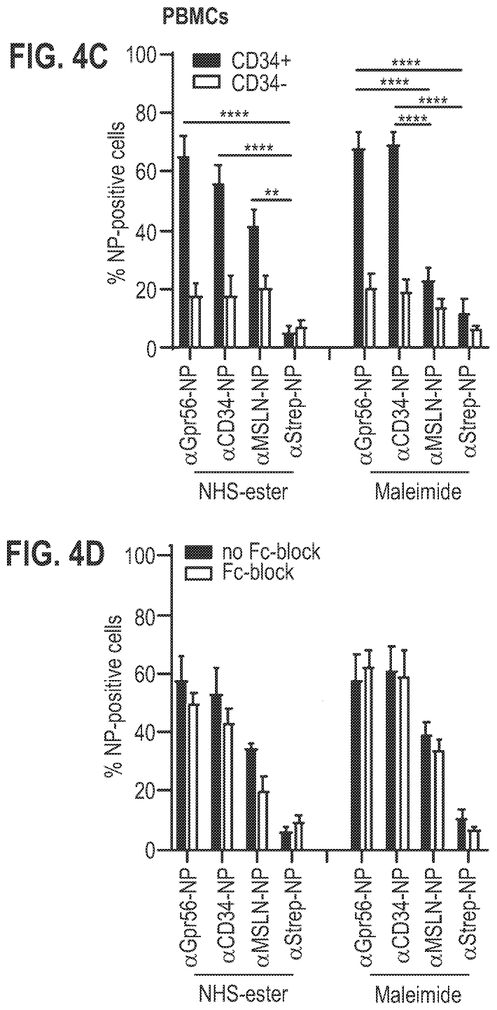

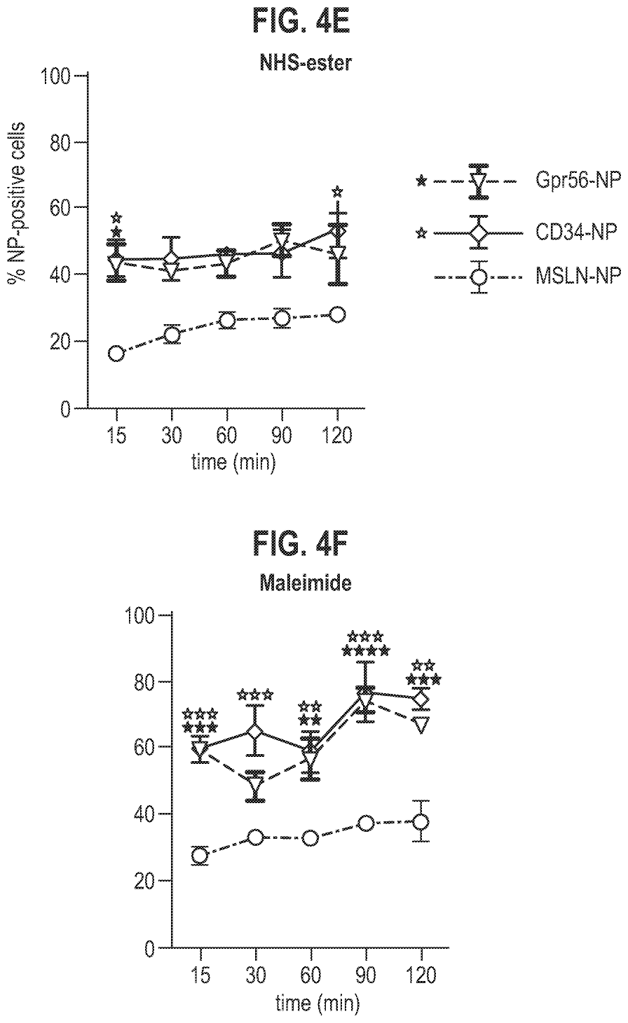

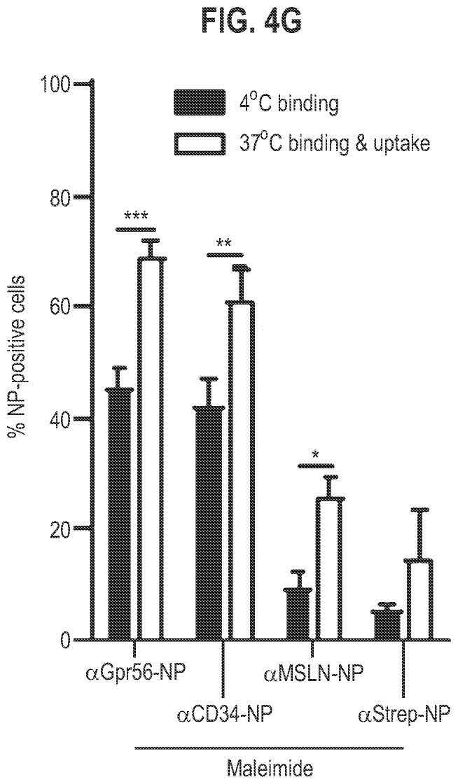

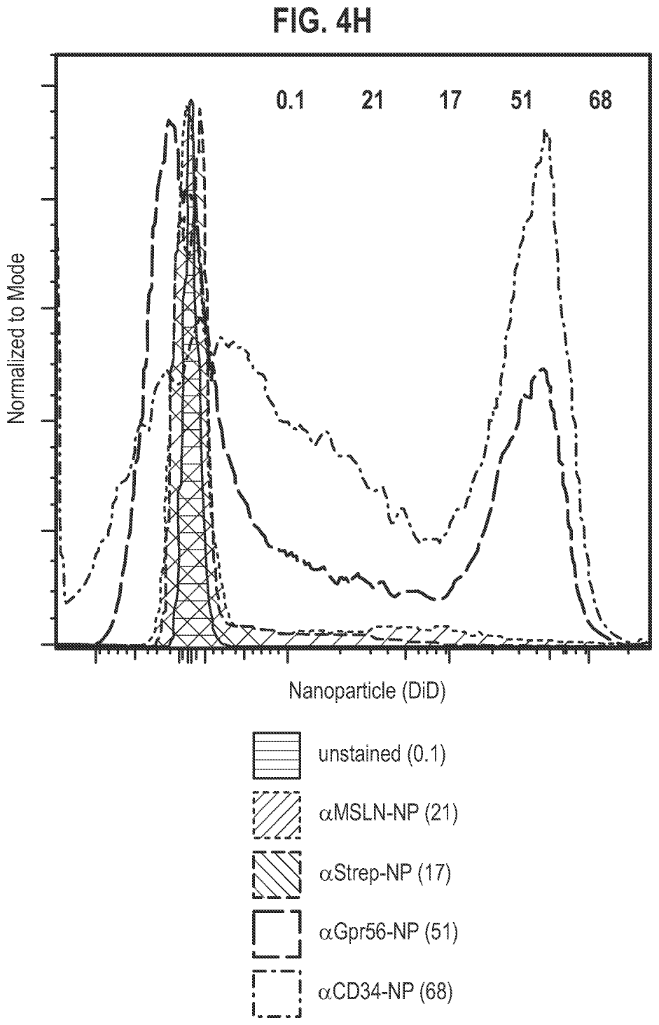

CD34 is commonly used to mark HSPCs in blood and bone marrow. Thus, after NP-incubation, PBMCs were intensively washed and labelled for CD34 and the viability dye 7AAD. NP-binding was determined by measuring the percentage of DiD-positive cells within the viable CD34+ and CD34− cell populations (FIG. 4A-B). CD34+ cells were efficiently targeted by anti-CD34-PLGA-PEG-NPs, and only residual binding was observed in CD34-cells (FIG. 4B).

Extensive analysis including multiple blood cell donors showed for both conjugation strategies efficient targeting of CD34+ HSPCs using Gpr56- and CD34-PLGA-PEG-NPs (FIG. 4C), while 10-20% targeting was observed in the CD34− population with all mAbs (control and targeting), similar to the percentages obtained by the control mAbs. The percentage of binding was slightly increased using the maleimide conjugation strategy, suggesting that the outwards pointing orientation of the mAb binding groove increased the binding of antibody-conjugated PLGA-PEG-NPs to CD34+ PBMCs.

As Fc-receptors are abundant in human blood cells, the potential contribution of Fc receptors to NP-binding was evaluated. To this end, binding studies were performed in the absence and presence of an Fc-receptor blocking reagent (FIG. 4D). Pre-treatment with an Fc-receptor blocking reagent did not decrease the binding of targeting and control PLGA-PEG-NPs, irrespective of the conjugation strategy, suggesting that unspecific Fc-receptor-mediated binding does not play role in the binding of CD34− and gpr56-PLGA-PEG-NPs. Thus, the basal binding of 10-20% that was observed in CD34− cells and control-PLGA-PEG-NPs is likely the result of unspecific pinocytosis.

Next, the uptake kinetics of NPs coated with α-Gpr56, α-CD34, α-MSLN or α-Strep mAbs, conjugated by NHS-ester or maleimide-thiol reaction was determined (FIG. 4E-F). PBMCs from 1 donor were incubated with PLGA-PEG-NPs for up to 120 minutes. The percentage of CD34+ cells that had internalized the fluorescent carriers was determined by flow cytometry. After 15 minutes, the targeting PLGA-PEG-NPs conjugated to mAbs via maleimide-thiol reaction bound to significantly more CD34+ PBMCs (FIG. 4F) than the NPs conjugated to mAbs via NHS-ester reaction (FIG. 4E). The uptake increased from 40-50% and 60-80% in NHS-ester conjugated and maleimide-conjugated NPs, respectively. Binding of NPs conjugated to mAbs via maleimide reaction was at all timepoints significantly higher than the control-NPs conjugated to α-MSLN mAb (FIG. 4E-F).

To dissect the binding and uptake behaviour of CD34+ cells, additional flow cytometry studies were performed. To this end, CD34+ cells were incubated for 1 hour with PLGA-PEG-NPs at 4° C., when cells can bind but not take up NPs, and at physiological conditions at 37° C. (FIG. 4G). After 1 hour, 40% of targeting PLGA-PEG-NPs bound to the surface of CD34+ PBMCs, in contrast to <10% in control-NPs. At physiological conditions, these values increased to >60-70% in targeting PLGA-PEG-NPs and ˜20% in control NPs. Thus, after 1 hour at physiological conditions, 20-30% (values obtained at 37° C.—values obtained at 4° C.) of targeting PLGA-PEG-NPs were taken up by CD34+ cells.

PBMCs represent a mixed cell population, with 1-2% CD34+ cells. Next, the binding of PLGA-PEG-NPs conjugated to α-Gpr56, α-CD34 and α-MSLN conjugated by maleimide-thiol reaction to isolated CD34+ blood cells from PBMCs was assessed. CD34+ cells were isolated from PBMCs with the help of CD34+ magnetic beads, and subsequently incubated with 10 μg/ml PLGA-PEG-NPs. 51% and 68% of isolated CD34+ cells bound PLGA-PEG-NPs coated with α-Gpr56 or α-CD34 mAb, respectively, while 17% bound α-MSLN and 21% α-Strep coated PLGA-PEG-NPs (FIG. 4H).

In summary, the data showed that CD34+ cells within PBMCs and isolated CD34+ cells specifically bound PLGA-PEG-NPs coated with targeting α-Gpr56 or α-CD34 mAbs over control mAb-coated PLGA-PEG-NPs. The uptake of targeting PLGA-PEG-NPs was slightly increased when the antibodies were conjugated via maleimide-reaction using the introduced free thiol group at the Fc region.

Uptake of α-Gpr56- and α-CD34-PLGA-PEG-NP by HSPCs Visualized by Confocal Microscopy

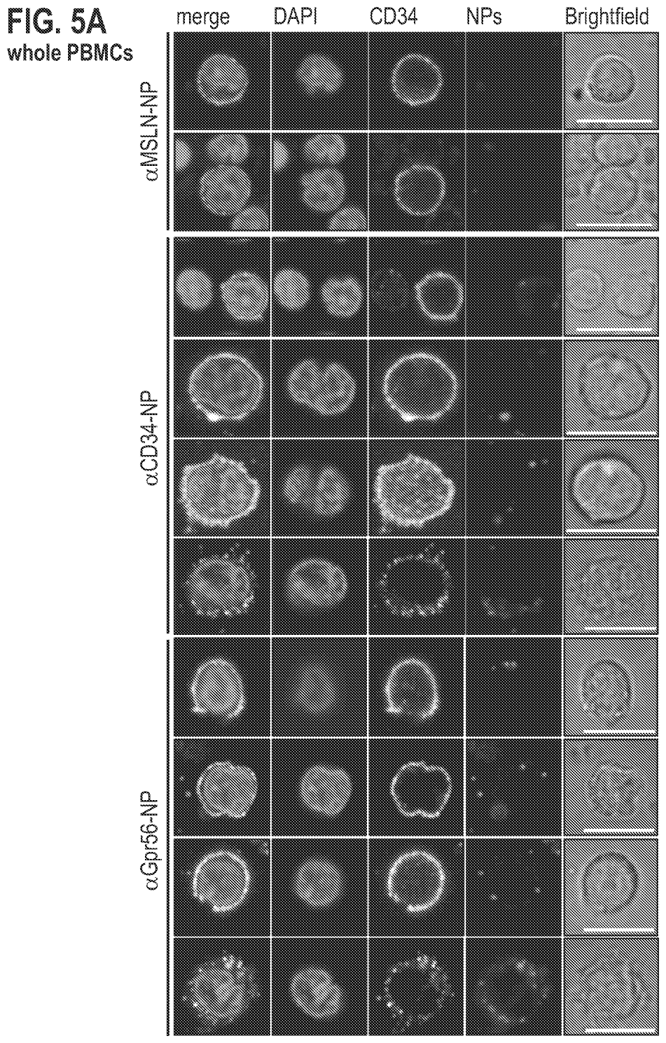

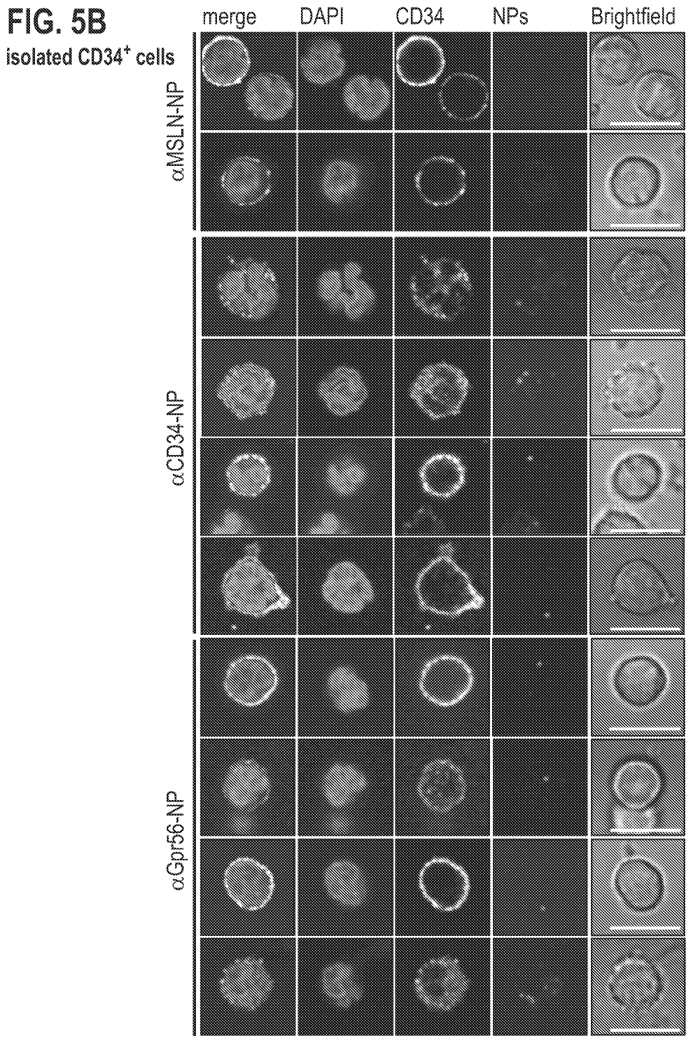

Long-term repopulating HSCs are quiescent cells and their metabolism differs from other HSPC populations. These small cells with little cytoplasm represent only a small fraction of all CD34+ cells. A prerequisite for an HSC-delivery system is efficient uptake by these cells. To confirm the intracellular localization in HSCs, whole PBMCs (FIG. 5A) and isolated CD34+ cells (FIG. 5B) were incubated for 1 hour with NPs coated with α-gpr56, α-CD34 and α-MSLN (conjugated via maleimide-thiol) and analysed by confocal microscopy.

Targeting α-Gpr56- and α-CD34-PLGA-PEG-NPs bound and were readily taken up by CD34low expressing cells that showed the typical nuclei structure of phagocytes. Some unspecific uptake of control α-MSLN-PLGA-PEG-NPs in these cells was also found (FIG. 8). Importantly, targeting α-Gpr56- and α-CD34-PLGA-PEG-NPs were found to be specifically bound and taken up by small and round CD34high expressing cells (HSCs) within PBMCs and isolated CD34+ cells, but not by control α-MSLN-PLGA-PEG-NPs (FIG. 5A-B).

Uptake of α-Gpr56-PLGA-PEG-NP and α-CD34-PLGA-PEG-NP in Distinct HSPC Populations within Peripheral Blood

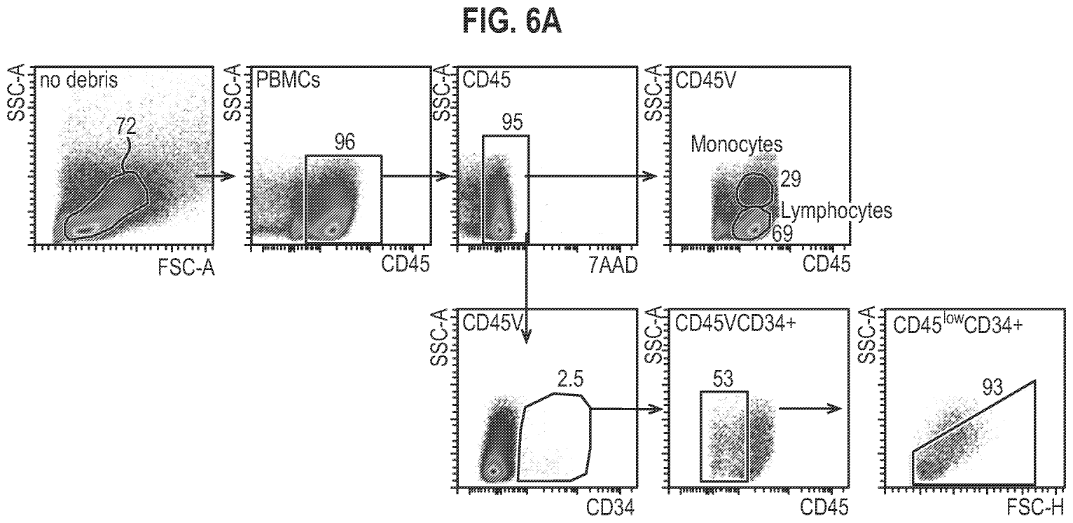

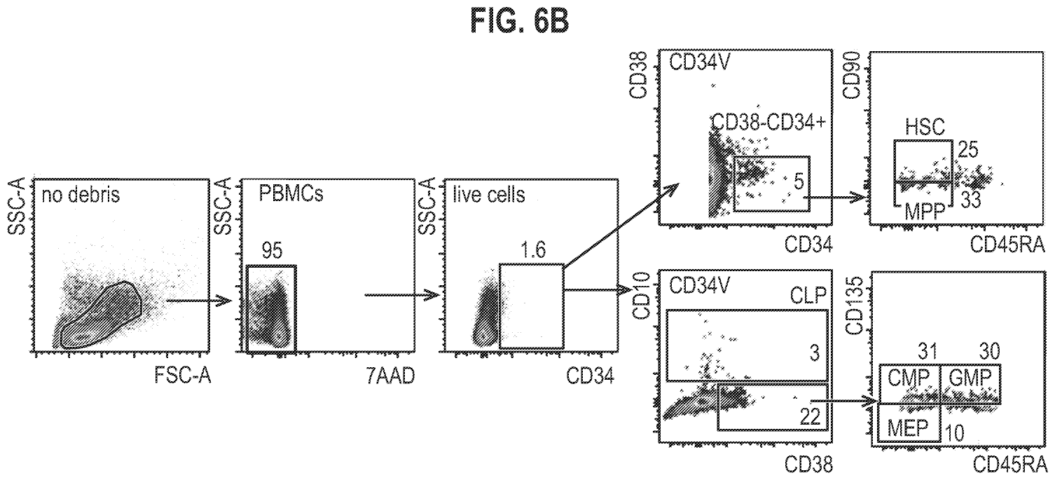

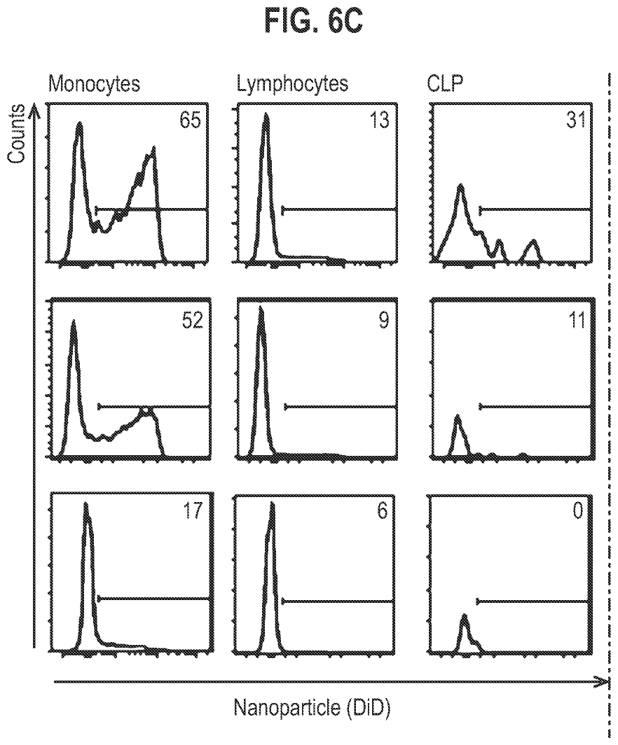

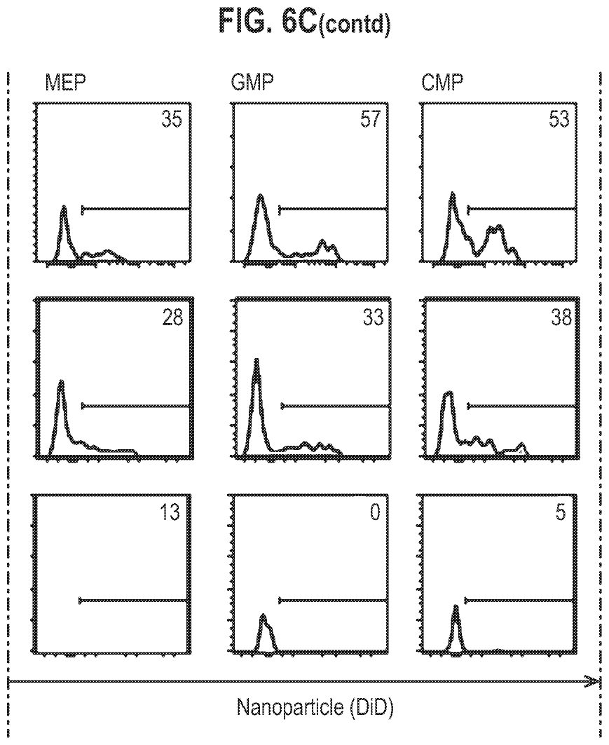

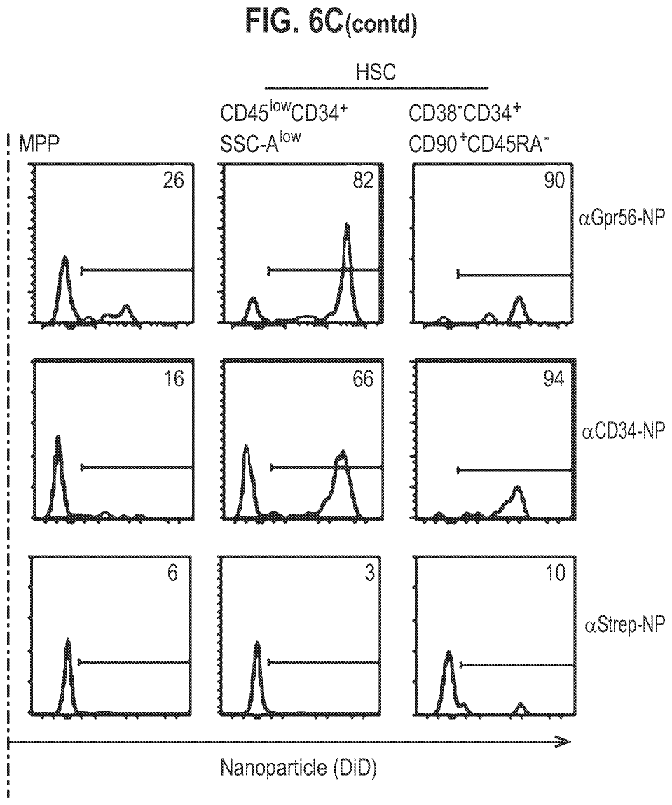

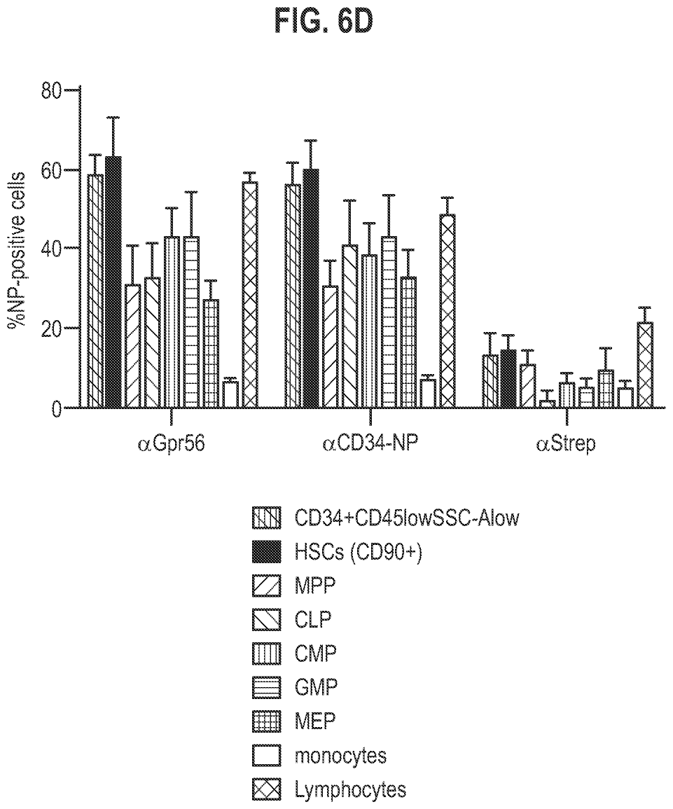

The HSPC populations within the CD34+ pool of PBMCs can be distinguished by flow cytometry by hematopoietic marker expression. In addition, according to the hematopoietic lineage potential, corresponding metabolism and differentiation status, they possess distinct uptake capacities. To specifically deliver therapeutics to HSPC populations, it is important to understand the differential uptake behaviour towards targeting α-Gpr56- and α-CD34-PLGA-PEG-NPs. To this end, =a 9-colour flow cytometry panel and a sequential Boolean gating strategy was employed following the Clinical and Laboratory Standards Institute (CLSI), which allowed analysis of the uptake of targeting and control-PLGA-PEG-NPs within 9 HSPC populations. PBMCs were incubated for 15 minutes at 37° C. with 20 μg/ml α-Gpr56-, α-CD34− and α-Strep-PLGA-PEG-NP and subsequently stained for flow cytometry. Dead cells were excluded by 7-AAD-positive staining. Monocytes and lymphocytes could be gated based on their morphology within the CD45V population (FIG. 6A). The HSC populations could be narrowed down by expression of CD34+CD45low and based on their size/granularity.

Viable CD34+ cells (CD34V) were further gated into CD34+CD38−, CD10+CD38− (common lymphoid progenitors, CLP) and CD10−CD38+ populations. The CD34+CD38− events were then subdivided further into a multipotent progenitor cell (MPP) and a true HSC population, defined as CD34+CD38−CD90−CD45RA− and CD34+CD38−CD90+CD45RA−, respectively. CD10−CD38+ cells could be further divided into CD135+CD45RA− (common myeloid progenitors, CMP), CD135+CD45RA+(granulocyte macrophage progenitor cells, GMP) and CD135−CD45RA− (megakaryocyte erythroid progenitor cells, MEP). Within each HSPC population, the percentage of NP (DiD)-positive cells was analysed (FIG. 6B).

Analysis of the HSPC populations showed efficient targeting (˜60%) of HSCs using α-Gpr56- and α-CD34-PLGA-PEG-NPs, and residual binding of control α-Strep-PLGA-PEG-NPs (˜10-20%). All other HSPC populations were targeted by ˜40% using both α-Gpr56- and α-CD34-PLGA-PEG-NPs. It is well known that monocytes efficiently take up NPs. Accordingly, 60% of monocytes were found to have taken up α-Gpr56- and α-CD34-PLGA-PEG-NPs. In contrast, lymphocytes, which express very low levels of CD34 and are non-phagocytic, were not efficiently targeted.

The materials and methods used in this study are described in further detail below.

Materials

For the preparation of nanoparticles (NPs), dichloromethane (DCM) and dimethylformamide (DMF) were purchased from Sigma-Aldrich® (Zwijndrecht, The Netherlands). Poly (D, L-lactic-co-glycolic acid) 50:50 (PLA/PGA) and polyvinyl alcohol (PVA) were purchased from Sigma-Aldrich. DiD was purchased from ThermoFisher (Waltham, Massachusetts, USA). PEG-DSPE-200 and PEG-DSPE-maleimide were purchased from Sigma Aldrich.

For Methylcellulose assays, MethoCult™ H4434 Classic medium was purchased from STEMCELL Technologies (Vancouver, Canada).

For flow cytometry, cells were stained with anti-CD10-FITC (clone HI10a), anti-CD135-PE (clone BV10A4H2), anti-CD45RA-APC-Cy7 (clone H1100), anti-gpr56-PE (clone CG4) (all from BioLegend, California, US), anti-CD34-PE-Cy7 (clone 581), CD90 OptiBuild BV711 (clone 5E10), anti-CD38 BD Horizon V450 (clone HIT2), anti CD45 BD Horizon V500 (clone H130) (all from BD biosciences) and 7-AAD (Invitrogen). Fc-receptors were blocked with anti-human FcR Blocking Reagent (Miltenyi Biotec, Bergisch Gladbach, Germany).

For microscopy, cells were stained with anti-CD34 antibody (clone 561, BioLegend) and detected with the secondary goat anti-mouse IgG (H+L)-Alexa Fluor 488 antibody (Invitrogen, ThermoFisher Scientific).

Antibody Generation

Production of mAbs 3.8g8 (α-Gpr56), L5F1 (α-CD34), 1.2e8(α-Mesothelin (MSLN) and 1.15f2 anti-strep-tag monoclonal antibodies (mAb):

6 H2L2 transgenic mice were immunised with the CD34 specific peptide coupled with keyhole limpet haemocyanin (LEQNKTSSCAEFKKDRG [SEQ ID NO: 18]) by Genscript. Human Gpr56 extracellular domains (ECD) were cloned into a pCAG hygro G2 vector after removing the G2 constant region, expressed in HEK 293 cells as his-tagged fusion proteins, purified on Ni-NTA agarose beads and used for immunization of the 6 H2L2 transgenic mice. Antigens were injected at 20 μg/mouse using Stimune Adjuvant (Prionics) freshly prepared according to the manufacturer's instruction for first injection, whereas boosting was done using Ribi (Sigma) adjuvant. Injections were done subcutaneously in two-week intervals and the last boost was intraperitoneal. Sera were tested after 3rd and 6th injections in an antigen specific ELISA. Four days after the last intraperitoneal injection, spleen and lymph nodes were harvested and hybridomas made by standard method using SP 2/0 myeloma cell line (ATCC #CRL-1581) as a fusion partner. More than 10000 hybridomas in total for both experiments (CD34 and Gpr56) were screened in antigen-specific ELISAs.

Antigen Specific ELISA on Serum and Hybridoma Supernatants

ELISA was carried out in 96 well plates (Nunc™ MaxiSorpR). 5 μg/ml antigen in PBS was used for coating for 2 hours at room temperature (RT). Antigen used for CD34 was the same peptide used for immunization but coupled to BSA, while for the Gpr56 the antigen was the same protein used for immunization. After blocking with 300 μl of PBS/1% BSA/1% fat free milk powder and 0.1% Tween-20 per well for 30 minutes at room temperature, plates were washed 3 times with the washing buffer (PBS/0.1% Tween-20). In case of testing titres of antibodies in sera, 3 μl of serum was diluted in 600 μl in PBS/1% BSA/1% fat free milk powder/0.1% Tween-20 followed by 1:1000, 1:3000, 1:7500, 1:15000, 1:30000, 1:60000 and 1:120000 dilution.

50 μl of each serum dilution in case of blood ELISA or 50 μl of hybridoma supernatant were added to the wells and incubated for at least 2 hours at RT or overnight at 4° C.

After washing the plates in washing buffer 5 times, 50 μl of horseradish peroxidase-labelled mouse anti-rat IgG antibody (Absea) diluted in blocking solution 1:2000 each was added (clone KT148 anti IgG1, clone KT98 anti IgG2b and clone KT99 anti IgG2c were mixed together). After 2 hours incubation at RT, the plates were washed 5 times with washing buffer, and 50 μl of POD substrate (Roche, BM Blue POD substrate soluble, #11484281001) was added. After 3-5 minutes the reaction was stopped by addition of 50 μl of 1M H₂SO₄ and the absorption was measured at 450 nm (against a reference wavelength of 690 nm). For all positive hybridomas, another ELISA assay was performed to determine the isotype, using each of the mouse anti-rat IgG secondary antibodies separately.

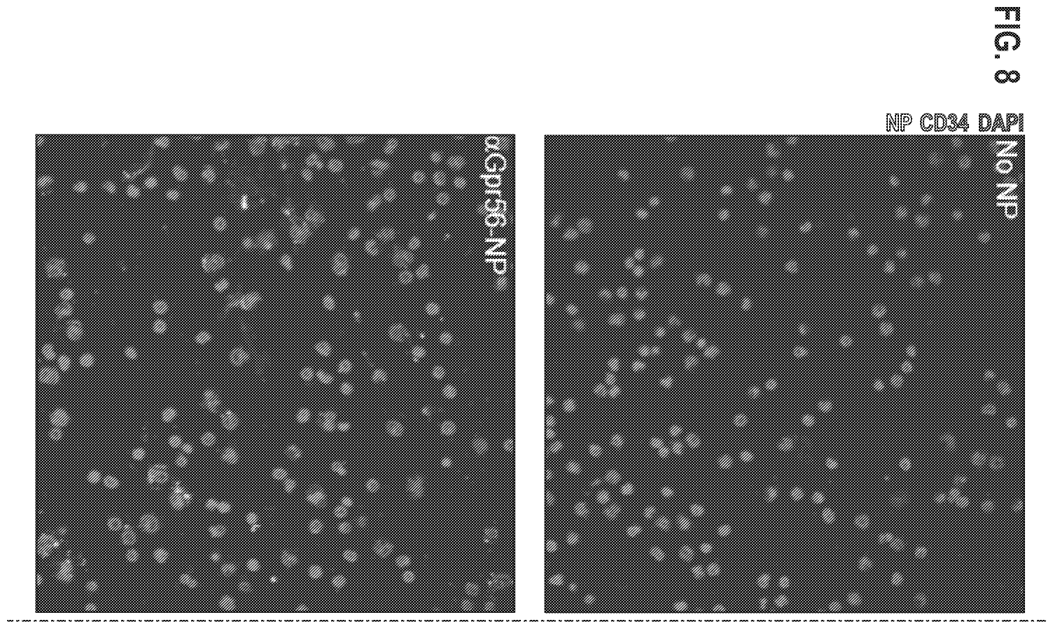

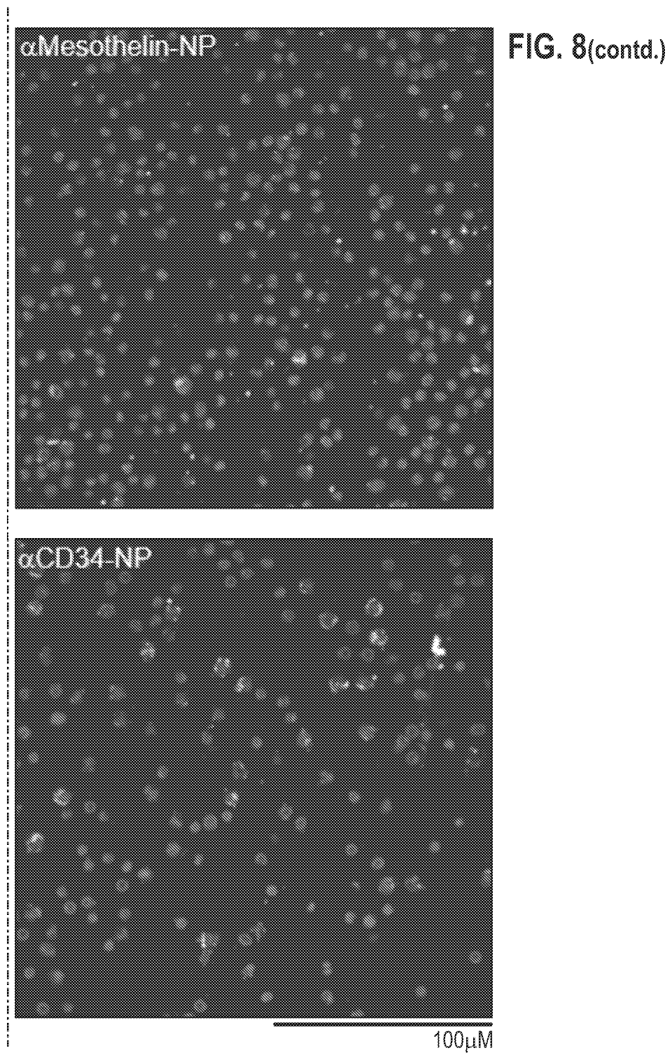

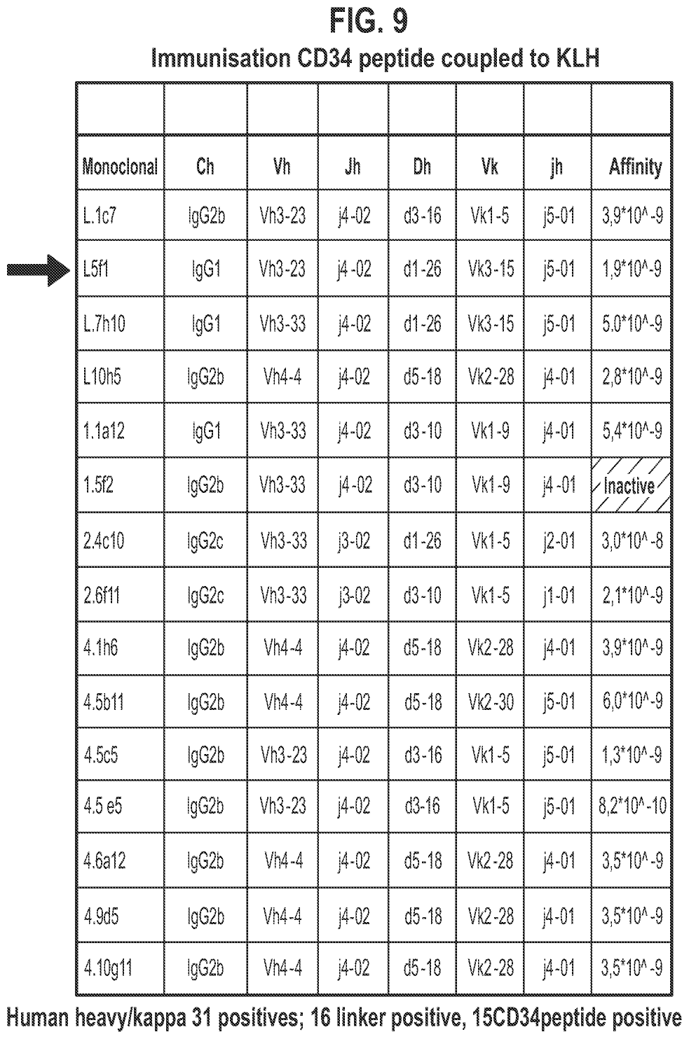

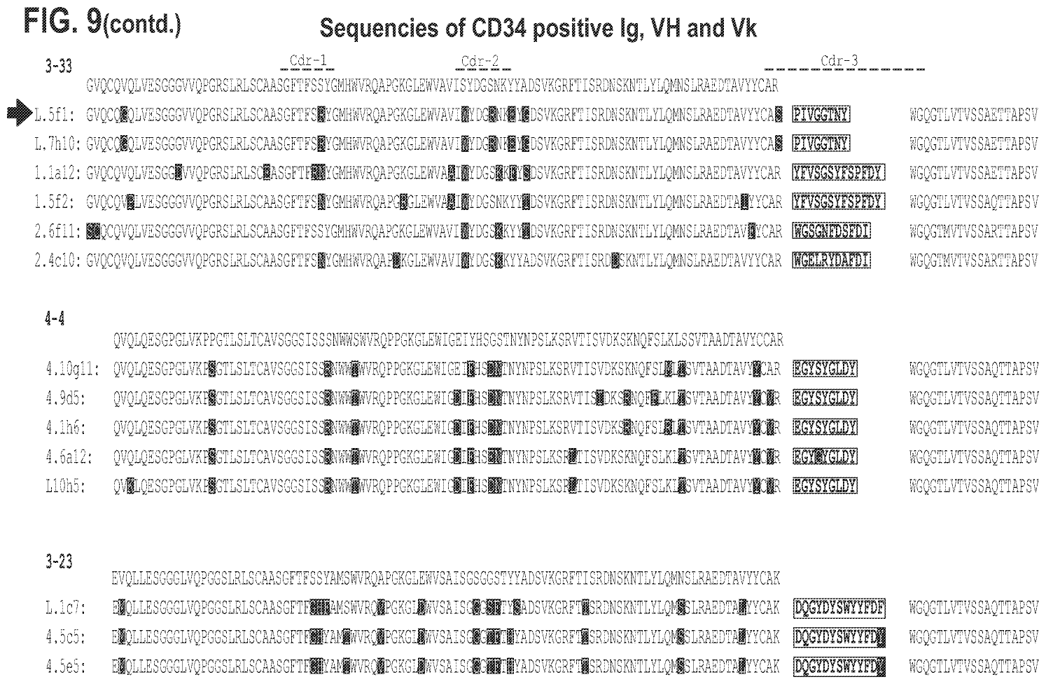

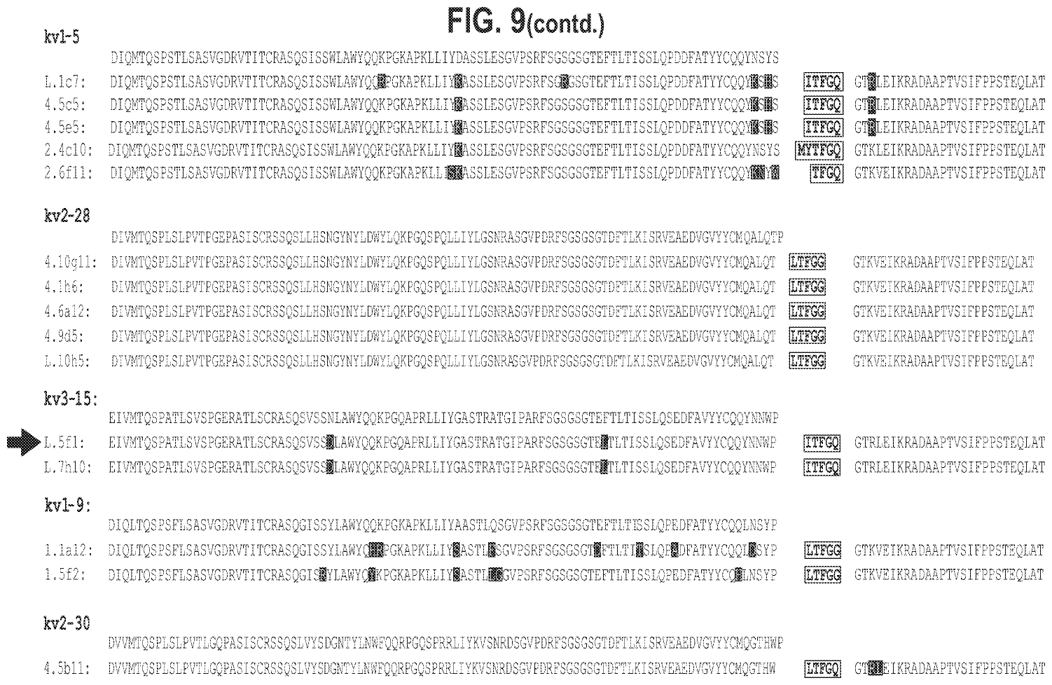

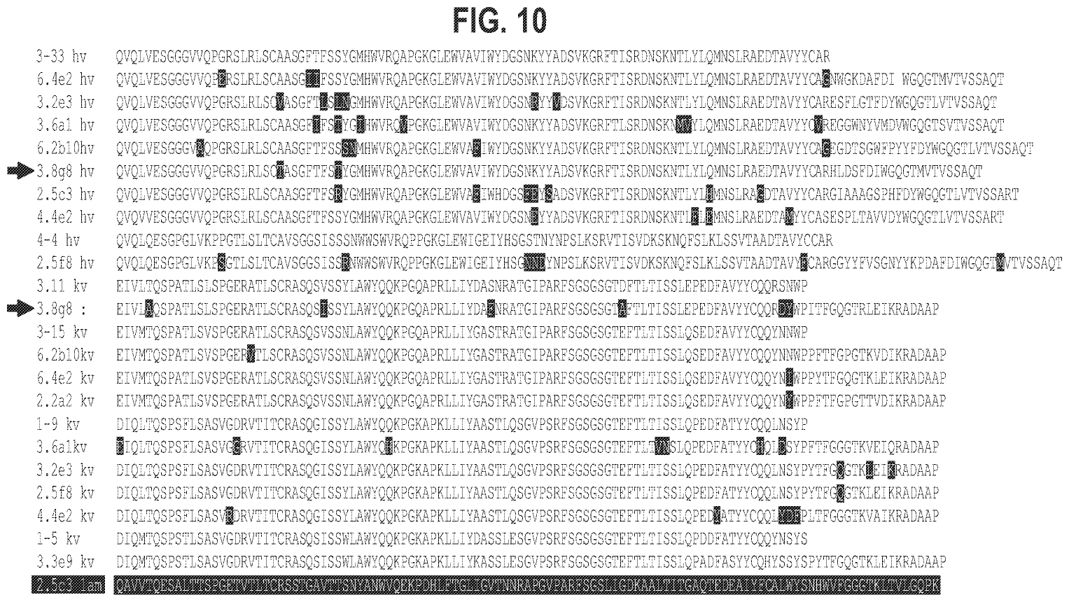

Hybridoma supernatants were also tested by FACs analysis on CD34− and Gpr56-expressing cell lines, and those with best affinities (3.8g8 and LF51, FIGS. 9 and 10) were selected for sub-cloning and sequencing. For this purpose, sub-cloned hybridomas were cultured in serum- and protein-free media for hybridoma culturing (PFHM-II (1×), Gibco) with addition of non-essential amino acids 100× NEAA, Biowhittaker Lonza, Catalog #BE13-114E). H2L2 antibodies were purified from hybridoma culture supernatants using Protein-G affinity chromatography (Temecula, California Cat. N 16-266). Purified antibodies were stored at 4° C. until use.

Sequencing of hybridomas was done according to Harbour protocol for H2L2 mice.

For recombinant human mAb production, variable regions of the heavy and light chains were cloned into expression plasmids containing the human IgG1 heavy chain and Ig kappa light chain constant regions, respectively (HarbourBioMed vectors pHBM000267 and pHBM000265). Both plasmids contained the mouse Ig kappa leader sequence to enable efficient secretion of recombinant antibodies. Recombinant human Abs 3.8g8 (anti-Gpr56), L5F1 (anti-CD34) and an anti-strep-tag Ab and anti-MSLN used for isotype controls were produced in HEK-293T cells following co-transfection of two expression plasmids encoding for appropriate IgG1 heavy and kappa light chains. Human antibodies were purified from cell culture supernatants using Protein-A affinity chromatography, eluted in 3M KSCn. 30 kD cut off Amicon filters were used for PBS buffer exchange and concentrating the sample. Concentrations were measured by nanodrop (280 nm) and antibodies were run on SDS page in reducing and non-reducing conditions. Purified antibodies were stored at 4° C. until use. Mice were immunized according to the protocol approved by the Dutch Experimental animal committee DEC Nr EUR 1944.

PLGA-NP Preparation

NPs with entrapped DiD fluorescent dye were prepared using an o/w emulsion and solvent evaporation-extraction method [10, 11]. In brief, 90 mg of PLGA in 3 mL of DCM containing the 3 mg DiD was added dropwise to 25 mL of aqueous 2% (w/v) PVA and emulsified for 90 s using a sonicator (Branson,sonofier 250). A combination of lipids (DSPE-PEG(2000)-maleimide (8 mg) and DSPE-PEG 2000 (8 mg) or (DSPE-PEG(2000)-NHS (8 mg) and DSPE-PEG 2000 (8 mg) were dissolved in DCM and added to the vial. The DCM was removed by a stream of nitrogen gas. Subsequently, the emulsion was rapidly added to the vial containing the lipids and the solution was homogenized during 30 s sonication. Following overnight evaporation of the solvent at 4° C., the PLGA-NPs were collected by ultracentrifugation at 60000 g for 30 min, washed three times with distilled water, and lyophilized.

Antibody Conjugation

1 mg of PLGA-PEG-DSPE-maleimide-NPs were resuspended in PBS and 50 μg of recombinant mAb was added. The mixture was incubated overnight at 4° C. or for 1 hour at room temperature under constant agitation in a tube rotator. Excess mAbs were removed by centrifugation at 25000 g for 30 min. Reactive maleimide groups on the NP-surface were quenched at the end of a reaction by adding free thiols (cysteine), followed by two washing steps with PBS.

Physicochemical Characterization of Antibody-Coated PLGA-NPs

Z-average size, polydispersity index (PDI) and zeta potential of antibody-coated-PLGA-PEG-NPs were measured using a Malvern ZetaSizer 2000 (Malvern, UK; software: ZetaSizer 7.03). Fixed scattering angle of 90° at 633 nm was set up for the analysis. DLS and zeta potential measurements were performed on PLGA-NPs freshly coated with mAbs.

Transmission Electron Microscopy

NP size and shape were characterized by transmission electron microscopy (TEM). Due to the low electron density of organic samples, such as PLGA-NPs, a negative staining was first performed to enhance the contrast. To this end, 3 μl antibody-coated-PLGA-NPs reconstituted in water (3 mg/ml) was deposited onto a carbon coated grid. PLGA-PEG-NPs were allowed to settle for approximately one minute, blotted dry, and then covered with a drop of 2.3% uranyl acetate. After one minute, this drop was also blotted dry, and the sample was ready for TEM analysis. The analysis was performed with a Tecnai 12 Biotwin (FEI, Oregon, US).

Cell Culture

Jurkat, 32D and 32D-gpr56 cells were cultured in RPMI-1640 medium supplemented with 10% FCS and 1% Pen/Strep. Peripheral blood mononuclear cells (PBMCs) were isolated from healthy buffy coat donors (Sanquin blood bank) by density centrifugation using Ficoll and directly used in targeting experiments.

Flow Cytometry

Isolated control and NP-treated PBMCs were resuspended in FACS buffer (2.5 g BSA in 500 ml PBS, 0.02% sodium azide) containing a 7-color panel of antibodies against hematopoietic stem and progenitor cell markers (HSPCs) (CD10, CD135, CD45RA, CD34, CD90, CD38, CD45), as well as the viability dye 7AAD. Only viable cells were included for analysis. Monocytes and lymphocytes could be gated based on their morphology within the CD45+ population. HSCs were CD34+CD45lowCD38−CD90+CD45RA− and multipotent progenitors (MPP) were CD34+CD45lowCD38−CD90−CD45RA−.

CD34+CD45lowCD38+CD10+ events were common lymphoid progenitor cells (CLPs). The CD38+CD10− populations contained common myeloid progenitor cells (CMPs, CD34+CD38+CD135+CD45RA−), granulocyte macrophage progenitor cells (GMPs, CD34+CD38+CD135+CD45RA+), and megakaryocyte erythroid progenitor cells (MEPs, CD34+CD38+CD135−CD45RA−). The percentage of NP-positive cells within different HSPC populations was assessed using a BD LSR-II. NP-uptake was assessed by measuring the percentage of DiD-positive cells within each subpopulation compared to control cells.

NP-Uptake Experiments

To assess NP-binding and uptake in PBMCs, the cells were washed and resuspended at 0.5×10⁶/500 μl in IMDM medium and incubated for 15 minutes at 37° C. with 20 μg/ml of antibody-conjugated PLGA-PEG-NPs. The cells were washed three times in 10 ml PBS and subsequently analysed by flow cytometry. Viable blood cells (7-AAD−) were gated as CD34+ and CD34− cells, or HSPC populations were further analysed using the markers CD10, CD135, CD45RA, CD34, CD90, CD38, CD45. Within each population, the percentage of NP(DiD)-positive cells was assessed.

To dissect the percentage of NPs that were bound from those that were taken up by PBMCs, PBMCs were washed and resuspended at 0.5×10⁶/500 μl in IMDM medium and incubated for 60 minutes at 37° C. (uptake and binding) or 4° C. (binding) with 20 μg/ml of antibody-conjugated PLGA-PEG-NPs. The cells were washed three times in cold PBS and subsequently analysed by flow cytometry.

Isolated CD34+ cells were washed and incubated for 15 minutes at 37° C. with 20 μg/ml of antibody-conjugated PLGA-PEG-NPs. Subsequently, the cells were washed three times in PBS and analysed by flow cytometry.

Confocal Microscopy

Uptake of antibody-conjugated PLGA-PEG-NPs by PBMCs and isolated CD34+ cells was analysed by confocal microscopy. To this end, PBMCs were incubated with 40 μg/ml antibody-conjugated PLGA-PEG-NPs for 1 hour at 37° C. and subsequently washed in PBS. The cell membrane was labelled using 10 μg/ml anti-CD34 primary antibody, followed by detection with a secondary antibody. After labelling, the cells were seeded on cover slips coated with 100 μg/ml poly-L-Lysine (Sigma Aldrich) for 15 min at RT. Finally, the cells were labelled with DAPI for 5 minutes at RT, fixed in 1% PFA for 15 min and embedded in mounting medium containing Mowiol and Dabco (Sigma Aldrich). Fluorescence imaging was performed with a SP5 confocal microscope (Wetzlar, Germany) using a 63× oil objective.

Methylcellulose

A methylcellulose colony-forming assay was performed as previously described [12]. PBMCs were stained for CD34 or Gpr56, or double stained for CD34 and Gpr56, and different populations of CD34− and Gpr56-expressing cells were sorted using a BD Aria-1. Sorted cells were centrifuged and resuspended in IMDM medium. Per dish, 700 sorted cells were seeded in triplicate in 3.6 ml methylcellulose (1 mL per dish; H4434; Stem Cell Technologies) with 1% PS and incubated for 14 days at 37° C., 5% CO2. Colonies were characterized and counted with a bright field microscope.

### Example 2

The aim of this study was to investigate the ability of PLGA nanoparticles (NPs) to deliver a CRISPR-Cas9 gene editing system to cells. CRISPR-PLGA-NPs were successfully synthesized and readily taken up by HUDEP-2 cells, primary erythroblasts and CD34+ cells, shown to be non-toxic and to increase the levels of HbF significantly. The development of CRISPR-PLGA-NPs to raise HbF levels in erythroid cells increases the toolbox of CRISPR delivery vehicles to target HSPCs for the treatment of hemoglobinopathies and other genetic diseases.

Materials

For the preparation of NPs, dichloromethane (DCM) and dimethylformamide (DMF) were purchased from Sigma-Aldrich® (Zwijndrecht, The Netherlands). Poly (D, L-lactic-co-glycolic acid) 50:50 (PLA/PGA), polyvinyl alcohol (PVA), calcium nitrate (Ca(NO3)2) and diammonium hydrogen phosphate (NH4)2HPO4 were purchased from Sigma-Aldrich. Cy5 Acid was purchased from Kerafast (Boston, US). Alt-R® Cas9 Nuclease V3 (S. pyogenes), Alt-R® CRISPR-Cas9 crRNA, Alt-R® CRISPR-Cas9 tracrRNA, Alt-R® CRISPR-Cas9 tracrRNA-ATTO™ 550 and nuclease-free water were purchased from Integrated DNA Technologies (IDT), (lowa, US). Chemically modified sgRNAs were purchased from Biolegio (Nijmegen, The Netherlands). Lipofectamine™ CRISPRMAX™ Cas9 Transfection Reagent was purchased from ThemoFisher Scientific (Massachusetts, US).

For cell culture, StemSpan and MethoCult™ H4434 Classic medium were purchased from STEMCELL Technologies (Vancouver, Canada). EPO Eprex (10001E) was purchased from Janssen-Cilag AG (Zug, Germany). Human recombinant SCF was purchased from BioLegend (San Diego, US). Dexamethasone, SyntheChol and doxycycline were purchased from Sigma Aldrich.

For flow cytometry, cells were stained with anti-HbF-FITC (clone REA533; Miltenyi Biotec, Bergisch Gladbach, Germany) anti-GPA (clone HIR2; BioLegend, California, USand anti-CD71 (clone CY1G4, BioLegend). Unconjugated antibodies were detected with the secondary antibody goat anti-mouse IgG (H+L)-Alexa Fluor 488 (Invitrogen, ThermoFisher Scientific). For confocal microscopy, cells were stained with anti-GPA (BioLegend), anti-Cas9 (clone 7A9, BioLegend), anti-CD34 (clone 561, BioLegend) anti-EEA-1 (polyclonal, Invitrogen, ThermoFisher Scientific). Secondary antibodies were donkey anti-rabbit Alexa-Fluor 488, goat anti-mouse IgG1 Alexa Fluor 488, Secondary antibodies were goat anti-mouse IgG2a Alexa Fluor 488 anti-mouse IgG1 Alexa Fluor 568 (Invitrogen, ThermoFisher Scientific). Lysosomes were labelled with LysoTracker™ Green (Invitrogen, ThermoFisher Scientific).

qRNAs

sgRNAs (Biolegio) were purchased as oligonucleotides. eGFP-targeting sgRNA: GGG CGA GGA GCU GUU CAC CG (SEQ ID NO: 29); sgRNA-2 [9]: CTT GTC AAG GCT ATT GGT CA (SEQ ID NO: 30); scrambled sgRNA (Biolegio): CCCGCUCCUCGACAAGUGGC (SEQ ID NO: 31).

CRISPR/Cas9-PLGA-Nanoparticle Preparation

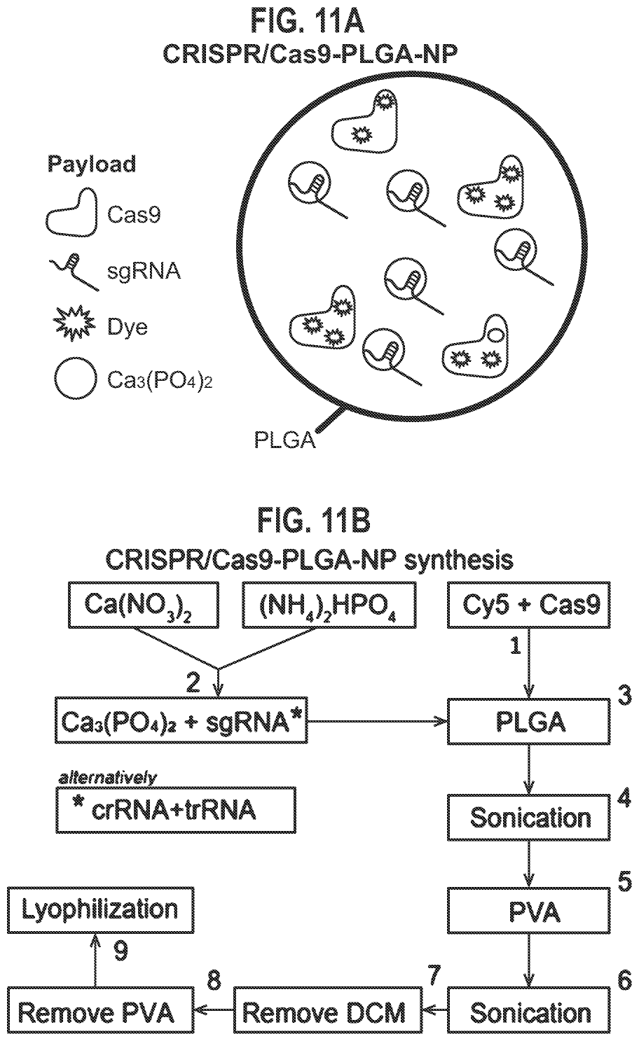

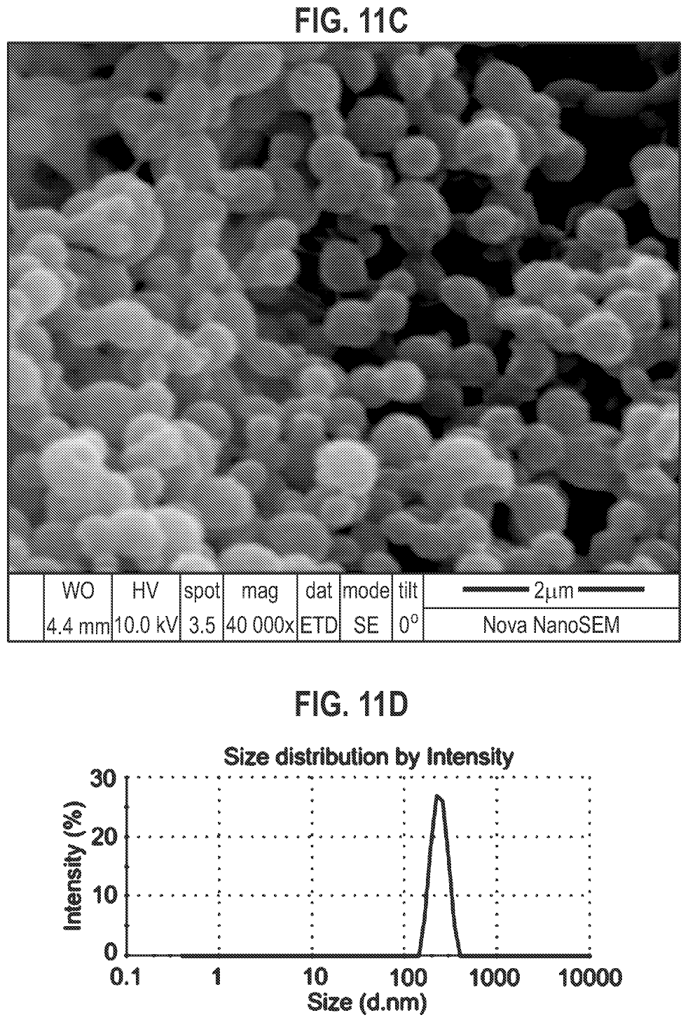

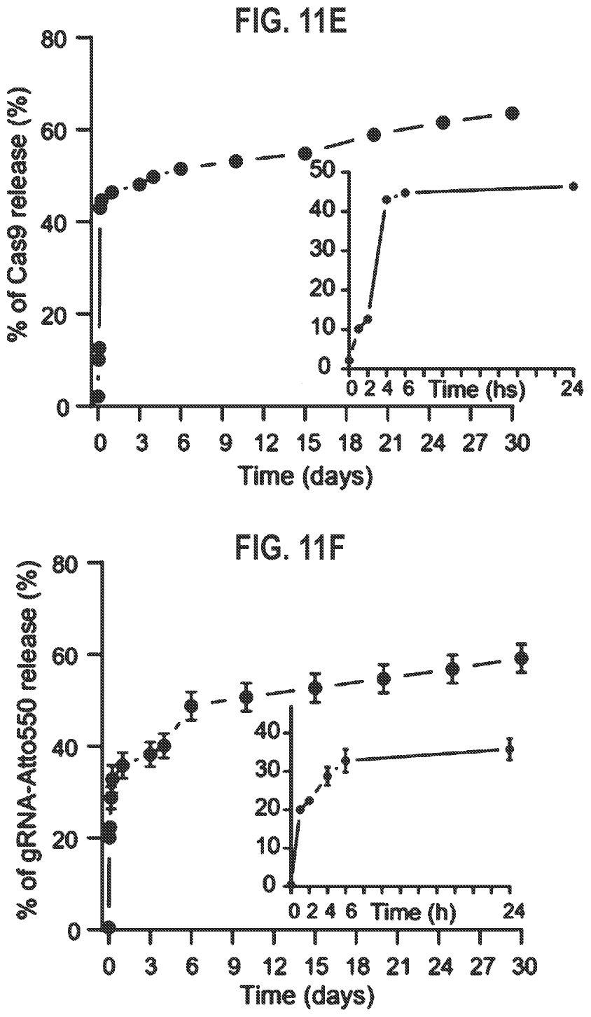

CRISPR/Cas9-PLGA-NPs were synthesized according to an oil-in-water (W1/O/W2) double emulsion solvent evaporation method [10, 11] (FIG. 11B). Prior to synthesis, beakers, spatulas, sonicator tip and magnetic stirrers were cleaned, decontaminated with RNAse-zap solution and rinsed with RNAse-free water. Firstly, a complex of Cy5-dye and Cas9-protein was prepared (1). To this end, 0.1 mg Cy5 (acid) dye was dissolved in 40 μl of RNAse free water and then 66 μg of Cas9 was added. The mixture was incubated for 30 minutes at room temperature (RT). Secondly, a solution of calcium phosphate was prepared to precipitate the gRNA (2). To this end, a rapid precipitation method that was reported earlier was modified [13]. Two pipettes were prepared, one containing 105 μl of aqueous calcium nitrate (6.25 mM) and another one with 105 μl of aqueous di-ammonium hydrogen phosphate (3.74 mM). Both solutions were simultaneously pipetted and mixed on top of a paraffin sheet. The resulting calcium phosphate solution was added dropwise into an Eppendorf tube containing 12.8 μg sgRNA or hybridized crRNA-tracerRNA complex (according to the IDT user manual) in 40 μl of duplex buffer, while vortexing. The mix of sgRNA and salt was cooled on ice for 5 minutes. In the next step the calcium phosphate-CRISPR/Cas9-PLGA-NPs was synthesized: 10 mg of PLGA were dissolved in 750 μl DCM (3). First, the 210 μl of calcium phosphate and gRNA prepared in step 2 were added. Secondly, the 40 μl of Cy5-Cas9-complex prepared in step 1 were added. The mixture of calcium phosphate-gRNA, Cy5-Cas9 and PLGA-polymer was immediately emulsified under sonication (Sonifier 250, ultrasonic tip Branson, Connecticut, US) using the following settings: 60 sec sonication time, constant duty cycle, output control: 4. During sonication, the mixture was kept on ice. Immediately after sonication, the W1/O phase was added dropwise into an aqueous phase (3000 μl) containing 30 mg of the emulsifier PVA. Prior to use, the aqueous phase of PVA was dissolved in a water bath at 80° C. for 20 min and cooled to RT. The solution was immediately sonicated as described above. After sonication, DCM was removed under reduced pressure (200-600 mbar) in a rotary evaporator for ˜2 min and the excess of PVA was removed by centrifugation (15 min at 14,800 rpm at 4° C.). Following centrifugation, the NPs were washed three times with 1000 μl RNAse-free water and subsequently freeze-dried. All washing solutions were stored to determine the loading efficacy of Cas9, Cy5 and gRNAs. The NPs were stored at −80° C. and rehydrated prior use.

Physicochemical Characterization of CRISPR/Cas9-PLGA-NPs

Z-average size, polydispersity index (PDI) and zeta potential of CRISPR/Cas9-PLGA-NPs were measured using a Malvern ZetaSizer 2000 (Malvern, UK; software: ZetaSizer 7.03). Fixed scattering angle of 90° at 633 nm was set up for the analysis. DLS and zeta potential measurements were performed on CRISPR/Cas9-PLGA-NPs resuspended in RNAse-free water before freeze-drying.

Transmission Electron Microscopy

NP size, shape and homogeneity were further characterized by transmission electron microscopy (TEM). Due to the low electron density of organic samples, such as PLGA-NPs, a negative staining was first performed to enhance the contrast. To this end, 3 μl CRISPR/Cas9-PLGA-NPs reconstituted in RNAse-free water (3 mg/ml) were deposited onto a carbon coated grid. CRISPR/Cas9-PLGA-NPs were allowed to settle for approximately one minute, blotted dry, and then covered with a drop of 2.3% uranyl acetate. After one minute, this drop was also blotted dry, and the sample was ready for TEM analysis. The analysis has been performed with a Tecnai 12 Biotwin (FEI, Oregon, US).

Quantification of Loading Efficacy into CRISPR/Cas9-PLGA-NPs

Cy5 loading efficacy into CRISPR/Cas9-PLGA-NPs was indirectly determined by quantifying Cy5 in the supernatants of the washing steps collected during NP-preparation using a spectrofluorometer, plotted against a Cy5 standard curve of known concentrations.

To quantify the amount of encapsulated gRNA, CRISPR/Cas9-PLGA-NPs encapsulating Cas9 and a hybridized gRNA consisting of crRNA and Atto550-labeled tracerRNA were prepared. gRNA loading efficacy into CRISPR/Cas9-PLGA-NPs was indirectly determined by quantifying the gRNA content in the supernatant of the washing steps collected during NP preparation. Supernatants and a standard curve of crRNA-tracerRNA-Atto550 with known concentrations were measured using a spectrofluorometer.

Cas9 loading efficacy into CRISPR/Cas9-PLGA-NPs was determined by nanodrop measurement. To this end, the amount of Cas9 protein in the supernatant of the washing steps, collected during NP preparation, was quantified. Per batch of NPs (10 mg PLGA), 66 μg Cas9 were added during synthesis. Due to the low concentration of Cas9 in the collected supernatants of the washing steps, all supernatants were first pooled and the volume was concentrated to 100 μl using Amicon® Ultra-2 Centrifugal Filters (Sigma Aldrich) with a molecular weight cut-off of 100.000 kDa. The concentrated washing solutions were measured using a nanodrop at 280 nm and blanked against washing solutions of control NPs synthesized without addition of Cas9. Successful encapsulation of Cas9 was further confirmed by SDS and Western Blot analysis of CRISPR/Cas9-PLGA-NPs hydrolysed overnight with 0.8M NaOH at 37° C.

In Vitro Release Kinetics of Cas9 and qRNA

To follow in vitro Cas9 and gRNA release, CRISPR/Cas9-PLGA-NPs encapsulating Cas9 and gRNA-Atto550 were resuspended in PBS at a concentration of 3.5 mg/ml. 300 μl of NP-solution (in triplicate) were placed in an Eppendorf tube and incubated at 37° C. on a heating block in shaking mode (400 rpm). At the indicated timepoints (0 h, 1 h, 2 h, 4 h, 6 h, 24 h, 48 h, 72 h, 96 h, 6 days, 10 days, 15 days, 20 days, 25 days and 30 days), 150 μl sample was withdrawn and the volume was replaced with 150 μl fresh PBS. After collecting the final timepoint, the samples and a gRNA-Atto550 standard curve were first measured using a spectrophotometer to quantify the amount of released gRNA. Next, the samples were concentrated to a volume of 20 μl using Amicon® Ultra-2 Centrifugal Filters and the amount of released Cas9 was determined by nanodrop measurement

The cumulative release was calculated according to equation (1):

E(%)=(VEΣ1n−1Ci+V0Cn)/m0×100 (1)

where E(%) is the cumulative release, VE is the withdrawn volume (150 μl), V0 is the begin volume, Ci and Cn are the Cas9 and gRNA concentrations, i and n are the sampling times and m0 is the total amount of Cas9 or gRNA loaded CRISPR/Cas9-PLGA-NPs.

Cell Culture

Human umbilical cord blood-derived erythroid progenitor-2 (HUDEP-2) were cultured as described previously [14]. Briefly, HUDEP-2 cells were cultured in StemSpan (Stem Cell Technologies) serum-free expansion medium supplemented with 1 μM dexamethasone, 1 μg/ml doxycycline, 50 ng/ml human SCF, 2 units/ml EPO, 0.4% SyntheChol and 1% penicillin-streptomycin. Differentiation of HUDEP-2 cells was initiated by removal of doxycycline, dexamethasone and SCF, and an increase in EPO to 10 units/ml. HUDEP-2-eGFP expressing cells were generated by lentiviral transduction of HUDEP-2 cells with pRRL-CMV-GFP plasmid (kind gift of M.J.W.E. Rabelink, LUMC). One week after transduction, eGFP-high-expressing HUDEP-2 cells were sorted using a BD (New Jersey, US) FACSARIA I flow cytometer and the bulk of eGFP-expressing cells was further propagated. As a positive control, gRNA/Cas9 RNP complex were delivered to WT HUDEP-2 cells by electroporation using the Neon transfection system (ThermoFisher Scientific). The following settings were used during electroporation: 1600 V, 10 ms, 3 pulses. Electroporated HUDEP-2 were propagated and used as a control.

Peripheral blood mononuclear cells (PBMCs) were obtained from healthy buffy coat donors (Sanquin blood bank) and were cultured according to a three-phase erythroid differentiation protocol in StemSpan serum-free expansion medium supplemented with 1 μM dexamethasone, 50 ng/ml human SCF, 2 units/ml EPO, 0.4% SyntheChol, and 1% penicillin-streptomycin [15, 16]. During phase 1 (days 1-7), 1 ng/ml human interleukin (IL)-3 (BioLegend) and 40 ng/ml human insulin-like growth factor I (IGF) (BioLegend) were included. Phase 2 (days 8-12) included the same medium, except that IL-3 and IGF1 were withdrawn. Erythroid differentiation was monitored by flow cytometry using anti-CD71 and anti-GPA antibodies.

CRISPR/Cas9-PLGA-NPs-Mediated Gene Editing

1×10⁵ HUDEP-2 cells were resuspended in 100 μl StemSpan medium (including supplements) and plated into a 96-well (flat bottom) plate. Lyophilized CRISPR/Cas9-PLGA-NPs were resuspended at 5 mg/ml in RNAse-free water and immediately further diluted in StemSpan medium. CRISPR/Cas9-PLGA-NPs were kept at all time on ice. 100 μl of CRISPR/Cas9-PLGA-NPs were added to 100 μl of HUDEP-2 cells to reach a final concentration of 200 μg/ml, 100 μg/ml, 50 μg/ml, 25 μg/ml or 12.5 μg/ml. HUDEP-2 cells were incubated with CRISPR/Cas9-PLGA-NPs or control-NPs for 24 hours at 37° C., subsequently washed with medium to remove excessive NPs and resuspended in fresh medium. NP-treated HUDEP-2 were cultured up to 21 days after NP-treatment. The cells were split and the medium was refreshed every three days. At designated timepoints, cells were withdrawn for flow cytometry and RNA analysis. Samples were prepared in triplicate.

Primary erythroblasts were edited at the end of phase 1 using the erythroid differentiation protocol. At day 8, when the cell culture was populated by erythroid progenitors, the cells were collected and 1×10⁵ cells were resuspended in 100 μl phase 2-medium and plated into a 96-well (flat bottom) plate, and treated with 200 μg/ml, 100 μg/ml, 50 μg/ml, 25 μg/ml or 12.5 μg/ml CRISPR/Cas9-PLGA-NPs or control-NPs, as described above. The cells were expanded and cultured in phase-2 medium until day 21. At designated timepoints, cells were withdrawn for flow cytometry and RNA analysis.

Flow Cytometry

HUDEP-2 cells, primary erythroblasts or isolated CD34+ cells were collected and washed in PBS. To assess cell viability after NP-treatment, cells were resuspended in FACS buffer (2.5 g BSA in 500 ml PBS, 0.02% sodium azide) containing Hoechst. The percentage of Hoechst positive/negative cells was assessed using a BD LSR-II equipped with a UV-laser. NP-uptake was assessed by measuring the percentage of Cy5-positive cells compared to non-treated control cells.

To determine the expression of HbF in HUDEP-2 cells or primary erythroblasts, cells were fixed, permeabilized and stained using an intracellular labelling kit (Inside Stain Kit, Miltenyi Biotec) and anti-HbF-Fitc antibody (Miltenyi Biotec) according to manufactures instructions.

Confocal Microscopy

Uptake and intracellular routing of CRISPR/Cas9-PLGA-NPs in HUDEP-2 cells were analysed by confocal microscopy. To this end, HUDEP-2 cells were incubated with 200 μg/ml CRISPR/Cas9-PLGA-NPs for 1, 4 and 24 hours at 37° C. and subsequently washed in PBS. The cell membrane was labelled using 10 μg/ml anti-GPA primary antibody, followed by detection with a secondary antibody. After labelling, the cells were fixed with 4% paraformaldehyde in PBS for 15 min at RT, washed and permeabilized with 0.1% Triton in PBS for 5 min. Intracellular labelling was performed by incubating the cells with 10 μg/ml anti-EEA-1 or anti-Cas9 antibody, followed by incubation conjugated secondary antibody. Cells were washed and seeded on cover slips coated with 100 μg/ml poly-L-Lysine (Sigma Aldrich) for 15 min at RT. Finally, the cells were labelled with DAPI for 5 minutes at RT, fixed in 1% PFA for 15 min and embedded in mounting medium containing Mowiol and Dabco (Sigma Aldrich). For labelling of lysosomes, cells were extensively washed and incubated with green lysotracker (ThermoFisher Scientific) for 30 minutes at 37° C. prior to cell membrane labeling. Fluorescence imaging was performed with a SP5 confocal microscope (Wetzlar, Germany) using a 63× oil objective.

RT-qPCR

Edited HUDEP-2 and control cells were washed with PBS and lysed in the cell culture plate using TRIzol (Invitrogen, Grand Island, NY, USA). Total RNA content was isolated according to manufacturer's protocol. Reverse transcription was performed using the M-MLV Reverse Transcriptase (Promega, Madison, WI, USA). Expression levels of GAPDH, EGFP, HBB and HBG were analysed using real-time, quantitative PCR. All real-time PCR reactions were performed using the Real-Time PCR Detection System from Biorad and all amplifications were performed using SYBR Green and PlatinumTaq (Thermofisher Scientific). The quality of the products was confirmed by melting curve analysis. The following expression primers were used: forward (F) primer CATTGCCCTCAACGACCACT (SEQ ID NO: 19) and reverse (R) primer GGTGGTCCAGGGGTCTTACT (SEQ ID NO: 20) for GAPDH, F primer GCCCTGGCCCACAAGTATC (SEQ ID NO: 21) and R primer GCCCTTCATAATATCCCCCAGTT (SEQ ID NO: 22) for HBB, F primer GGTGACCGTTTTGGCAATCC (SEQ ID NO: 23) and R primer GTATCTGGAGGACAGGGCAC (SEQ ID NO: 24) for HBG, F primer ATCTTCTTCAAGGACGACGG (SEQ ID NO: 25) and R primer GGCTGTTGTAGTTGTACTCC (SEQ ID NO: 26) for EGFP. Throughout this example, percent HBG mRNA refers to the abundance of HBG mRNA expressed relative to the sum of the abundance of γ-globin and β-globin transcripts ([HBG/(HBB+HBG]*100).

Methylcellulose

Methylcellulose colony-forming assay was performed as described previously [12]. CD34+ were isolated from PBMCs using human CD34+ MicroBeadKit (Miltenyi) and MS columns for positive selection of CD34+ cells. Isolated CD34+ cells were incubated with 200 μg/ml CRISPR/Cas9-PLGA-NPs or control-NPs in IMDM medium for 30 minutes at 37° C., 5% CO2. Subsequently, the cells were centrifuged and without washing seeded in methylcellulose. 500 CD34+ cells were seeded in triplicate in 3.6 ml methylcellulose (1 mL per dish; H4434; Stem Cell Technologies) with 1% PS and incubated for 14 days at 37° C., 5% CO2. Colonies were characterized and counted with a bright field microscope, and the Cy5 signal inside the colonies was obtained in a scan with the Odyssey Scanner (LI-COR, Nebraska, US) at 680 nm.

Sanger Sequencing and TIDE Analysis

Sanger sequencing was performed on BFU-E colonies grown for 14 days in methylcellulose. After characterization and counting, BFU-E colonies were harvested in a 96-well plate and washed with PBS. Subsequently, DNA was extracted using the Wizard Genomic DNA Purification Kit (Promega). 2 μl of the cell lysate was used as input in a PCR reaction using Phusion DNA polymerase (ThermoFisher Scientific) and the following primers: Forward ACGGCTGACAAAAGAAGTCC (SEQ ID NO: 27) and Reverse GGGTTTCTCCTCCAGCAT (SEQ ID NO: 28). PCR products were cleaned up using Wizard SV Gel and PCR clean-up system (Promega). TIDE analysis was performed on Sanger sequencing trace data to access indel frequency in BFU-E colonies using the online tool shinyapps.datacurators.nl/tide/ [17].

Results

CRISPR/Cas9-PLGA-NPs Synthesis and Characterization

To circumvent efficacy and safety issues of viral vehicles, PLGA-NPs encapsulating Cas9 (S. pyogenes) protein, gRNA and the fluorescent dye Cy5 were designed (FIG. 11A). The aim of this study was to synthesize a delivery vehicle for CRISPR that is efficiently processed by HSPCs and could be monitored by fluorescence microscopy. In the classical solvent evaporation technique, gRNAs can easily escape from the encapsulation process due to their low molecular weight, hydrophilicity and the electrostatic repulsion between the phosphate backbone of the RNA and the carboxylic acid end groups of the PLGA-building blocks [18]. To avoid this, a previously reported method where siRNA absorbed onto the surface of calcium phosphate was encapsulated into the hydrophobic core of PLGA was adjusted [13].

Here, CRISPR/Cas9-PLGA-NPs were synthesised according to an oil-in-water double emulsion solvent evaporation method (FIG. 11B). Prior to encapsulation, a complex of Cy5 acid (net charge −1) and Cas9 (net charge of +22) was prepared, based on electrostatic interaction (FIG. 11A-B). Cy5 acid dye contains a non-activated carboxylic acid; thus, the molecule is considered non-reactive. Forming a complex between Cas9 and Cy5 instead of directly conjugating Cy5 to the Cas9 protein would avoid loss of Cas9 functionality and increase the encapsulation efficacy of the Cy5 dye. Secondly, a solution of calcium phosphate was prepared to precipitate the gRNA (sgRNA or hybridized crRNA-tracerRNA complex) under formation of calcium phosphate/gRNA complexes. In the next step calcium phosphate-CRISPR/Cas9-PLGA-NPs were synthesised: PLGA was dissolved in DCM and then the calcium phosphate/gRNA- and Cas9/Cy5-in-water complexes were added. Sonication led to the formation of the first emulsion with phosphate/gRNA and Cas9/Cy5 complexes in-water in the oil phase. Addition of PVA (dissolved in water) and subsequent sonication led to the formation of a stable double oil-in-water emulsion. The organic solvent was removed under reduced pressure and the surfactant PVA under centrifugation. The resulting NPs were immediately freeze-dried.

A encapsulated chemically modified gRNA directed against the sequence of enhanced green fluorescent protein (eGFP) or a scrambled control sequence was encapsulated. The size of the NPs was determined by dynamic light scattering (DLS) analysis as ˜370 nm in diameter (Table 2, FIG. 11D) and the polydispersity index (PDI) values of 0.1-0.2 obtained from the DLS measurement indicated a homogeneous size distribution (Table 1). In addition, ZetaSizer measurement showed a negative surface charge of the NPs (Table 1). In contrast to control NPs without Cas9, encapsulation of Cas9 increased the size of the NPs from 215 to >300 nm (Table 1), indicating successful encapsulation of Cas9. Nanodrop analysis of the washing solutions generated during NP synthesis showed encapsulation efficacies of 49-75% for Cas9 and 69-89% for sgRNA. In addition, spectrophotometry measurements revealed that 26-65% of Cy5 dye was encapsulated (Table 2).

TABLE 2
Physiochemical characterisation of CRISPR/Cas9-NPs

| | NP | Zeta | | | | |
|---|---|---|---|---|---|---|
| | size ± | Potential ± | | | | |
| | S.D. | S.D. | | EE (%) | EE (%) | EE (%) |
| Sample | (nm) | (mV) | PDI | gRNA | Cas9 | Cy5 |
| PLGA-NP- | 214.7 ± | −16.0 ± | 0.22 ± | 83.85 | — | 26.29 |
| (guideRNA, Cy5) | 3.06 | 1.79 | 0.05 | | | |
| PLGA-NP-(Cas9, | 378.5 ± | −17.6 ± | 0.12 ± | 84.58 | 49.12 | 47.39 |
| “scrambled | 79.37 | 2.17 | 0.05 | | | |
| sgRNA”, Cy5) | | | | | | |
| PLGA-NP-(Cas9, | 362.4 ± | −25.4 ± | 0.21 ± | 88.91 | 70.91 | 64.78 |
| “eGFP-sgRNA”, | 46.68 | 4.4 | 0.05 | | | |
| Cy5) | | | | | | |
| PLGA-NP-(Cas9, | 307.67 ± | −13.8 ± | 0.23 ± | 78.52 | 75.01 | 32.63 |
| “eGFP-crRNA + | 2.45 | 1.42 | 0.14 | | | |
| tracerRNA”, Cy5) | | | | | | |
| PLGA-NP-(Cas9, | 296.67 ± | −9.49 ± | 0.15 ± | 69.92 | 75.17 | 60.68 |
| “γ-globin targeting | 101.04 | 0.9 | 0.06 | | | |
| sgRNA”, Cy5) | | | | | | |

To monitor the release kinetics of Cas9 from CRISPR/Cas9-PLGA-NPs, the NPs were incubated in PBS at 37° C. for 30 days and withdrew samples at designated timepoints. Cas9 was analysed by nanodrop measurement and the cumulative release was calculated (FIG. 11E). The gRNA release was calculated from a batch of CRISPR/Cas9-PLGA-NPs that encapsulated Atto-550-labeled gRNA (FIG. 11F). The release kinetic studies showed that 30% of the encapsulated gRNA and 40% of the Cas9 were rapidly released within the first 24 hours, followed by a period of sustained release lasting until the endpoint of analysis (FIG. 11E-F).

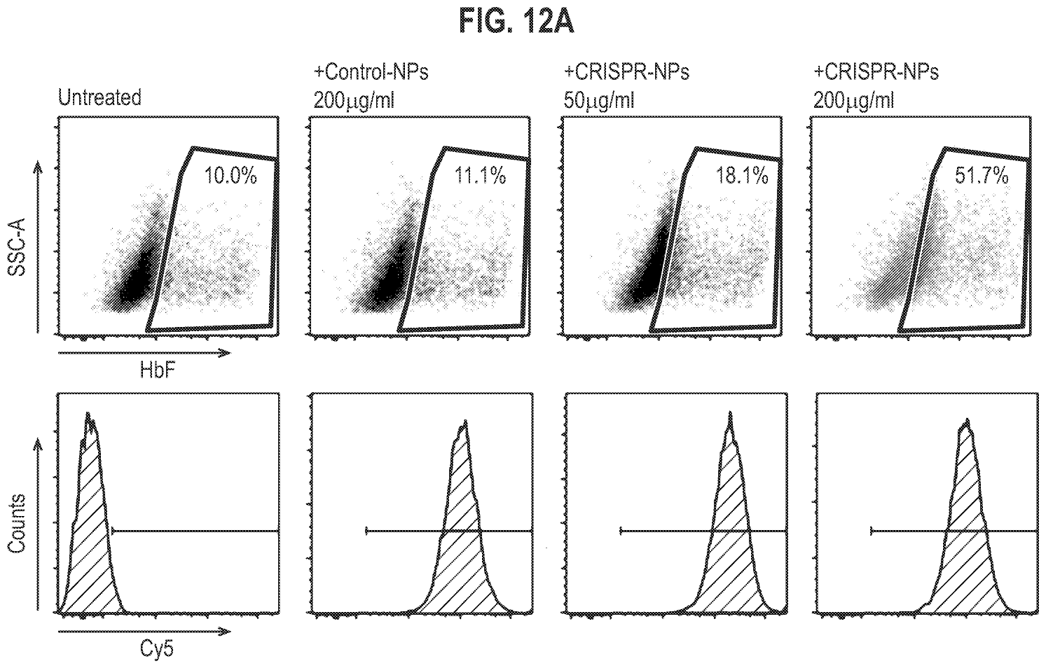

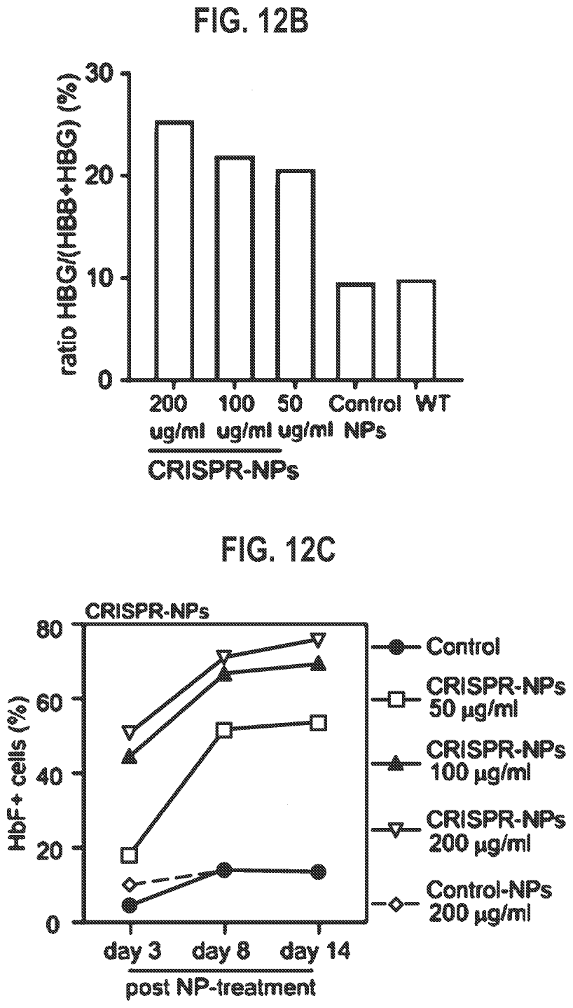

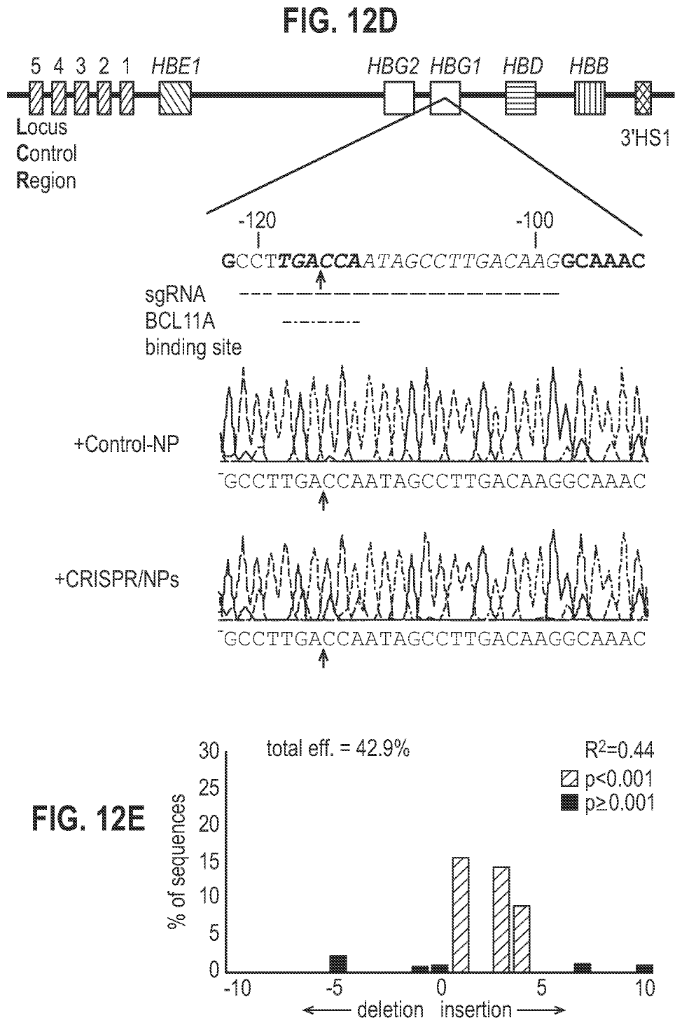

Next, uptake and gene-editing efficacy of CRISPR/Cas9-PLGA-NPs was assessed on primary human erythroblasts, cultured from PBMCs using an erythroid differentiation protocol. In the first phase, erythroid progenitors were expanded until they dominated the cell culture around day 8. Erythroid progenitors were incubated with different concentrations of CRISPR/Cas9-PLGA-NPs (encapsulating the γ-globin promoter targeting gRNA) and control-NPs, followed by induction of the expansion phase. Three days after NP-incubation (day 11 of erythroblast differentiation), the percentage of NP-positive cells and the intracellular levels of HbF was measured by flow cytometry (FIG. 12A). Similar to the results obtained in HUDEP-2 cells, NPs were readily taken up by erythroblast progenitors in a concentration dependent manner (FIG. 12A). 10% of primary cells expressed HbF and incubation with control-NPs did not increase HbF expression, despite efficient uptake of control-NPs (FIG. 12A). CRISPR/Cas9-PLGA-NPs elevated the levels of HbF in a concentration-dependent manner to 18.1% (50 μg/ml) and 51.7% (200 μg/ml) (FIG. 12A). RT-PCR confirmed a concentration-dependent increase in the percentage of HbF expression 3 days after treatment with CRISPR/Cas9-PLGA-NPs, but not with control-NPs (FIG. 12B). Analysis of HbF expression over time by flow cytometry showed that the levels of HbF further increased on day 8 and day 14 after treatment with CRISPR/Cas9-PLGA-NPs, while the levels of HbF in WT and control-NP-treated cells remained constant (FIG. 12C). 200 μg/ml and 100 μg/ml CRISPR-Cas9-PLGA-NPs induced high levels of HbF already on day 3, while at 50 μg/ml, increased levels of HbF could be detected on day 8. Genomic analysis of CRISPR/Cas9-PLGA-NPs and control-NP-treated erythroblasts confirmed mutations in the HBG promoter region (FIG. 12D). To estimate the frequency and kind of insertions/deletions (indels), TIDE analysis was performed [17] on the Sanger sequencing trace data of the bulk of edited erythroblasts (FIG. 12E). The total indel efficacy was 42.9%, and the majority of mutations were insertions. In summary, this data demonstrates the functionality and efficacy of CRISPR-Cas9-PLGA-NPs in inducing HbF in primary erythroblasts.

CRISPR/Cas9-PLGA-NP-Mediated Gene-Editing of Primary HSPC

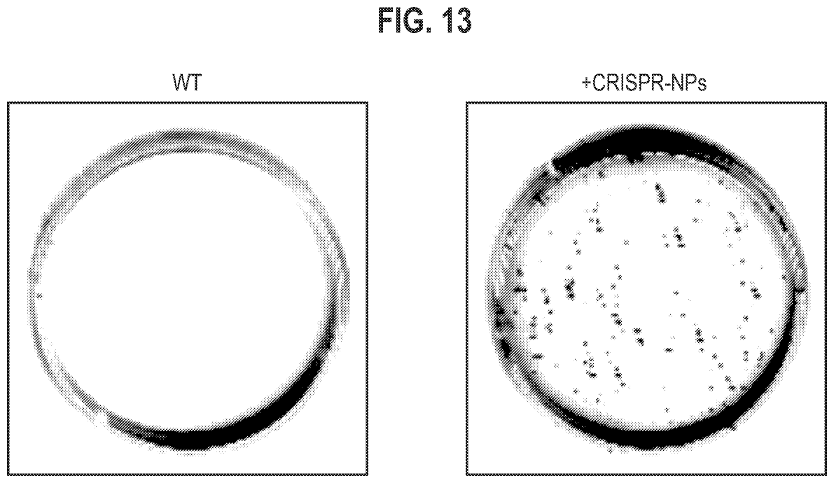

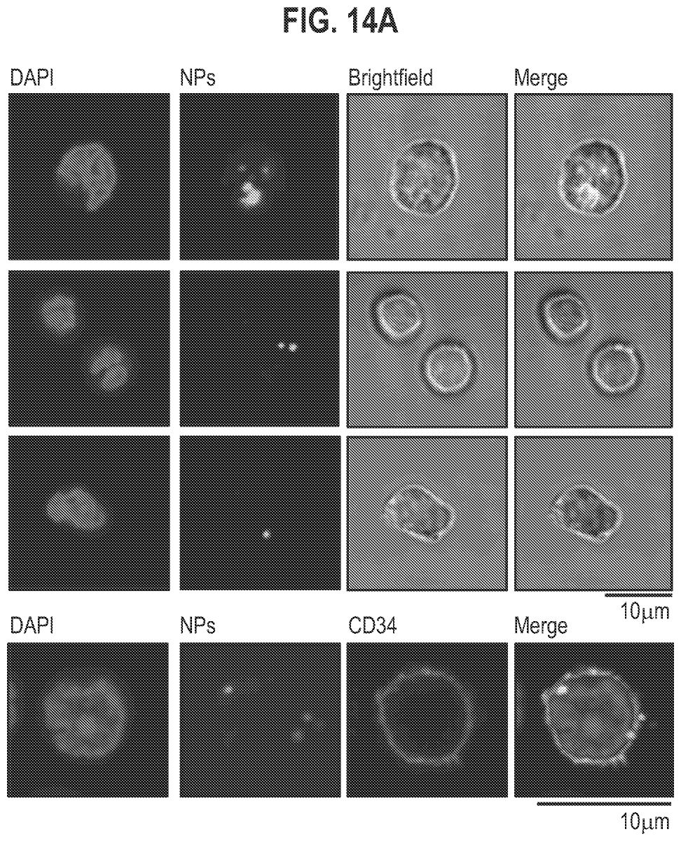

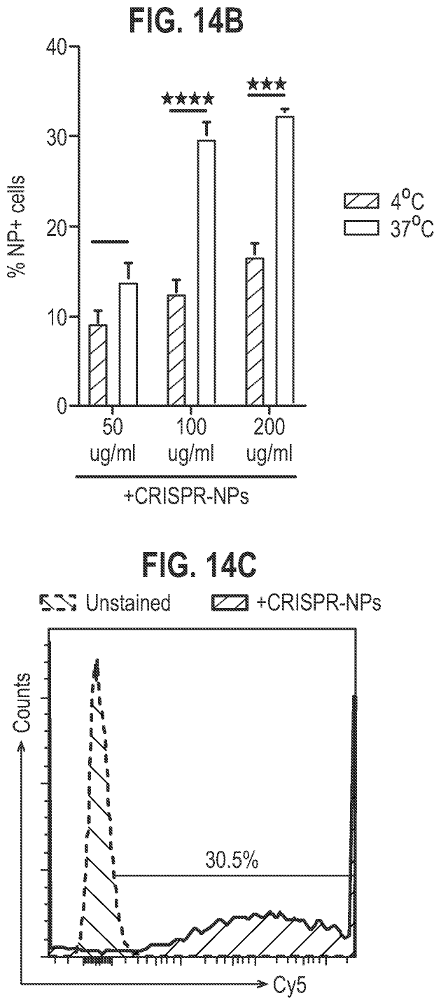

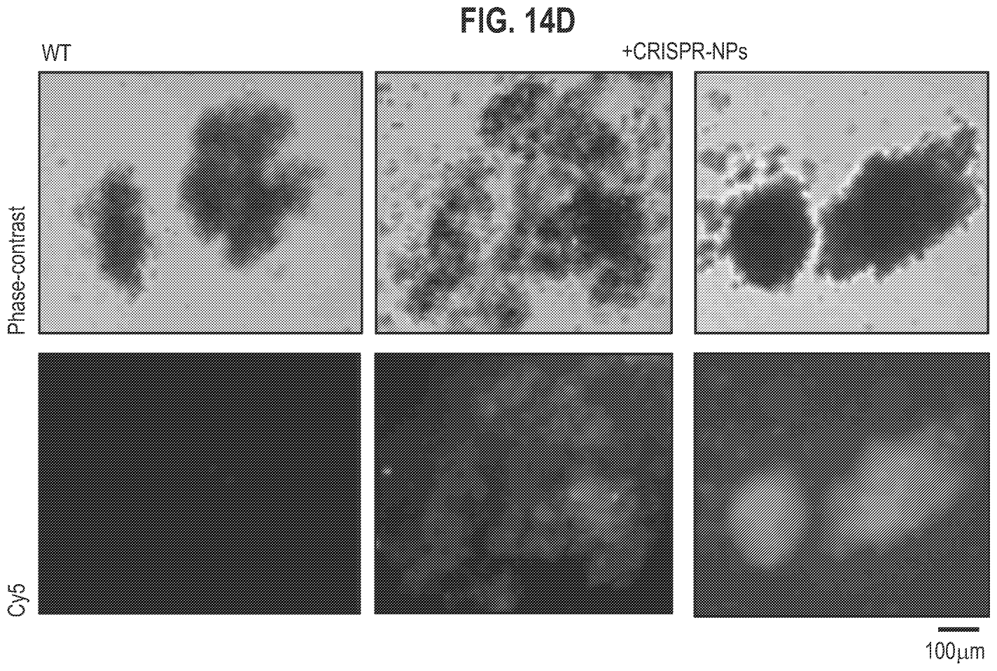

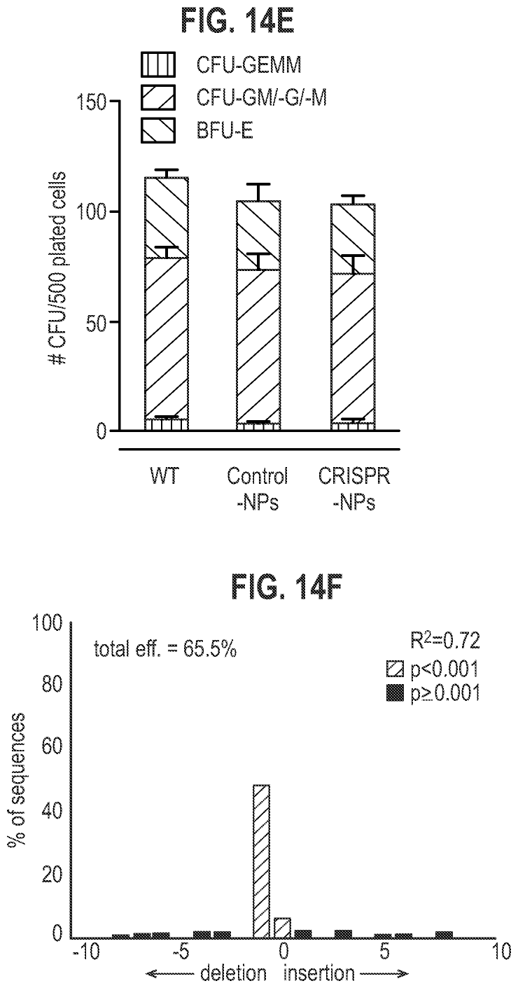

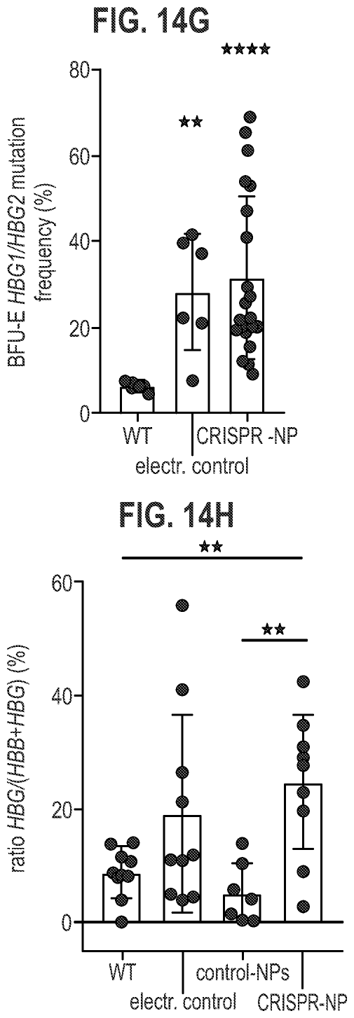

The application of CRISPR-Cas9 technology has been hampered by challenges in efficient non-viral expression and delivery of CRISPR components in CD34+ cells, in particular in HSPCs. To evaluate the efficacy of CRISPR/Cas9-PLGA-NPs as delivery system for the CRISPR-RNP complex to HSPCs, uptake studies were performed on isolated human CD34+ cells. Freshly isolated CD34+ cells from PBMCs were incubated with 100 μg/ml CRISPR/Cas9-PLGA-NPs for 1 hour at 37° C. and binding and uptake of NPs was evaluated by confocal microscopy (FIG. 14A). CRISPR/Cas9-PLGA-NPs bound and were taken up by different cell types within the population of CD34+ cells, including CD34high cells. Additional flow cytometry studies were performed to analyse the binding and uptake behaviour of CD34+ cells. To this end, CD34+ cells were incubated with CRISPR/Cas9-PLGA-NPs at 4° C., when cells can bind but not take up NPs, and at physiological conditions at 37° C. (FIG. 14B). Binding and uptake of CRISPR/Cas9-PLGA-NPs by CD34+ cells were concentration-dependent. Thus, for further analysis CD34+ cells were incubated with 200 μg/ml CRISPR/Cas9-PLGA-NPs. To evaluate potential cytotoxicity and the gene-editing capacity of CRISPR/Cas9-PLGA-NPs on CD34+ cells, clonal studies were performed on single-cell-derived burst-forming unit-erythroid (BFU-E) colonies grown in methylcellulose culture. To avoid differentiation, CD34+ cells were only shortly incubated with 200 μg/ml CRISPR/Cas9-PLGA-NPs. To increase NP-loading, CRISPR/Cas9-PLGA-NPs were centrifuged on CD34+ cells and NP-loaded cells were directly plated into methylcellulose. Flow cytometry confirmed efficient binding of CRISPR/Cas9-PLGA-NPs to CD34+ cells (FIG. 14C). To assess the number of Cy5+ colonies, the methylcellulose plates were scanned at 700 nm with the Odyssey imaging system (FIG. 13).

Almost all colonies derived from CD34+ cells treated with CRISPR/Cas9-PLGA-NPs were Cy5+, while there was no background signal in WT colonies. Further analysis of single clones showed that almost all cells within a colony were Cy5+(FIG. 14D). There was no difference in the number and type of hematopoietic progenitor colonies derived from WT, CRISPR/Cas9-PLGA-NP and control-NP treated isolated CD34+ cells (FIG. 14E). TIDE analysis of BFU-E colonies from CRISPR/Cas9-PLGA-NPs-treated and electroporated CD34+ cells revealed mutations in the γ-globin promoter region (FIG. 14F). Among the mutations, deletions and insertions were deleted. Analysed BFU-E colonies from CRISPR/Cas9-PLGA-NPs-treated and electroporated CD34+ cells showed on-target mutations. On average 32% and 28% of the screened BFU-E colonies derived from CRISPR/Cas9-PLGA-NP-treated and electroporated CD34+ cells, respectively, had indels and were mosaic for HBG1/HBG2 mutations (FIG. 14G). This most likely reflects editing over several rounds of cell division. RT-qPCR analysis of individual BFU-E colonies confirmed an increase in HbF mRNA expression in colonies derived from CD34+ cells treated with CRISPR/Cas9-PLGA-NPs and after electroporation (FIG. 14H). Thus, this data indicates that a functional CRISPR-complex was released for a prolonged period of time in CD34+ cells, resulting in highly efficient gene-editing without inducing cellular cytotoxicity.

### REFERENCES

1. Patra et al., “Nano based drug delivery systems: recent developments and future prospects”, J Nanobiotechnology. 2018; 16(1):71
2. Kumari et al., “Nanoencapsulation for drug delivery”, EXCLI J. 2014; 13: 265-286
3. Zhao and Stenzel, “Entry of nanoparticles into cells: the importance of nanoparticle properties”, Polymer Chemistry. 2018; 9(3):259-272
4. Deirram et al., “pH-Responsive Polymer Nanoparticles for Drug Delivery”, Macromol Rapid Commun. 2019; 40(10): e1800917
5. Hsu et al., “Development and Applications of CRISPR-Cas9 for Genome Engineering”, Cell. 2014; 157(6): 1262-1278
6. Brezgin et al., “Dead Cas Systems: Types, Principles, and Applications”, Int J Mol Sci. 2019; 20(23):6041
7. Whinn et al., “Nuclease dead Cas9 is a programmable roadblock for DNA replication”, Sci Rep. 2019; 9(1):13292
8. Wang et al., “Identification and characterization of essential genes in the human genome”, Science. 2015; 350(6264):1096-101
9. Traxler, et al., “A genome-editing strategy to treat beta-hemoglobinopathies that recapitulates a mutation associated with a benign genetic condition”, Nat Med. 2016; 22(9):987-90
10. Perez et al., “Poly(lactic acid)-poly(ethylene glycol) nanoparticles as new carriers for the delivery of plasmid DNA”, J Control Release. 2001; 75(1-2):211-24
11. Luo et al., “Controlled DNA delivery systems”, Pharm Res. 1999; 16(8):300-8
12. Medvinsky et al., “Analysis and manipulation of hematopoietic progenitor and stem cells from murine embryonic tissues”, Curr Protoc Stem Cell Biol. 2008; Chapter 2: Unit 2A 6
13. Dordelmann et al., “Calcium phosphate increases the encapsulation efficiency of hydrophilic drugs (proteins, nucleic acids) into poly(d,l-lactide-co-glycolide acid) nanoparticles for intracellular delivery”, J Mater Chem. 2014; B 2(41):7250-7259
14. Kurita et al., “Establishment of immortalized human erythroid progenitor cell lines able to produce enucleated red blood cells”, PLoS One. 2013; 8(3):e59890
15. Ronzoni et al., “Erythroid differentiation and maturation from peripheral CD34+ cells in liquid culture: cellular and molecular characterization”, Blood Cells Mol Dis. 2008; 40(2):148-55
16. van den Akker et al., “The majority of the in vitro erythroid expansion potential resides in CD34(−) cells, outweighing the contribution of CD34(+) cells and significantly increasing the erythroblast yield from peripheral blood samples”, Haematologica. 2010; 95(9):1594-8
17. Brinkman et al., “Easy quantitative assessment of genome editing by sequence trace decomposition”, Nucleic Acids Research. 2014; 42(22): e168
18. Cun et al., “High loading efficiency and sustained release of siRNA encapsulated in PLGA nanoparticles: quality by design optimization and characterization”, Eur J Pharm Biopharm. 2011; 77(1):26-35

1. An antibody conjugate comprising an antibody and a nanoparticle conjugated to the antibody, wherein the nanoparticle comprises a gene editing payload.

2. The antibody conjugate of claim 1, wherein the gene editing payload comprises a nuclease.

3. The antibody conjugate of claim 2, wherein the nuclease is an endonuclease that is capable of causing a single or a double strand break at a specific site in a genome.

4. The antibody conjugate of claim 2, wherein the nuclease is an endonuclease that is capable of causing a single or a double strand break at a non-specific site in a genome.

5. The antibody conjugate of claim 2, wherein the gene editing payload comprises one or more of: a meganuclease, a zinc-finger nuclease (ZFN), a transcription activator-like effector nuclease (TALEN), a clustered regularly interspaced palindromic repeats (CRISPR) nuclease.

6. The antibody conjugate of claim 1, wherein the gene editing payload comprises a nucleic acid that encodes one or more of: a meganuclease, a zinc-finger nuclease (ZFN), a transcription activator-like effector nuclease (TALEN), a clustered regularly interspaced palindromic repeats (CRISPR) nuclease.

7. The antibody conjugate of claim 2, wherein the gene editing payload comprises a CRISPR nuclease.

8. The antibody conjugate of claim 7, wherein the gene editing payload further comprises:
(a) a CRISPR RNA (crRNA) and a trans-activating crRNA (tracrRNA), or
(b) a guide RNA (gRNA).

9. The antibody conjugate of any one of claims 1-8, wherein the antibody targets a hematopoietic stem cell (HSC) surface antigen.

10. The antibody conjugate of claim 9, wherein the HSC surface antigen is one of CD34, Gpr56, Gpr97, CD49, CD49f, CD90, CD117 and endomucin.

11. The antibody conjugate of claim 10, wherein the HSC surface antigen is CD34.

12. The antibody conjugate of claim 10, wherein the HSC surface antigen is Gpr56.

13. The antibody conjugate of any one of claims 9-12, for use in treating a genetic blood disorder, congenital immunodeficiency or cancer.

14. The antibody conjugate for use of claim 13, wherein the gene editing payload is capable of repairing a genetic defect that contributes to the genetic blood disorder, congenital immunodeficiency or cancer.

15. The antibody conjugate of any one of claims 1-8, wherein the antibody binds to a cancer cell surface antigen.

16. The antibody conjugate of claim 15, wherein the cancer cell surface antigen is one of CD33, CD30, CD22, CD79b, Nectin-4, HER2, EGFR, EGFRvIII, cMET, FGFR-2, FGFR-3, AXL, HER3, CD166, CEACAM5, GPNMB, mesothelin, LIV1A, tissue factor (TF), CD71, CD228, FRα, NaPi2b, Trop-2, PSMA, CD70, STEAP1, P Cadherin, SLITRK6, LAMP1, CA9, GPR20 and CLDN18.2.

17. The antibody conjugate of any one of claims 1-8, wherein the antibody binds to a cell surface antigen in a tumour microenvironment.

18. The antibody conjugate of claim 17, wherein the cell surface antigen is one of Leucine-rich repeat containing 15 (LRRC15), FAPα, ANTXR1, TM4SF1, CD25, CD205, B7-H3 and HLA-DR.

19. The antibody conjugate of any one of claims 15-18, wherein the gene editing payload is capable of knocking out or disrupting a gene essential for cancer cell survival or replication.

20. The antibody conjugate of any one of claims 1-8, wherein the antibody binds to a T cell surface antigen.

21. The antibody conjugate of claim 20, wherein the T cell surface antigen is one of CD4, CD8, CD3, CTLA-4, TCR, TCRα and TCRβ.

22. The antibody conjugate of claim 20 or claim 21, wherein:
(a) the gene editing payload is capable of knocking out or disrupting PD-1, or
(b) the gene editing payload is capable of knocking out or disrupting a gene essential for T cell survival, optionally wherein the T cell surface antigen is CTLA-4.

23. The antibody conjugate of any one of claims 20-22 for use in treating cancer.

24. The antibody conjugate of any one of claims 1-8, wherein the antibody binds to a dendritic cell surface antigen.

25. The antibody conjugate of claim 24, wherein the dendritic cell surface antigen is one of HLA-DR, CD40, CD1c, Dectin 1, Dectin 2, CD141, CLEC9A, XCR1, CD303, CD304, CD123, CD14, CD209, Factor XIIIA, CD16, CX3CR1 and SLAN.

26. The antibody conjugate of claim 25, wherein the dendritic cell surface antigen is HLA-DR.

27. The antibody conjugate of claim 26, wherein the dendritic cell surface antigen is CD40.

28. The antibody conjugate of any one of claims 24-27, wherein the gene editing payload is capable of knocking out or disrupting CD40.

29. The antibody conjugate of any one of claims 24-28, for use in preventing transplant rejection.

30. An ex vivo method for gene editing comprising administering the antibody conjugate of any one of claims 1-8 to a population of cells comprising cells that express a surface antigen which is specifically bound by the antibody.

31. The method of claim 30, wherein the cells that express the surface antigen are:
(a) HSC, and wherein the antibody conjugate is as defined in one of claims 9-14,
(b) cancer cells, and wherein the antibody conjugate is as defined in any one of claims 15, 16 and 19,
(c) cells of the tumour microenvironment, and wherein the antibody conjugate is as defined in any one of claims 17-19,
(d) T cells, and wherein the antibody conjugate is as defined in any one of claims 20-22, or
(e) dendritic cells, and wherein the antibody conjugate is as defined in any one of claims 24-28.

32. An antibody conjugate comprising an anti-CD34 antibody and a nanoparticle conjugated to the antibody, wherein the nanoparticle comprises a payload.

33. An antibody conjugate comprising an anti-gpr56 antibody and a nanoparticle conjugated to the antibody, wherein the nanoparticle comprises a payload.

34. The antibody conjugate of claim 32 or claim 33, wherein the payload is a therapeutic payload.

35. The antibody conjugate of claim 34, wherein the therapeutic payload is a medicament for cancer therapy, such as a chemotherapy drug.

36. The antibody conjugate of claim 35, wherein the therapeutic payload is a toxin, such an alkylating agent.

37. An anti-CD34 antibody, comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),
wherein the VH comprises an HCDR1 comprising the amino acid sequence of SEQ ID NO: 1; an HCDR2 comprising the amino acid sequence of SEQ ID NO: 2; and an HCDR3 comprising the amino acid sequence of SEQ ID NO: 3; and the VL comprises an LCDR1 comprising the amino acid sequence of SEQ ID NO: 4; an LCDR2 comprising the amino acid sequence of SEQ ID NO: 5; and an LCDR3 comprising the amino acid sequence of SEQ ID NO: 6.

38. An anti-Gpr56 antibody, comprising a heavy chain variable region (VH) comprising three heavy chain complementarity determining regions (HCDR1, HCDR2, and HCDR3) and a light chain variable region (VL) comprising three light chain complementarity determining regions (LCDR1, LCDR2, and LCDR3),
wherein the VH comprises an HCDR1 comprising the amino acid sequence of SEQ ID NO: 7; an HCDR2 comprising the amino acid sequence of SEQ ID NO: 8; and an HCDR3 comprising the amino acid sequence of SEQ ID NO: 9; and the VL comprises an LCDR1 comprising the amino acid sequence of SEQ ID NO: 10; an LCDR2 comprising the amino acid sequence of SEQ ID NO: 11; and an LCDR3 comprising the amino acid sequence of SEQ ID NO: 12.

39. A method of making an antibody conjugate, comprising:
modifying an antibody heavy chain coding sequence to introduce a cysteine residue at or near the C-terminal end of the heavy chain constant region;
producing a modified antibody from the modified sequence, wherein the modified antibody comprises a cysteine residue at or near the C-terminal end of the heavy chain constant region, wherein said cysteine residue has a free thiol group that is not covalently bonded to another cysteine residue;
obtaining a nanoparticle comprising one or more polyethylene glycol (PEG) groups; and
conjugating the nanoparticle to the antibody via a site-specific maleimide linkage, wherein the cysteine residue at or near the C-terminal end of the heavy chain constant region is covalently bonded to one of the one or more PEG groups of the nanoparticle.

40. A method of treating or ameliorating the symptoms of a genetic disorder comprising administering to a patient a composition comprising an antibody conjugate, wherein said antibody conjugate comprises an antibody and a nanoparticle conjugated to the antibody, and said nanoparticle comprises a gene editing payload.

41. An antibody conjugate comprising an antibody and a nanoparticle conjugated to the antibody for use in the treatment or amelioration of the symptoms of a genetic disorder, wherein the nanoparticle comprises a gene editing payload.

42. The method of claim 40, or the antibody conjugate for use of claim 41, wherein the antibody conjugate is as defined in any one of claims 1-28 and 34-36.

43. A method of treating or ameliorating the symptoms of a disease comprising administering to a patient a composition comprising an antibody conjugate, wherein said antibody conjugate comprises an anti-gpr56 antibody and a nanoparticle conjugated to the antibody, and said nanoparticle comprises a therapeutic payload.

44. An antibody conjugate comprising an anti-gpr56 antibody and a nanoparticle conjugated to the antibody for use in the treatment or amelioration of the symptoms of a disease, wherein the nanoparticle comprises a therapeutic payload.

45. The method of claim 43, or the antibody conjugate for use of claim 44, wherein the antibody is as defined in claim 38.

46. A method of treating or ameliorating the symptoms of a disease comprising administering to a patient a composition comprising an antibody conjugate, wherein said antibody conjugate comprises an anti-CD34 antibody and a nanoparticle conjugated to the antibody, and said nanoparticle comprises a therapeutic payload.

47. An antibody conjugate comprising an anti-CD34 antibody and a nanoparticle conjugated to the antibody for use in the treatment or amelioration of the symptoms of a disease, wherein the nanoparticle comprises a therapeutic payload.

48. The method of claim 46, or the antibody conjugate for use of claim 47, wherein the antibody is as defined in claim 37.


SEQUENCE LISTING (94 sequences)

SEQ ID NO: 1 - protein, 8 aa, Homo sapiens
GFTFSRYG

SEQ ID NO: 2 - protein, 8 aa, Homo sapiens
IWYDGRNK

SEQ ID NO: 3 - protein, 10 aa, Homo sapiens
ASPIVGGTNY

SEQ ID NO: 4 - protein, 6 aa, Homo sapiens
QSVSSD

SEQ ID NO: 5 - skipped

SEQ ID NO: 6 - protein, 9 aa, Homo sapiens
QQYNNWPIT

SEQ ID NO: 7 - protein, 117 aa, Homo sapiens
QGQLVESGGGVVQPGRSLRLSCAASGFTFSRYGMHWVRQAPGKGLEWVAVIWYDGRNKEYGDSVKGRFTISRDNSKNTLYLQMNSLRAEDTAVYYCASPIVGGTNYWGQGTLVTVSS

SEQ ID NO: 8 - protein, 107 aa, Homo sapiens
EIVMTQSPATLSVSPGERATLSCRASQSVSSDLAWYQQKPGQAPRLLIYGASTRATGIPARFSGSGSGTELTLTISSLQSEDFAVYYCQQYNNWPITFGQGTRLEIK

SEQ ID NO: 9 - protein, 8 aa, Homo sapiens
GFTFSTYG

SEQ ID NO: 10 - protein, 8 aa, Homo sapiens
IWYDGSNK

SEQ ID NO: 11 - protein, 9 aa, Homo sapiens
ARHLDSFDI

SEQ ID NO: 12 - protein, 6 aa, Homo sapiens
QSISSY

SEQ ID NO: 13 - skipped

SEQ ID NO: 14 - protein, 9 aa, Homo sapiens
QQRDYWPIT

SEQ ID NO: 15 - protein, 116 aa, Homo sapiens
QVQLVESGGGVVQPGRSLRLSCTASGFTFSTYGMHWVRQAPGKGLEWVAVIWYDGSNKYYADSVKGRFTISRDNSKNTLYLQMNSLRAEDTAVYYCARHLDSFDIWGQGTMVTVSS

SEQ ID NO: 16 - protein, 107 aa, Homo sapiens
EIVLAQSPATLSLSPGERATLSCRASQSISSYLAWYQQKPGQAPRLLIYDAFNRATGIPARFSGSGSGTAFTLTISSLEPEDFAVYYCQQRDYWPITFGQGTRLEIK

SEQ ID NO: 17 - DNA, 20 bp, Artificial Sequence
  Synthetic
cttgtcaaggctattggtca

SEQ ID NO: 18 - protein, 17 aa, Artificial Sequence
  Synthetic
LEQNKTSSCAEFKKDRG

SEQ ID NO: 19 - DNA, 20 bp, Artificial Sequence
  Synthetic
cattgccctcaacgaccact

SEQ ID NO: 20 - DNA, 20 bp, Artificial Sequence
  Synthetic
ggtggtccaggggtcttact

SEQ ID NO: 21 - DNA, 19 bp, Artificial Sequence
  Synthetic
gccctggcccacaagtatc

SEQ ID NO: 22 - DNA, 23 bp, Artificial Sequence
  Synthetic
gcccttcataatatcccccagtt

SEQ ID NO: 23 - DNA, 20 bp, Artificial Sequence
  Synthetic
ggtgaccgttttggcaatcc

SEQ ID NO: 24 - DNA, 20 bp, Artificial Sequence
  Synthetic
gtatctggaggacagggcac

SEQ ID NO: 25 - DNA, 20 bp, Artificial Sequence
  Synthetic
atcttcttcaaggacgacgg

SEQ ID NO: 26 - DNA, 20 bp, Artificial Sequence
  Synthetic
ggctgttgtagttgtactcc

SEQ ID NO: 27 - DNA, 20 bp, Artificial Sequence
  Synthetic
acggctgacaaaagaagtcc

SEQ ID NO: 28 - DNA, 18 bp, Artificial Sequence
  Synthetic
gggtttctcctccagcat

SEQ ID NO: 29 - RNA, 20 nt, Artificial Sequence
  Synthetic
gggcgaggagcuguucaccg

SEQ ID NO: 30 - DNA, 20 bp, Artificial Sequence
  Synthetic
cttgtcaaggctattggtca

SEQ ID NO: 31 - RNA, 20 nt, Artificial Sequence
  Synthetic
cccgcuccucgacaaguggc

SEQ ID NO: 32 - protein, 102 aa, Artificial Sequence
  Synthetic
GVQCQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAR

SEQ ID NO: 33 - protein, 129 aa, Artificial Sequence
  Synthetic
GVQCQGQLVESGGGVVQPGRSLRLSCAASGFTFSFYGMHWVRQAPGKGLEWVAVINYDGRNKEYGDSVKGRFTISRDNSKNTLYLQMNSLRAEDTAVYYCASPIVGGTNYWGQGTLVTVSSAETTAPSV

SEQ ID NO: 34 - protein, 129 aa, Artificial Sequence
  Synthetic
GVQCQGQLVESGGGVVQPGRSLRLSCAASGFTFSRYGMHWVRQAPGKGLEWVAVIWYDGRNKEYGDSVKGRFTISRDNSKNTLYLQMNSLRAEDTAVYYCASPIVGGTNYWGQGTLVTVSSAETTAPSV

SEQ ID NO: 35 - protein, 134 aa, Artificial Sequence
  Synthetic
GVQCQVQLVESGGDVVQPGRSLRLSCEASGFTFRNYGMHWVRQAPGKGLEWVAAIWYDGSKKFYSDSVKGRFTISRDNSKNTLYLQMNSLRAEDTAVYYCARYFVSGSYFSPFDYWGQGTLVTVSSAQTTAPSV

SEQ ID NO: 36 - protein, 132 aa, Artificial Sequence
  Synthetic
SCQCQVQLVESGGGVVQPGRSLRLSCAASGFTFSSYGMHWVRQAPGKGLEWVAVIWYDGSKKYYTDSVKGRFTISRDNSKNTLYLQMNSLRAEDTAVFYCARWGSGNFDSFDIWGQGTMVTVSSARTTAPSV

SEQ ID NO: 37 - protein, 132 aa, Artificial Sequence
  Synthetic
GVQCQVQLVESGGGVVQPGRSLRLSCAASGFTFSNYGMHWVRQAPDKGLEWVAVIWYDGSKKYYADSVKGRFTISRDDSKNTLYLQMNSLRAEDTAVYYCARWGELRYDAFDIWGQGTMVTVSSARTTAPSV

SEQ ID NO: 38 - protein, 98 aa, Artificial Sequence
  Synthetic
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 39 - protein, 126 aa, Artificial Sequence
  Synthetic
QVQLQESGPGLVKPSGTLSLTCAVSGGSISSRNWWTWVRQPPGKGLEWIGEIFHSDNTNYNPSLKSRVTISVDKSKNQFSLMLTSVTAADTAVYYCAREGYSYGLDYWGQGTLVTVSSAQTTAPSV

SEQ ID NO: 40 - protein, 126 aa, Artificial Sequence
  Synthetic
QVQLQESGPGLVKPSGTLSLTCAVSGGSISSRNWWTWVRQPPGKGLEWIGDIFHSDNTNYNPSLKSRVTISIDKSRNQFFLKLTSVTAADTAVYYCVREGYSYGLDYWGQGTLVTVSSAQTTAPSV

SEQ ID NO: 41 - protein, 126 aa, Artificial Sequence
  Synthetic
QVQLQESGPGLVKPSGTLSLTCAVSGGSISSRNWWTWVRQPPGKGLEWIGDIFHSDNTNYNPSLKSRVTISVDKSRNQFSLRLTSVTAADTAVYYCVREGYSYGLDYWGQGTLVTVSSAQTTAPSV

SEQ ID NO: 42 - protein, 126 aa, Artificial Sequence
  Synthetic
QVQLQESGPGLVKPSGTLSLTCAVSGGSISSRNWWTWVRQPPGKGLEWIGDIFHSENTNYNPSLKSRLTISVDKSKNQFSLKLTSVTAADTAVYYCVREGYGYGLDYWGQGTLVTVSSAQTTAPSV

SEQ ID NO: 43 - protein, 126 aa, Artificial Sequence
  Synthetic
QVKLQESGPGLVKPSGTLSLTCAVSGGSISSRNWWTWVRQPPGKGLEWIGDIFHSDNTNYNPSLKSRLTISVDKSKNQFSLKLTSVTAADTAVYYCVREGYSYGLDYWGQGTLVTVSSAQTTAPSV

SEQ ID NO: 44 - protein, 98 aa, Artificial Sequence
  Synthetic
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 45 - protein, 130 aa, Artificial Sequence
  Synthetic
EMQLLESGGGLVQPGGSLRLSCAASGFTFGHFAMSWVRQVPGKGLDWVSAISGGGSFTYSADSVKGRFTTSRDNSKNTLYLQMSSLRAEDTALYYCAKDQGYDYSWYYFDFWGQGTLVTVSSAQTTAPSV

SEQ ID NO: 46 - protein, 130 aa, Artificial Sequence
  Synthetic
EMQLLESGGGLVQPGGSLRLSCAASGFTFGHYAMTWVRQVPGKGLDWVSAISGGGTFTHYADSVKGRFTTSRDNSKNTLYLQMSSLRAEDTALYYCAKDQGYDYSWYYFDYWGQGTLVTVSSAQTTAPSV

SEQ ID NO: 47 - protein, 130 aa, Artificial Sequence; identical to SEQ ID NO: 46
  Synthetic

SEQ ID NO: 48 - protein, 95 aa, Artificial Sequence
  Synthetic
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 49 - protein, 127 aa, Artificial Sequence
  Synthetic
DIQMTQSPSTLSASVGDRVTITCRASQSISSWLAWYQQRPGKAPKLLIYKASSLESGVPSRFSGRGSGTEFTLTISSLQPDDFATYYCQQYKSHSITFGQGTRLEIKRADAAPTVSIFPPSTEQLAT

SEQ ID NO: 50 - protein, 127 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPDDFATYYCQQYKSHSITFGQGTRLEIKRADAAPTVSIFPPSTEQLAT

SEQ ID NO: 51 - protein, 127 aa, Artificial Sequence; identical to SEQ ID NO: 50
  Synthetic

SEQ ID NO: 52 - protein, 128 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYSMYTFGQGTKLEIKRADAAPTVSIFPPSTEQLAT

SEQ ID NO: 53 - protein, 126 aa, Artificial Sequence
  Synthetic
DIQMTQSPSTLSASVGDRVTITCRASQSISSWLAWYQQKPGKAPKLLISKASSLESGVPSRFSGSGSGTEFTLTISSLQPDDFATYYCQQYKNYWTFGQGTKVEIKRADAAPTVSIFPPSTEQLAT

SEQ ID NO: 54 - protein, 100 aa, Artificial Sequence
  Synthetic
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 55 - protein, 131 aa, Artificial Sequence
  Synthetic
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLTFGGGTKVEIKRADAAPTVSIFPPSTEQLAT

SEQ ID NO: 56 - protein, 131 aa, Artificial Sequence; identical to SEQ ID NO: 55
  Synthetic

SEQ ID NO: 57 - protein, 131 aa, Artificial Sequence; identical to SEQ ID NO: 55
  Synthetic

SEQ ID NO: 58 - protein, 131 aa, Artificial Sequence; identical to SEQ ID NO: 55
  Synthetic

SEQ ID NO: 59 - protein, 131 aa, Artificial Sequence; identical to SEQ ID NO: 55
  Synthetic

SEQ ID NO: 60 - protein, 95 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 61 - protein, 127 aa, Artificial Sequence
  Synthetic
EIVMTQSPATLSVSPGERATLSCRASQSVSSDLAWYQQKPGQAPRLLIYGASTRATGIPARFSGSGSGTELTLTISSLQSEDFAVYYCQQYNNWPITFGQGTRLEIKRADAAPTVSIFPPSTEQLAT

SEQ ID NO: 62 - protein, 127 aa, Artificial Sequence; identical to SEQ ID NO: 61
  Synthetic

SEQ ID NO: 63 - protein, 95 aa, Artificial Sequence
  Synthetic
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 64 - protein, 127 aa, Artificial Sequence
  Synthetic
DIQLTQSPSFLSASVGDRVTITCRASQGISSYLAWYQHRPGKAPKLLIYSASTLFSGVPSRFSGSGSGTDFTLTITSLQPADFATYYCQQLDSYPLTFGGGTKVEIKRADAAPTVSIFPPSTEQLAT

SEQ ID NO: 65 - protein, 127 aa, Artificial Sequence
  Synthetic
DIQLTQSPSFLSASVGDRVTITCRASQGISRYLAWYQYKPGKAPKLLIYSASTLLGGVPSRFSGSGSGTEFTLTISSLQPEDFATYYCQHLNSYPLTFGGGTKVEIKRADAAPTVSIFPPSTEQLAT

SEQ ID NO: 66 - protein, 100 aa, Artificial Sequence
  Synthetic
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 67 - protein, 131 aa, Artificial Sequence
  Synthetic
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWLTFGQGTRLEIKRADAAPTVSIFPPSTEQLAT

SEQ ID NO: 68 - protein, 98 aa, Artificial Sequence
  Synthetic
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 69 - protein, 121 aa, Artificial Sequence
  Synthetic
QVQLVESGGGVVQPERSLRLSCAASGIIFSSYGMHWVRQAPGKGLEWVAVIWYDGSNKYYADSVKGRFTISRDNSKNTLYLQMNSLRAEDTAVYYCAGNWGKDAFDIWGQGTMVTVSSAQT

SEQ ID NO: 70 - protein, 121 aa, Artificial Sequence
  Synthetic
QVQLVESGGGVVQPGRSLRLSCVASGFTLSLNGMHWVRQAPGKGLEWVAVIWYDGSNRYYVDSVKGRFTISRDNSKNTLYLQMNSLRAEDTAVYYCARESFLGTFDYWGQGTLVTVSSAQT

SEQ ID NO: 71 - protein, 122 aa, Artificial Sequence
  Synthetic
QVQLVESGGGVVQPGRSLRLSCAASGFIFSTYGIHWVRQVPGKGLEWVAVIWYDGSNKYYADSVKGRFTLSRDNSKNMVYLQMNSLRAEDTAVYYCVREGGWNYVMDVWGQGTSVTVSSAQT

SEQ ID NO: 72 - protein, 126 aa, Artificial Sequence
  Synthetic
QVQLVESGGGVAQPGRSLRLSCAASGFTFSSSNMHWVRQAPGKGLEWVAFIWYDGSNKYYADSVKGRFTISRDNSKNTLYLQMNSLRAEDTAVYYCAGEGDTSGWFPYYFDYWGQGTLVTVSSAQT

SEQ ID NO: 73 - protein, 119 aa, Artificial Sequence
  Synthetic
QVQLVESGGGVVQPGRSLRLSCTASGFTFSTYGMHWVRQAPGKGLEWVAVIWYDGSNKYYADSVKGRFTISRDNSKNTLYLQMNSLRAEDTAVYYCARHLDSFDIWGQGTMVTVSSAQT

SEQ ID NO: 74 - protein, 124 aa, Artificial Sequence
  Synthetic
QVQLVESGGGVVQPGRSLRLSCAASGFTFSRYGMHWVRQAPGKGLEWVAFIWHDGSFEYSADSVKGRFTISRDNSKNTLYLHMNSLRAGDTAVYYCARGIAAAGSPHFDYWGQGTLVTVSSART

SEQ ID NO: 75 - protein, 122 aa, Artificial Sequence
  Synthetic
QVQVVESGGGVVQPGRSLRLSCAASGFTFSSYGMHWVRQAPGKGLEWVAVIWYDGSNEYYADSVKGRFTISRDNSKNTLFLEMNSLRAEDTAMYYCASESPLTAVVDYWGQGTLVTVSSART

SEQ ID NO: 76 - protein, 98 aa, Artificial Sequence; identical to SEQ ID NO: 38
  Synthetic

SEQ ID NO: 77 - protein, 130 aa, Artificial Sequence
  Synthetic
QVQLQESGPGLVKPSGTLSLTCAVSGGSISSRNWWSWVRQPPGKGLEWIGEIYHSGNNDYNPSLKSRVTISVDKSKNQFSLKLSSVTAADTAVYFCARGGYYFVSGNYYKPDAFDIWGQGTMVTVSSAQT

SEQ ID NO: 78 - protein, 95 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 79 - protein, 113 aa, Artificial Sequence
  Synthetic
EIVLAQSPATLSLSPGERATLSCRASQSISSYLAWYQQKPGQAPRLLIYDAFNRATGIPARFSGSGSGTAFTLTISSLEPEDFAVYYCQQRDYWPITFGQGTRLEIKRADAAP

SEQ ID NO: 80 - protein, 95 aa, Artificial Sequence; identical to SEQ ID NO: 60
  Synthetic

SEQ ID NO: 81 - protein, 114 aa, Artificial Sequence
  Synthetic
EIVMTQSPATLSVSPGERVTLSCRASQSVSSNLAWYQQKPGQAPRLLIYGASTRATGIPARFSGSGSGTEFTLTISSLQSEDFAVYYCQQYNNWPPFTFGPGTKVDIKRADAAP

SEQ ID NO: 82 - protein, 114 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNIWPPYTFGQGTKLEIKRADAAP

SEQ ID NO: 83 - protein, 114 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNYWPPFTFGPGTTVDIKRADAAP

SEQ ID NO: 84 - protein, 95 aa, Artificial Sequence; identical to SEQ ID NO: 63
  Synthetic

SEQ ID NO: 85 - protein, 113 aa, Artificial Sequence
  Synthetic
EIQLTQSPSFLSASVGGRVTITCRASQGISSYLAWYQHKPGKAPKLLIYAASTLQSGVPSRFSGSGSGTEFTLTVNSLQPEDFATYYCHQLDSYPFTFGGGTKVEIQRADAAP

SEQ ID NO: 86 - protein, 113 aa, Artificial Sequence
  Synthetic
DIQLTQSPSFLSASVGDRVTITCRASQGISSYLAWYQQKPGKAPKLLIYAASTLQSGVPSRFSGSGSGTEFTLTISSLQPEDFATYYCQQLNSYPYTFGQGTKLEIKRADAAP

SEQ ID NO: 87 - protein, 113 aa, Artificial Sequence; identical to SEQ ID NO: 86
  Synthetic

SEQ ID NO: 88 - protein, 113 aa, Artificial Sequence
  Synthetic
DIQLTQSPSFLSASVRDRVTITCRASQGISSYLAWYQQKPGKAPKLLIYAASTLQSGVPSRFSGSGSGTEFTLTISSLQPEDYATYYCQQLYDFPLTFGGGTKVAIKRADAAP

SEQ ID NO: 89 - protein, 95 aa, Artificial Sequence; identical to SEQ ID NO: 48
  Synthetic

SEQ ID NO: 90 - protein, 114 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPADFASYYCQHYSSYSPYTFGQGTKLEIKRADAAP

SEQ ID NO: 91 - protein, 113 aa, Artificial Sequence
  Synthetic
QAVVTQESALTTSPGETVTLTCRSSTGAVTTSNYANWVQEKPDHLFTGLIGVTNNRAPGVPARFSGSLIGDKAALTITGAQTEDEAIYFCALWYSNHWVFGGGTKLTVLGQPK

SEQ ID NO: 92 - DNA, 30 bp, Artificial Sequence
  Synthetic
gccttgaccaatagccttgacaaggcaaac

SEQ ID NO: 93 - DNA, 30 bp, Artificial Sequence; identical to SEQ ID NO: 92
  Synthetic

SEQ ID NO: 94 - DNA, 30 bp, Artificial Sequence; identical to SEQ ID NO: 92
  Synthetic